(19) United States
(12) Patent Application Publication
(10) Pub. No.: US 2023/0010299 A1
(43) Pub. Date: Jan. 12, 2023

(54) HETEROCYCLIC TRPML1 AGONISTS

(71) Applicant: LIBRA THERAPEUTICS, INC., San Diego, CA (US)

(72) Inventors: Paolo PEVARELLO, Bresso (MI) (IT); Valentina CUSANO, Bresso (MI) (IT); Mariangela SODANO, Bresso (MI) (IT); Domenica TORINO, Bresso (MI) (IT); Rocco VITALONE, Bresso (MI) (IT); Chiara LIBERATI, Bresso (MI) (IT); Francesco PISCITELLI, Bresso (MI) (IT)

(21) Appl. No.: 17/776,050

(22) PCT Filed: Nov. 12, 2020
(86) PCT No.: PCT/IB2020/060659

§ 371 Date: May 11, 2022

(30) Foreign Application Priority Data
Nov. 13, 2019 (EP) 19208888.8

(51) Int. Cl. C07D 409/12; C07D 295/135; C07C 311/21; C07D 471/08; C07D 295/205; C07D 209/08; C07D 217/04; C07D 401/12; C07D 409/14; C07D 211/14; C07D 211/62; C07D 223/08; C07D 267/10; C07D 307/79; C07D 215/36; C07D 417/14; C07D 265/30

(52) U.S. Cl. CPC C07D 409/12; C07D 295/135; C07C 311/21; C07D 471/08; C07D 295/205; C07D 209/08; C07D 217/04; C07D 401/12; C07D 409/14; C07D 211/14; C07D 211/62; C07D 223/08; C07D 267/10; C07D 307/79; C07D 215/36; C07D 417/14; C07D 265/30

(57) ABSTRACT

Disclosed is a compound of formula (I)

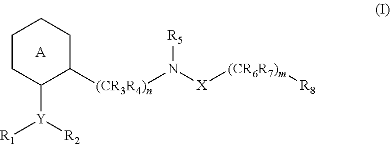

or a stereoisomer thereof, or a salt of any of the foregoing and to processes for its preparation. The compounds of formula (I) are useful in the treatment TRPML1-mediated disorders or diseases.

### CROSS-REFERENCE TO RELATED APPLICATIONS

This application is the U.S. national phase of International Application No. PCT/IB2020/060659 filed Nov. 12, 2020 which designated the U.S. and claims priority to EP 19208888.8 filed Nov. 13, 2019, the entire contents of each of which are hereby incorporated by reference.

### BACKGROUND OF THE INVENTION

### Field of the Invention

Disclosed herein are novel heterocyclic compounds having Transient Receptor Potential Cation Channel, Mucolipin subfamily, member 1 (TRPML1) agonistic properties, pharmaceutical compositions comprising these compounds, chemical processes for preparing these compounds and their use in the treatment or prophylaxis of diseases associated with TRPML1 receptor activity in animals, in particular humans.

### Description of the Related Art

TRPML1, also named Mucolipin-1, is a ligand-gated cation channel expressed mostly in intracellular organelles like the late endosome and lysosome of many mammalian cells. This channel is member of the large family of Transient receptor potential (TRP) channels and has, with TRPML2 and TRPML3, two close analogues. Loss-of-function mutations in the gene encoding for TRPML1, the 12′000 base pair gene MCOLN-1 located in human chromosome 19p13, are the direct cause of Type IV mucolipidosis (MLIV), an autosomal recessive lysosomal storage disease.

At the molecular level, TRPML1 is a Ca²⁺-permeable, non-selective cation channel formed of four six-transmembrane spanning proteins each of 580 amino acids. The channel opens upon binding of its endogenous ligand phosphatidylinositol-3,5-bisphosphate (PtdIns(3,5)P2)) to its pore region. Channel activity is modulated by pH and PtdIns(4,5)P2 levels. TRPML1 is an inwardly rectifying channel permeable for different mono- and divalent cations, including Na⁺, K⁺, Ca²⁺ and Fe²⁺. Its N-terminal AP1 sequence targets the channel to the lysosome while a C-terminal AP2 sequence is responsible for intracellular trafficking and internalization. In addition, TRPML1 has four putative N-linked glycosylation sites in its luminal loop between TM1 and 2. It is reported that TRPML channels can be formed as homo-tetramers (e.g. TRPML1, TRPML2, TRPML3) but also in some cases as hetero-tetramers where one channel is composed of different members of the TRPML family.

TRPML1 is found in all mammalian tissues with highest expression levels in brain, spleen, liver, kidney and heart. Expression is found in many cell types, including neurons, myeloid cells, macrophages, microglia, podocytes and muscle cells. TRPML1 is involved in function of late endosome/lysosomes (LELs), more specifically in protein trafficking and lysis as well as autophagy.

Lysosomes are organelles filled with hydrolytic enzymes, characterized by low luminal pH of about 5, a high luminal Ca²⁺ concentration of about 0.5 mM and a membrane polarization of about +60 mV.

TRPML1 in LELs is reported to be responsible for the formation of transport vesicles and required for the reformation of lysosomes from LEL hybrid organelles and autolysosomes, mostly due to its Ca²⁺ permeability. It seems also important for iron release from the lysosome after degradation of iron-binding proteins like cytochrome C. In addition, TRPML1 is reported to regulate autophagy, probably in an mTOR-independent manner, by promoting TFEB translocation to the nucleus via calcineurin activation.

In MLIV, the lack of functional TRPML1 leads to severe intellectual disability, motor deficits, retinal degeneration and systemic symptoms leading to a strongly reduced life expectancy. Cells from MLIV patients show increased autophagosomes, accumulation of lysofuscin and lipid accumulation in the lysosomes.

Failure of TRPML1-dependent autophagosome-lysosome fusion is also thought to impair clearance of apoptotic neurons by macrophages and microglia cells. Experimental results suggest involvement of TRPML1 in neurodegenerative diseases like Alzheimer's and amyotrophic lateral sclerosis (ALS). For example, Alzheimer's disease related loss-of-function mutations in presenilin 1 lead to dysregulation of lysosomal Ca²⁺ homeostasis via TRPML1 modulation. On the other side, over-expression of TRPML1 in rodent Alzheimer's models reduced neuronal apoptosis and rescued memory impairments. Pharmacological activation of TRPML1 showed similar effects, clearing accumulated sphingolipids and Aβ peptides from lysosomes. In another study TRPML1 activation was sufficient to upregulate lysosomal exocytosis, rescue defective α-syn secretion and prevent α-syn accumulation in iPSC-derived dopaminergic neurons from patients expressing mutant PARK9. Similarly, TRPML1 activation rescued motor neurons from death and ER stress induced by the cycad neurotoxin beta-methylamino-L-alanine, L-BMAA as a model for ALS.

### SUMMARY OF THE INVENTION

Therefore, it is desired to develop TRPML1 modulators to rescue impaired lysosomal function and cellular autophagy in neurodegenerative diseases.

WO2018005713 describes TRPML1 agonist molecules. Unfortunately, despite widespread interest for several years across the pharmaceutical industry, there are no drug-like small molecule agonists or positive allosteric modulators of any kind of TRPML1.

Consequently, there is still an unmet need for compounds which can efficiently stimulate TRPML1 and that can be delivered to the different target organs which are sites of any TRPML1-mediated pathology. Such compounds are provided herein.

Various embodiments of the compounds provided herein are presented hereafter.

### DESCRIPTION OF THE PREFERRED EMBODIMENTS

Disclosed herein are compounds of the following formula (I)

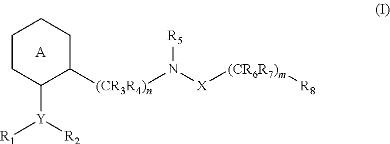

or a stereoisomer thereof, or a salt of any of the foregoing, wherein:

A is a six membered aromatic, heteroaromatic or aliphatic ring, optionally substituted by one or more substituents selected from the group consisting of C₁-C₄ alkyl, C₁-C₄ alkyloxy, C₃-C₆ cycloalkyl, halogen, and cyano.

Y is chosen from CH, or N;

each of R₁ and R₂ independently is a C₁-C₄ alkyl group or R₁ and R₂ taken together with Y form a cyclic compound being a four, five, six or seven membered heteroaliphatic compound, containing at least one nitrogen atom, optionally fused with a six membered aromatic ring, optionally substituted by one or more substituents selected from the group consisting of:

halogen;

C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;

phenyl, benzyl, phenoxy, benzyloxy, pyrazin-2-yl-oxy and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;

a C₁-C₂ alkylene bridge;

R₁₁R₁₂N—CO— wherein each of R₁₁ and R₁₂ is C₁-C₄ alkyl;

X is —CO— or —S02-;

each of R₃, R₄, R₆ and R₇ independently is hydrogen or C₁-C₄ alkyl;

R₅ is independently hydrogen or C₁-C₄ alkyl;

each of m and n independently is 0 or 1;

R₈ is a five or six membered monocyclic or a nine or ten membered bicyclic aromatic compound, optionally substituted by one or more substituents selected from the group consisting of halogen, bromo, or pentafluorosulfanyl (SF₅), or trifluoromethylthio (SCF₃), C₁-C₄ alkyl or C₁-C₆ cycloalkyl optionally substituted by a cyano group, —NO₂, C₁-C₄ alkyloxy, C₁-C₄ alkylthio, —SO₂NR₉R₁₀ or —NR₉R₁₀, wherein each of R₉ and R₁₀ independently is hydrogen or C₁-C₄ alkyl or R₉ and R₁₀ are taken together to form a five or six membered heteroaliphatic ring, optionally containing one or two oxygen atoms;

provided that:
when X is —CO— then R₅ is C1-C4 alkyl
when X is —SO₂— then R₅ is hydrogen;
when A is phenyl, X is —SO₂, m and n are both 0 and R₁ and R₂ taken together form a piperazine substituted by phenyl optionally substituted as defined above, then R₈ is not phenyl optionally substituted as defined above;
when A is phenyl, X is —SO₂, m and n are both 0 and R₁ and R₂ taken together form an unsubstituted piperidine, pyrrolidine, or azepane, then R₈ is not thiophene optionally substituted as defined above;
when A is cyclohexyl, X is —SO₂, m and n are both 0 and R₁ and R₂ taken together form an unsubstituted morpholine, then R₈ is not phenyl optionally substituted as defined above.

C₁-C₄ alkyl group means a straight or branched alkyl chain containing 1 to 4 carbon atoms such as methyl, ethyl, n-propyl, sec-propyl, n-butyl, sec-butyl and tert-butyl, preferably is methyl, ethyl or tert-butyl, most preferably methyl.

Preferably, A is phenyl or cyclohexyl.

Preferably Y is N.

Preferably Y is N and R₁ and R₂ are methyl or R₁ and R₂ taken together with Y form a piperidine, piperazine, pyrrolidine, azetidine, azepane, 2-azabicyclo[2.2.1]heptane, 1,4-oxazepane, morpholine, 1,2,3,4-tetrahydroisoquinoline, 2,3-dihydro(1H)indole, wherein each of said moieties is optionally substituted by: halogen, preferably fluorine or chlorine; C₁-C₄ alkyl, preferably methyl, ethyl, n-propyl or tert-butyl; C₁-C₄ alkyloxy, preferably methoxy; C₁-C₄ alkylcarbonyl (wherein the C₁-C₄ alkyl group is optionally substituted by one or more halogen, preferably fluorine or chlorine, or C₁-C₄-alkyloxy, preferably methoxy), preferably methylcarbonyl, methoxyethylcarbonyl; C₁-C₄ alkoxycarbonyl (wherein the C₁-C₄ alkyl group is optionally substituted by one or more halogen, preferably fluorine or chlorine or C₁-C₄-alkyloxy, preferably methoxy), preferably methoxycarbonyl, ethoxycarbonyl, tert-butoxycarbonyl, 2,2,2-trifluoroethoxycarbonyl; a compound containing an aromatic ring selected from phenyl, benzyl, phenoxy, benzyloxy, pyrazin-2-yl-oxy and pyridin-2-yl-oxy (wherein the aromatic ring is optionally substituted by one or more halogen, preferably fluorine or chlorine, or C₁-C₄-alkyloxy); and a C₁-C₂ alkylene bridge, preferably a methylene bridge.

Preferably, each of R₃, R₄, R₅, R₆ and R₇ are hydrogen or methyl, most preferably hydrogen.

Preferably, both m and n are 0 or one of m and n is 0 and the other is 1, most preferably they are both 0.

Preferably, R₈ is selected from the group consisting of phenyl, thiophene, benzothiophene, optionally substituted by one or more substituents selected from the group consisting of halogen, preferably fluorine or chlorine or bromo, or pentafluorosulfanyl (SF₅), or trifluoromethylthio (SCF₃); C₁-C₄ alkyl optionally substituted by a cyano group, preferably methyl, ethyl, n-propyl, tert-butyl or cyanomethyl; —NO₂; C₁-C₄ alkoxy, preferably methoxy, ethoxy; C₁-C₄ alkylthio, preferably methylthio, ethylthio; —SO₂NR₉R₁₀ or —NR₉R₁₀, wherein each of R₉ and R₁₀ independently is hydrogen or C₁-C₄ alkyl, preferably methyl, ethyl, most preferably both R₉ and R₁₀ are methyl, or R₉ and R₁₀ are taken together to form a five or six membered heteroaliphatic ring, optionally containing one or two oxygen atoms, preferably piperidine, pyrrolidine, morpholine, 1,3 dioxolane.

A preferred embodiment of the invention relates to compounds of formula (I) or a stereoisomer or a salt thereof as defined above wherein:

A is phenyl or 1,2 disubstituted cyclohexyl;

Y is N and R₁ and R₂ taken together form a cyclic compound selected from piperidine, pyrrolidine, azetidine, azepane, 2-azabicyclo[2.2.1]heptane, 4-phenylpiperidine, tert-butyl piperazine-1-carboxylate, 4-phenoxypiperidine, 4-(benzyloxy)piperidine, 4-methoxypiperidine, morpholine, dimethylamine, pyrrolidine, 4,4-difluoropiperidine, 3-phenylpiperidine, 3-methoxypiperidine, 1-(4-chloro-2-fluorophenyl)piperazine, 3-phenylpyrrolidine, 2,3-dihydro-1H-indole, 1,2,3,4-tetrahydroquinoline, methyl piperazine-1-carboxylate, 2,2,2-trifluoroethyl piperazine-1-carboxylate, ethyl piperazine-1-carboxylate, 1-(piperazin-1-yl)propan-1-one, 3-methoxy-1-(piperazin-1-yl)propan-1-one, 1-benzoylpiperazine, 2-(piperidin-4-yloxy)pyridine, 2-(piperidin-4-yloxy)pyrazine, tert-butyl piperidine-4-carboxylate, tert-butyl piperidine-1-carboxylate, 4-(pyridin-2-yl)piperidine, 4-(pyrazin-2-yl)piperidine, 4-(thiophene-2-carbonyl)piperazine, 4-(4-methyl-1,3-thiazole-5-carbonyl)piperazine, 4-(piperazin-1-yl)-2-(thiophen-2-yl)ethan-1-one, N-methyl-N-propylpiperazine-1-carboxamide, 4-(thiophene-2-carbonyl)piperazine, 4-(4-methyl-1,3-thiazole-5-carbonyl)piperazine, 4-(piperazin-1-yl)-2-(thiophen-2-yl)ethan-1-one, N-methyl-N-propylpiperazine-1-carboxamide, 3-(piperidin-4-yloxy)pyridine, azepan-1-yl, 4-methoxyazepan-4-yl, 1,4-oxazepan-4-yl, N-propylpiperidine-4-carboxamide, N-(2-methylpropyl)piperidine-4-carboxamide, 2-(piperidin-4-yloxy)pyrimidine, piperidin-4-yl 2,2-dimethylpropanoate.

both m and n are 0;

R₃, R₄, R₆ and R₇ are hydrogen;

X is —CO— or —SO₂—;

R₅ is hydrogen or methyl;

R₈ is selected from phenyl, thiophene or benzothiophene, optionally substituted by dimethylsulfamoyl, methyl, ethyl, propyl, tert-butyl, 1,3-dioxolan-2-yl, cyanomethyl, monoalkylamino, dialkylamino, piperidine-1-sulfonyl, pyrrolidine-1-sulfonyl, morpholine-4-sulfonyl, fluoro, chloro, bromo, SF₅, SCF₃, nitro, and C₁-C₄ alkoxy.

provided that:
when X is —CO— then R₅ is C₁-C₄ alkyl;
when X is —SO₂— then R₅ is hydrogen;
when A is phenyl, X is —SO₂, m and n are both 0 and R₁ and R₂ taken together form a piperazine substituted by phenyl optionally substituted as defined above, then R₈ is not phenyl optionally substituted as defined above;
when A is phenyl, X is —SO₂, m and n are both 0 and R₁ and R₂ taken together form an unsubstituted piperidine, pyrrolidine, or azepane, then R₈ is not thiophene optionally substituted as defined above;
when A is cyclohexyl, X is —SO₂, m and n are both 0 and R₁ and R₂ taken together form an unsubstituted morpholine, then R₈ is not phenyl optionally substituted as defined above.

A further preferred embodiment of the invention is a compound of formula (I) wherein:

A is phenyl;

Y is N;

R₁ and R₂ taken together with Y form a cyclic compound selected from piperidine, azepane, 1,4-oxazepane, pyrrolidine, morpholine, 2,3-dihydro-1H-indole, 1,2,3,4-tetrahydroisoquinoline optionally substituted by one or more substituents selected from the group consisting of:
halogen;
C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;
phenyl, benzyl, phenoxy, benzyloxy, pyrazin-2-yl-oxy, pyrimidin-2-yl-oxy, and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;
a C₁-C₂ alkylene bridge;
R₁₁R₁₂N—CO— wherein each of R₁₁ and R₁₂ is C₁-C₄ alkyl; or

R₁ and R₂ taken together with Y form a piperazine optionally substituted with —R₁₁R₁₂N—CO— wherein each of R₁₁ and R₁₂ is C₁-C₄ alkyl;

n and m and n are both 0;

R₃, R₄, R₆ and R₇ are hydrogen;

X is —SO₂— and R₅ is hydrogen; or

X is —CO— and R₅ is methyl;

R₈ is selected from phenyl, thiophene or benzothiophene, thiazole, furan, pyridine, isoxazole, indoline, dihydrobenzofuran, quinolone, optionally substituted by one or more groups selected from dimethylsulfamoyl, methyl, ethyl, propyl, tert-butyl, 1,3-dioxolan-2-yl, mono(C1-C6)alkylamino, di (C1-C6)alkylamino, methylsulfonyl, and C₁-C₄ alkoxy;

provided that:

when X is —SO₂, and R₁ and R₂ taken together form an unsubstituted piperidine, pyrrolidine, or azepane, then R₈ is not thiophene optionally substituted as defined above.

A further preferred embodiment of the invention is a compound of formula (I) wherein:

A is cyclohexyl;

Y is N;

R₁ and R₂ taken together with Y form a cyclic compound selected from piperidine, azepane, 1,4-oxazepane, piperazine, pyrrolidine, morpholine, 2,3-dihydro-1H-indole, 1,2,3,4-tetrahydroisoquinoline optionally substituted by one or more substituents selected from the group consisting of:
halogen;
C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;
phenyl, benzyl, phenoxy, benzyloxy, pyrazin-2-yl-oxy, pyrimidin-2-yl-oxy, and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;
C₁-C₂ alkylene bridge;
R₁₁R₁₂N—CO— wherein each of R₁₁ and R₁₂ is C₁-C₄ alkyl; or

n and m and n are both 0;

R₃, R₄, R₆ and R₇ are hydrogen;

X is —SO₂— and R₅ is hydrogen; or

X is —CO— and R₅ is methyl;

R₈ is selected from phenyl, thiophene or benzothiophene, thiazole, furan, pydine, isoxazole, indoline, dihydrobenzofuran, quinolone, substituted by one or more groups selected from dimethylsulfamoyl, methyl, ethyl, propyl, tert-butyl, 1,3-dioxolan-2-yl, mono(C1-C6)alkylamino, di (C1-C6)alkylamino, methylsulfonyl, and C₁-C₄ alkoxy;
provided that:
when X is —S₂, and R₁ and R₂ taken together formant unsubstituted morpholine, then R₈ is not phenyl optionally substituted as defined above.

In certain embodiments, a compound of formula (I) disclosed herein is chosen from the compounds set forth in Table 1 or a stereoisomer or a salt thereof.

TABLE 1

| Example | Chemical Name |
|---|---|
| 1 | N-[2-(piperidin-1-yl)phenyl]-1-benzothiophene-2-sulfonamide |
| 2 | 4-(dimethylamino)-N-[2-(piperidin-1-yl)phenyl]benzene-1-sulfonamide |
| 3 | N4-[2-(dimethylamino)phenyl]-N1,N1-dimethylbenzene-1,4-disulfonamide |
| 4 | N4-(2-{2-azabicyclo[2.2.1]heptan-2-yl}phenyl)-N1,N1-dimethylbenzene-1,4-disulfonamide |
| 5 | tert-butyl 4-(2-(4-(N,N-dimethylsulfamoyl)-N-methylbenzamido)phenyl)piperazine-1-carboxylate |
| 6 | N1,N1-dimethyl-N4-[2-(piperidin-1-yl)phenyl]benzene-1,4-disulfonamide |
| 7 | 2-[4-(dimethylsulfamoyl)phenyl]-N-[2-(pyrrolidin-1-yl)phenyl]acetamide |
| 8 | N4-[2-(2,3-dihydro-1H-indol-1-yl)phenyl]-N1,N1-dimethylbenzene-1,4-disulfonamide |
| 9 | N1,N1-dimethyl-N4-[2-(1,2,3,4-tetrahydroisoquinolin-2-yl)phenyl]benzene-1,4-disulfonamide |
| 10 | 4-(N,N-dimethylsulfamoyl)-N-methyl-N-(2-(4-(pyridin-2-yloxy)piperidin-1-yl)phenyl)benzamide |
| 11 | 3-(1,3-dioxolan-2-yl)-N-[2-(piperidin-1-yl)phenyl]thiophene-2-sulfonamide |
| 12 | N1,N1-dimethyl-N4-[2-(4-phenylpiperidin-1-yl)phenyl]benzene-1,4-disulfonamide |
| 13 | tert-butyl 4-{2-[4-(dimethylsulfamoyl)benzenesulfonamido]phenyl}piperazine-1-carboxylate |
| 14 | 1-(2-((4-(N,N-dimethylsulfamoyl)phenyl)sulfonamido)phenyl)-N-methyl-N-propylpiperidine-4-carboxamide |
| 15 | 4-(dimethylsulfamoyl)-N-methyl-N-[2-(piperidin-1-yl)phenyl]benzamide |
| 16 | N-(2-{2-azabicyclo[2.2.1]heptan-2-yl}phenyl)-5-tert-butylthiophene-2-sulfonamide |
| 17 | N-(2-{2-azabicyclo[2.2.1]heptan-2-yl}phenyl)-5-ethylthiophene-2-sulfonamide |
| 18 | tert-butyl 4-(2-((4-ethylphenyl)sulfonamido)phenyl)piperazine-1-carboxylate |
| 19 | tert-butyl 1-(2-((4-ethylphenyl)sulfonamido)phenyl)piperidine-4-carboxylate |
| 20 | tert-butyl 4-(2-(4-(N,N-dimethylsulfamoyl)-N-ethylbenzamido)phenyl)piperazine-1-carboxylate |
| 21 | N-(2-{2-azabicyclo[2.2.1]heptan-2-yl}phenyl)-5-ethyl-N-methylthiophene-2-sulfonamide |
| 22 | N1,N1-dimethyl-N4-[2-(piperidin-1-yl)cyclohexyl]benzene-1,4-disulfonamide |
| 23 | 3-methyl-N-[2-(piperidin-1-yl)phenyl]benzene-1-sulfonamide |
| 24 | N1,N1-dimethyl-N4-[2-(4-phenoxypiperidin-1-yl)phenyl]benzene-1,4-disulfonamide |
| 25 | N4-{2-[4-(benzyloxy)piperidin-1-yl]phenyl}-N1,N1-dimethylbenzene-1,4-disulfonamide |
| 26 | N4-[2-(4-methoxypiperidin-1-yl)phenyl]-N1,N1-dimethylbenzene-1,4-disulfonamide |
| 27 | N1,N1-dimethyl-N4-[2-(morpholin-4-yl)phenyl]benzene-1,4-disulfonamide |
| 28 | tert-butyl 4-(2-((4-isopropylphenyl)sulfonamido)phenyl)piperazine-1-carboxylate |
| 29 | tert-butyl 1-(2-((4-isopropylphenyl)sulfonamido)phenyl)piperidine-4-carboxylate |
| 30 | N1,N1-dimethyl-N4-(2-(piperidin-1-ylmethyl)phenyl)benzene-1,4-disulfonamide |
| 31 | tert-butyl 1-(2-((4-isopropoxyphenyl)sulfonamido)phenyl)piperidine-4-carboxylate |
| 32 | N1,N1-dimethyl-N4-[2-(pyrrolidin-1-yl)phenyl]benzene-1,4-disulfonamide |
| 33 | N4-[2-(4,4-difluoropiperidin-1-yl)phenyl]-N1,N1-dimethylbenzene-1,4-disulfonamide |
| 35 | N1,N1-dimethyl-N4-[2-(3-phenylpiperidin-1-yl)phenyl]benzene-1,4-disulfonamide |
| 36 | N4-[2-(3-methoxypiperidin-1-yl)phenyl]-N1,N1-dimethylbenzene-1,4-disulfonamide |
| 37 | N1,N1-dimethyl-N4-[2-(3-phenylpyrrolidin-1-yl)phenyl]benzene-1,4-disulfonamide |
| 38 | N1-(2-(4-hydroxypiperidin-1-yl)phenyl)-N4,N4-dimethylbenzene-1,4-disulfonamide |
| 39 | 4-isopropoxy-N-(2-(piperidin-1-yl)phenyl)benzenesulfonamide |
| 40 | N1-(2-(4-methoxyazepan-1-yl)phenyl)-N4,N4-dimethylbenzene-1,4-disulfonamide |
| 41 | N1-(2-(4-methoxyazepan-1-yl)phenyl)-N4,N4-dimethylbenzene-1,4-disulfonamide |
| 42 | N1-(2-(4-methoxyazepan-1-yl)phenyl)-N4,N4-dimethylbenzene-1,4-disulfonamide |
| 43 | N1-(2-(l,4-oxazepan-4-yl)phenyl)-N4,N4-dimethylbenzene-1,4-disulfonamide |
| 44 | N1-(2-(4-fluoropiperidin-1-yl)phenyl)-N4,N4-dimethylbenzene-1,4-disulfonamide |
| 45 | N1,N1-dimethyl-N4-(2-(4-propoxypiperidin-1-yl)phenyl)benzene-1,4-disulfonamide |
| 46 | tert-butyl 4-(2-(4-(N,N-dimethylsulfamoyl)-N-isopropylbenzamido)phenyl)piperazine-1-carboxylate |
| 47 | tert-butyl 4-(2-((2-methylphenyl)sulfonamido)phenyl)piperazine-1-carboxylate |
| 49 | N-(2-{2-azabicyclo[2.2.1]heptan-2-yl}phenyl)-4-(dimethylsulfamoyl)-N-methylbenzamide |
| 50 | tert-butyl 4-(2-((2,4-dimethylphenyl)sulfonamido)phenyl)piperazine-1-carboxylate |
| 51 | tert-butyl 4-(2-((3,4-dimethylphenyl)sulfonamido)phenyl)piperazine-1-carboxylate |
| 52 | methyl 4-{2-[4-(dimethylsulfamoyl)benzenesulfonamido]phenyl}piperazine-1-carboxylate |
| 53 | 2,2,2-trifluoroethyl4-{2-[4-(dimethylsulfamoyl)benzenesulfonamido]phenyl}piperazine-1-carboxylate |
| 54 | tert-butyl 4-(2-((3,5-dimethylphenyl)sulfonamido)phenyl)piperazine-1-carboxylate |
| 55 | N1,N1-dimethyl-N4-[2-(4-phenylpiperidin-1-yl)cyclohexyl]benzene-1,4-disulfonamide |
| 56 | tert-butyl 4-(2-((2,4-dimethylthiazole)-5-sulfonamido)phenyl)piperazine-1-carboxylate |
| 57 | N1,N1-dimethyl-N4-[2-(4-phenoxypiperidin-1-yl)cyclohexyl]benzene-1,4-disulfonamide |
| 58 | tert-butyl 4-(2-((2,5-dimethylfuran)-3-sulfonamido)phenyl)piperazine-1-carboxylate |
| 59 | tert-butyl 4-(2-((2,5-dimethylthiophene)-3-sulfonamido)phenyl)piperazine-1-carboxylate |
| 60 | tert-butyl 4-(2-((5-methylpyridine)-3-sulfonamido)phenyl)piperazine-carboxylate |
| 61 | N1,N1-dimethyl-N4-[2-(morpholin-4-yl)cyclohexyl]benzene-1,4-disulfonamide |
| 62 | N1,N1-dimethyl-N4-[2-(4-phenoxypiperidin-1-yl)cyclohexyl]benzene-1,4-disulfonamide |
| 63 | N1,N1-dimethyl-N4-[2-(4-phenoxypiperidin-1-yl)cyclohexyl]benzene-1,4-disulfonamide |
| 64 | tert-butyl 4-(2-((3,5-dimethylisoxazole)-4-sulfonamido)phenyl)piperazine-1-carboxylate |
| 65 | 2,4-dimethyl-N-(2-(4-(pyridin-2-yloxy)piperidin-1-yl)phenyl)benzenesulfonamide |
| 66 | 2,4-dimethyl-N-(2-(4-propoxypiperidin-1-yl)phenyl)benzenesulfonamide |
| 67 | 2,5-dimethyl-N-(2-(4-propoxypiperidin-1-yl)phenyl)furan-3-sulfonamide |
| 68 | 2,5-dimethyl-N-(2-(4-propoxypiperidin-1-yl)phenyl)thiophene-3-sulfonamide |
| 70 | ethyl4-{2-[4-(dimethylsulfamoyl)benzenesulfonamido]phenyl}piperazine-1-carboxylate |
| 71 | N1,N1-dimethyl-N4-[2-(4-propanoylpiperazin-1-yl)phenyl]benzene-1,4-disulfonamide |
| 72 | tert-butyl 4-(2-((l-methylindoline)-5-sulfonamido)phenyl)piperazine-1-carboxylate |
| 73 | N4-[2-(4-benzoylpiperazin-1-yl)phenyl]-N1,N1-dimethylbenzene-1,4-disulfonamide |
| 74 | N1,N1-dimethyl-N4-{[2-(piperidin-1-yl)phenyl]methyl}benzene-1,4-disulfonamide |
| 75 | N1,N1-dimethyl-N4-{2-[4-(pyridin-2-yloxy)piperidin-1-yl]phenyl}benzene-1,4-disulfonamide |
| 76 | f-butyl 1-{2-[4-(dimethylsulfamoyl)benzenesulfonamido]phenyl}piperidine-4-carboxylate |
| 77 | N1,N1-dimethyl-N4-{2-[4-(pyrazin-2-yloxy)piperidin-1-yl]phenyl} benzene-1,4-disulfonamide |
| 78 | f-butyl 4-{2-[4-(dimethylsulfamoyl)benzenesulfonamido]phenyl} piperidine-1-carboxylate |
| 79 | N1,N1-dimethyl-N4-{2-[4-(thiophene-2-carbonyl)piperazin-1-yl]phenyl}benzene-1,4-disulfonamide |
| 80 | N1,N1-dimethyl-N4-{2-[4-(4-methyl-1,3-thiazole-5-carbonyl)piperazin-1-yl]phenyl}benzene-1,4-disulfonamide |
| 81 | N1,N1-dimethyl-N4-(2-{4-[2-(thiophen-2-yl)acetyl]piperazin-1-yl}phenyl)benzene-1,4-disulfonamide |
| 82 | 4-{2-[4-(dimethylsulfamoyl)benzenesulfonamido]phenyl}-N-methyl-N-propylpiperazine-1-carboxamide |
| 83 | t-butyl 4-[2-(4-methylbenzenesulfonamido)phenyl]piperazine-1-carboxylate |
| 84 | t-butyl 4-[2-(4-methoxybenzenesulfonamido)phenyl]piperazine-1-carboxylate |
| 85 | N4-[2-(azepan-1-yl)phenyl]-N 1 ,N1-dimethylbenzene-1,4-disulfonamide |
| 86 | N1,N1-dimethyl-N4-{2-[4-(pyridin-3-yloxy)piperidin-1-yl]phenyl}benzene-1,4-disulfonamide |
| 87 | 1-{2-[4-(dimethylsulfamoyl)benzenesulfonamido]phenyl}-N-propylpiperidine-4-carboxamide |
| 88 | 1-{2-[4-(dimethylsulfamoyl)benzenesulfonamido]phenyl}-N-(2-methylpropyl)piperidine-4-carboxamide |
| 89 | N1,N1-dimethyl-N4-{2-[4-(pyrimidin-2-yloxy)piperidin-1-yl]phenyl{benzene-1,4-disulfonamide |
| 90 | l-[2-(4-methylbenzenesulfonamido)phenyl]piperidin-4-yl |
| | 2,2-dimethylpropanoate |
| 91 | l-[2-(4-methoxybenzenesulfonamido)phenyl]piperidin-4-yl |
| | 2,2-dimethylproanoate |
| 92 | tert-butyl 4-(2-((2-methyl-2,3-dihydrobenzofuran)-5-sulfonamido)phenyl)piperazine-1-carboxylate |
| 93 | tert-butyl 4-(2-((2-methyl-2,3-dihydrobenzofuran)-5-sulfonamido)phenyl)piperazine-1-carboxylate |
| 94 | tert-butyl 4-(2-((2-methyl-2,3-dihydrobenzofuran)-5-sulfonamido)phenyl)piperazine-1-carboxylate |
| 95 | tert-butyl 4-(2-((8-methylquinoline)-5-sulfonamido)phenyl)piperazine-1-carboxylate |
| 96 | tert-butyl 4-(2-(N-isopropyl-4-(methylsulfonyl)benzamrdo)phenyl)piperazine-1-carboxylate |
| 97 | 2,5-dimethyl-N-(2-(4-(pyridin-2-yloxy)piperidin-1-yl)phenyl)thiophene-3-sulfonamide |
| 98 | 6-methyl-N-(2-(4-(pyridin-2-yloxy)piperidin-1-yl)phenyl)pyridine-3-sulfonamide |
| 99 | 2,5-dimethyl-N-(2-(4-(pyridin-2-yloxy)piperidin-1-yl)phenyl)thiazole-4-sulfonamide |
| 100 | N1,N1-dimethyl-N4-(2-(2-phenylmorpholino)phenyl)benzene-1,4-disulfonamide |
| 101 | N1-(2-(2-benzylmorpholino)phenyl)-N4,N4-dimethylbenzene-1,4-disulfonamide |
| 103 | N-isopropyl-4-(methylsulfonyl)-N-(2-(4-(pyrazin-2-yloxy)piperidin-1-yl)phenyl)benzamide |
| 105 | methyl 1-(2-((4-(N,N-dimethylsulfamoyl)phenyl)sulfonamido)phenyl)piperidine-4-carboxylate |
| 106 | N1,N1-dimethyl-N4-(2-(4-(pyridin-4-yloxy)piperidin-1-yl)phenyl)benzene-1,4-disulfonamide |

Also provided herein is a compound as disclosed herein for use as a medicament.

Also provided herein is a compound as disclosed herein, or a stereoisomer thereof, or a salt of any of the foregoing, for use in the treatment of a TRPML1-mediated disease.

Also provided herein is a compound as disclosed herein, or a stereoisomer thereof, or a salt of any of the foregoing, for use in the manufacture of a medicament for the prevention or treatment of a disease or condition ameliorated by the modulation of TRPML1.

Also provided herein is a pharmaceutical composition comprising a compound as disclosed herein, or a stereoisomer thereof, or a salt of any of the foregoing, together with a pharmaceutically acceptable carrier.

Also provided herein is a method of treatment of a TRPML1-mediated disease comprising the administration of a therapeutically effective amount of a compound as disclosed herein, or a stereoisomer thereof, or a salt of any of the foregoing, to a subject in need thereof.

In certain embodiments, the disease is chosen from cancer, an inflammatory disorder, pain, a neurodegenerative disorder, a cognitive disorder, and a psychiatric disorder.

In certain embodiments, the disease is Alzheimer's Disease.

Also provided herein is a method of modulation of TRPML1 comprising contacting

TRPML1 with a compound as disclosed herein, or a stereoisomer thereof, or a salt of any of the foregoing.

Also provided herein is a method of making a compound disclosed herein, comprising the procedure(s) described below and variations thereupon.

Compounds of formula (I) can be prepared by reacting a compound of formula (II) or formula (III):

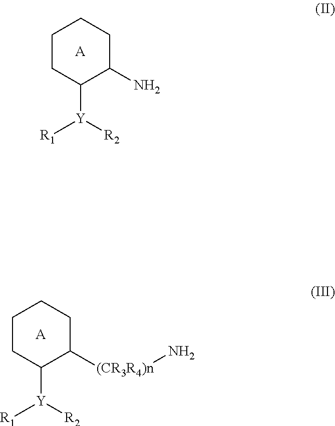

Wherein Y, R1, R2, R3, R4 and n are as defined above, with a compound of formula (IV) or formula (V)

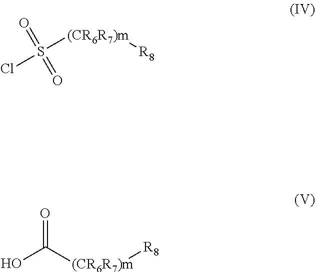

Wherein R6, R7, m and R8 are as defined above.

The reaction of a compound of formula (II) or formula (III) with a compound of formula (IV) may be carried out in a reaction-inert solvent such as DCM or THF, and in the presence of a suitable base such as TEA or pyridine. The reaction may conveniently be carried out at temperatures between room temperature and the reflux temperature of the reaction mixture and optionally converting the obtained compound of formula (I) into an addition salt thereof, and/or preparing stereochemically isomeric forms thereof.

The reaction of compound of formula (II) or formula (III) with a compound of formula (V) may be carried out in a reaction-inert solvent such as DCM, and in the presence of an activator such as HATU at room temperature.

Reagents of formula (IV) and formula (V) are known in the art.

Reagents of formula (II) or formula (III) either are commercially available, or can be prepared according to the following schemes:

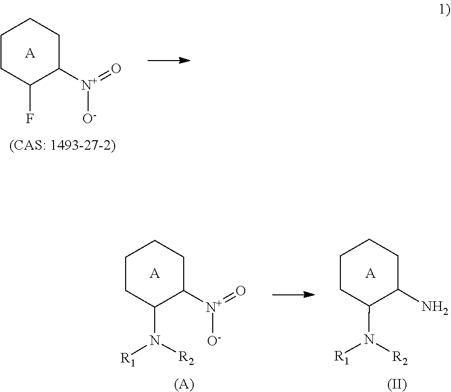

Wherein A, R1 and R2 are as defined above;

Compounds of formula (I) can also be prepared by reacting a compound of formula (VI):

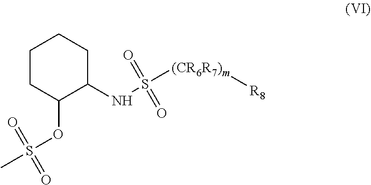

Wherein R₆, R₇, m and R₈ are as defined above; with a suitable amine, optionally substituted. The reaction may be carried out in a reaction-inert solvent such as DCM or THF, and in the presence of a suitable base such as TEA at room temperature.

Compounds of formula (VI) can be prepared according to the following schemes:

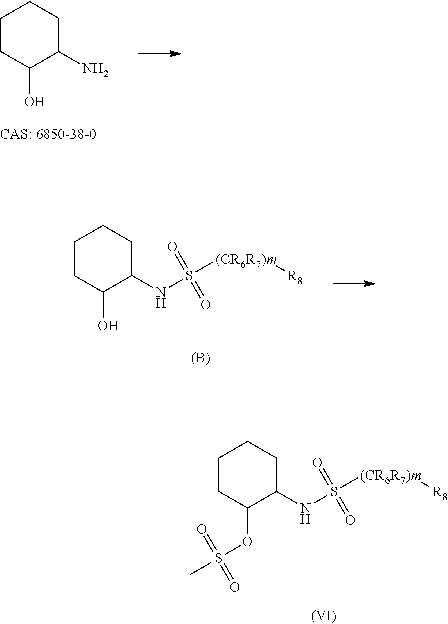

Compounds of formula (I) may also be prepared for reaction of compound of formula (VII) or compound of formula (VIII) with CH₃I or CH₃CH₂I or (CH₃)₂CHI;

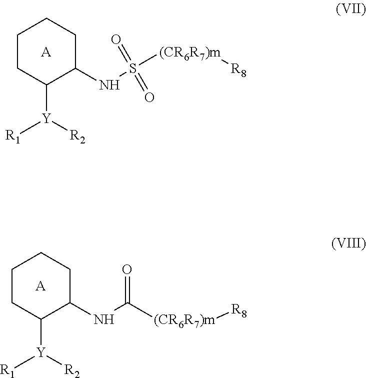

Wherein A, Y, R₁, R₂, R₆, R₇, m and R₈ are as defined above. The reaction may be carried out in a reaction-inert solvent such as DMF, and in the presence of a suitable base such as NaH or K₂CO₃ at room temperature or heated to 100° C.

Compounds of formula (VII) can be prepared according to the previous synthetic scheme.

Compound of formula (VIII) can be prepared according to the following synthetic scheme:

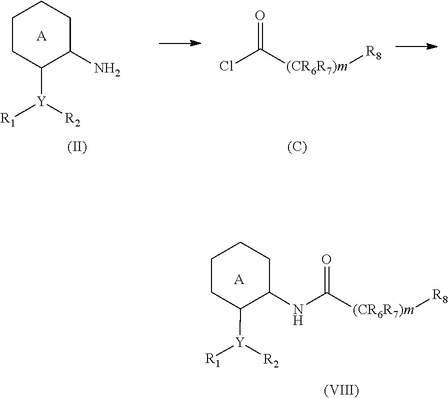

Compounds of formula (II) are commercially available or can be prepared according to the previous scheme.

Compounds of formula (C) are known in the art.

Compounds of formula (I) may also be prepared for reaction of compound of formula (IX)

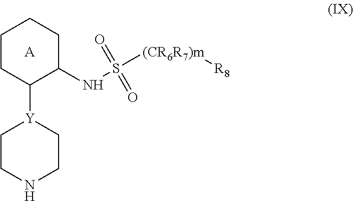

Wherein A, Y, R₆, R₇, m and R₈ are as defined above, with a suitable compounds of formula (X)

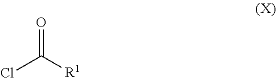

Wherein R₁ is as defined above. This reaction may be carried out in a reaction-inert solvent such as DCM or THF, and in the presence of a suitable base such as TEA at room temperature.

Compounds of formula (X) are known in the art.

Compounds of formula (IX) can be prepared according to the following scheme:

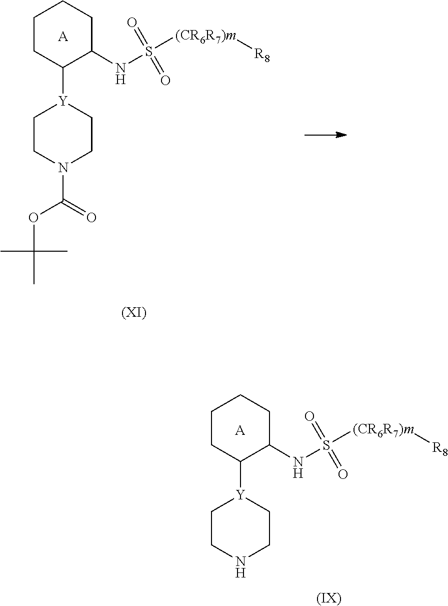

Compounds of formula (I) may also be prepared for reaction of compound of formula (XII)

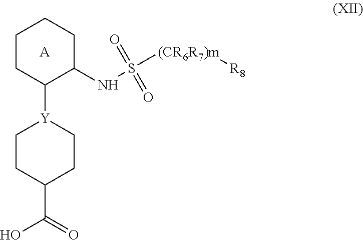

Wherein A, Y, R6, R7, m and R8 are as defined above, with a suitable compounds of formula (XIII)

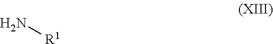

Wherein R1 is as defined above. This reaction may be carried out in a reaction-inert solvent such as DCM, and in the presence of an activator such as HATU at room temperature.

Compounds of formula (XII) can be prepared according to the previous schemes.

Compounds of formula (XIII) are known in the art.

The compounds disclosed herein may be synthesized as enantiomers or in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated there from by alkali. An alternative manner of separating the enantiomeric forms of the compounds of formula (1) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

The compounds of formula (I), the pharmaceutically acceptable salts and stereoisomeric forms thereof possess TRPML1 receptor agonism as demonstrated in the Pharmacological Examples. The compounds of formula (I) as prepared in the hereinabove described processes may be synthesized in the form of racemic mixtures of enantiomers which can be separated from one another following art-known resolution procedures. Those compounds of formula (I) that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated there from by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials. In the preparation of the compounds of formula I and the starting materials and/or intermediates described herein it may be useful to protect certain groups which are sensitive to the reaction conditions. The evaluation of the usefulness of the optional protection, as well as the selection of the suitable protecting agent, according to the reaction carried out in the preparation of the compounds provided herein and the functional group to be protected, are within the common knowledge of the skilled person. The removal of the optional protective groups is carried out according to conventional techniques.

The preparation of the salts of the compounds of formula I is carried out according to known methods.

The present compounds of formula (I) are useful in the treatment of a condition or disease mediated by the modulation of the function of the TRPML1 receptor, in particular TRPML1 receptor agonistic activity, or Positive Allosteric Modulation. Furthermore, the present compounds may be used for the manufacture of a medicine for treatment of a condition or a disease mediated by TRPML1 receptor activity, in particular TRPML1 receptor agonistic activity.

The present disclosure also provides the use of a compound of formula (I) or a pharmaceutically acceptable salt thereof for the manufacture of a medicament for the treatment of conditions or diseases selected from TRPML1 receptor mediated conditions or diseases.

### Definitions

As used in the foregoing definitions:

The term “stereochemically isomeric forms” as used hereinbefore defines all the possible isomeric forms which the compounds of formula (I) may possess. Unless otherwise mentioned or indicated, the chemical designation of compounds denotes the mixture of all possible stereochemically isomeric forms, said mixtures containing all diastereomers and enantiomers of the basic molecular structure. More in particular, stereogenic centers may have the R- or S-configuration; substituents on bivalent cyclic (partially) saturated radicals may have either the cis- or trans-configuration.

Stereochemically isomeric forms of the compounds of formula (I) are obviously intended to be embraced within the scope of this invention.

The absolute stereochemical configuration of the compounds of formula (I) and of the intermediates used in their preparation may easily be determined by those skilled in the art while using well-known methods such as, for example, X-ray diffraction.

Furthermore, some compounds of formula (I) and some of the intermediates used in their preparation may exhibit polymorphism. It is to be understood that the present invention encompasses any polymorphic forms possessing properties useful in the treatment of the conditions noted herein above.

The pharmaceutically acceptable salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, trifluoromethanesulfonic, ethanesulfonic, benzenesulfonic, p-toluenesulfonic, cyclamic, salicylic, p-aminosalicylic, pamoic and the like.

Conversely said salt forms can be converted by treatment with an appropriate base into the free base form.

The compounds of formula (I) may exist in both unsolvated and solvated forms. The term ‘solvate’ is used herein to describe a molecular association comprising a compound of the invention and one or more pharmaceutically acceptable solvent molecules, e.g. water or ethanol. The term ‘hydrate’ is used when said solvent is water.

As used herein, the term “about” is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

As used herein, the term “agonist” refers to a moiety that interacts with, and activates, a receptor and thereby initiates a physiological or pharmacological response characteristic of that receptor.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease, for example as in the case of prevention of infection with a pathogen, or may involve prevention of disease progression, for example from prediabetes to diabetes. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

Pharmaceutical Compositions

Additionally, pharmaceutical compositions/formulations comprising at least one pharmaceutically acceptable carrier and a therapeutically effective amount of a compound of formula (I) are provided herein.

In order to prepare the pharmaceutical compositions of the compounds provided herein, an effective amount of the particular compound, optionally in base or acid addition salt form, as the active ingredient, is combined with at least one pharmaceutically acceptable carrier, which carrier may take a variety of forms depending on the form of preparation desired for administration. These pharmaceutical compositions may be in unitary dosage form suitable for oral administration, rectal administration, percutaneous administration or parenteral injection.

For example in preparing the compositions in oral dosage form, any of the usual liquid pharmaceutical carriers may be employed, such as for instance water, glycols, oils, alcohols and the like in the case of oral liquid preparations such as suspensions, syrups, elixirs and solutions; or solid pharmaceutical carriers such as starches, sugars, kaolin, lubricants, binders, disintegrating agents and the like in the case of powders, pills, capsules and tablets. Because of their easy administration, tablets and capsules represent the most advantageous oral dosage unit form, in which case solid pharmaceutical carriers are obviously employed. For parenteral injection compositions, the pharmaceutical carrier will mainly comprise sterile water, although other ingredients may be included in order to improve solubility of the active ingredient.

Injectable solutions may be prepared for instance by using a pharmaceutical carrier comprising a saline solution, a glucose solution or a mixture of both. Injectable suspensions may also be prepared by using appropriate liquid carriers, suspending agents and the like. In compositions suitable for percutaneous administration, the pharmaceutical carrier may optionally comprise a penetration enhancing agent and/or a suitable wetting agent, optionally combined with minor proportions of suitable additives which do not cause a significant deleterious effect to the skin. Said additives may be selected in order to facilitate administration of the active ingredient to the skin and/or be helpful for preparing the desired compositions. These topical compositions may be administered in various ways, e.g., as a transdermal patch, a spot-on or an ointment. Addition salts of the compounds of formula (1), due to their increased water solubility over the corresponding base form, are obviously more suitable in the preparation of aqueous compositions.

It is especially advantageous to formulate the pharmaceutical compositions of the compounds disclosed herein in dosage unit form for ease of administration and uniformity of dosage.

“Dosage unit form” as used herein refers to physically discrete units suitable as unitary dosages, each unit containing a predetermined amount of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

For oral administration, the pharmaceutical compositions of the compounds disclosed herein may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, tale, silica and the like), disintegrating agents (e.g. potato starch, sodium starch glycollate and the like), wetting agents (e.g. sodium lauryl sulphate) and the like. Such tablets may also be coated by methods well known in the art.

Liquid preparations for oral administration may take the form of e.g. solutions, syrups or suspensions, or they may be formulated as a dry product for admixture with water and/or another suitable liquid carrier before use. Such liquid preparations may be prepared by conventional means, optionally with other pharmaceutically acceptable additives such as suspending agents (e.g. sorbitol syrup, methylcellulose, hydroxypropylmethylcellulose or hydrogenated edible fats), emulsifying agents (e.g. lecithin or acacia), non-aqueous carriers (e.g. almond oil, oily esters or ethyl alcohol), sweeteners, flavours, masking agents and preservatives (e.g. methyl or propyl p-hydroxybenzoates or sorbic acid).

Pharmaceutically acceptable sweeteners useful in the pharmaceutical compositions of the disclosure comprise preferably at least one intense sweetener such as aspartame, acesulfame potassium, sodium cyclamate, alitarne, dihydrochalcone sweetener, monellin, stevioside sucralose (4,1′,6′-trichloro-4,1′,6′-trideoxygalactosucrose) or, preferably, saccharin, sodium or calcium saccharin, and optionally at least one bulk sweetener such as sorbitol, mannitol, fructose, sucrose, maltose, isomalt, glucose, hydrogenated glucose syrup, xylitol, caramel or honey. Intense sweeteners are conveniently used in low concentrations. For example, in the case of sodium saccharin, the said concentration may range from about 0.04% to 0.1% (weight/volume) of the final formulation. The bulk sweetener can effectively be used in larger concentrations ranging from about 10% to about 35%, preferably from about 10% to 15% (weight/volume). The pharmaceutically acceptable flavours which can mask the bitter tasting ingredients in the low-dosage formulations comprise preferably fruit flavours such as cherry, raspberry, black currant or strawberry flavour. A combination of two flavours may yield very good results. In the high-dosage formulations, stronger pharmaceutically acceptable flavours may be required such as Caramel Chocolate, Mint Cool, Fantasy and the like.

Each flavour may be present in the final composition in a concentration ranging from about 0.05% to 1% (weight/volume). Combinations of said strong flavours are advantageously used. Preferably a flavour is used that does not undergo any change or loss of taste and/or color under the circumstances of the formulation.

The compounds of formula (I) may be formulated for parenteral administration by injection, conveniently intravenous, intra-muscular or subcutaneous injection, for example by bolus injection or continuous intravenous infusion. Formulations for injection may be presented in unit dosage form, e.g. in ampoules or multi-dose containers, including an added preservative. They may take such forms as suspensions, solutions or emulsions in oily or aqueous vehicles, and may contain formulating agents such as isotonizing, suspending, stabilizing and/or dispersing agents. Alternatively, the active ingredient may be present in powder form for mixing with a suitable vehicle, e.g. sterile pyrogen-free water, before use.

The compounds of formula (I) may also be formulated in rectal compositions such as suppositories or retention enemas, e.g. containing conventional suppository bases such as cocoa butter and/or other glycerides.

Those of skill in the treatment of diseases linked to the mediation of the ligand-gated ion channels will easily determine the therapeutically effective amount of a compound of formula (I) from the test results presented hereinafter. In general, it is contemplated that a therapeutically effective dose will be from about 0.001 mg/kg to about 50 mg/kg of body weight, more preferably from about 0.01 mg/kg to about 10 mg/kg of body weight of the patient to be treated. It may be appropriate to administer the therapeutically effective dose in the form of two or more sub-doses at appropriate intervals throughout the day. Said sub-doses may be formulated as unit dosage forms, for example each containing from about 0.1 mg to about 1000 mg, more particularly from about 1 to about 500 mg, of the active ingredient per unit dosage form.

As used herein, a “therapeutically effective amount” of a compound, is the quantity of a compound which, when administered to an individual or animal, results in a sufficiently high level of that compound in the individual or animal to cause a discernible TRPML1 modulating response.

The exact dosage and frequency of administration depends on the particular compound of formula (I) used, the particular condition being treated, the severity of the condition being treated, the age, weight and general physical condition of the particular patient as well as the other medication, the patient may be taking, as is well known to those skilled in the art. Furthermore, said “therapeutically effective amount” may be lowered or increased depending on the response of the treated patient and/or depending on the evaluation of the physician prescribing the compounds of the instant invention. The effective daily amount ranges mentioned hereinabove are therefore only guidelines.

Indications and Methods of Treatment

Also provided herein are methods for treating TRPML1-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. Certain embodiments provide therapeutic compositions comprising at least one compound disclosed herein in combination with one or more additional agents for the treatment of TRPML1-mediated disorders.

Also provided herein are: the use of a compound of formula (I) or a pharmaceutically acceptable salt thereof as a medicament; the use of a compound of formula (I) or a pharmaceutically acceptable salt thereof in, or in the treatment of, a TRPML1 mediated disease; the use of a compound of formula (I) or a pharmaceutically acceptable salt thereof in the manufacture of a medicament for the treatment of a TRPML1 mediated disease; and a method of treatment of TRPML1 mediated disease comprising administering a compound of formula (I) or a pharmaceutically acceptable salt thereof. Further provided herein is a method of treatment of a disease mediated by TRPML1 activity, in a mammalian subject, which comprising administering a therapeutically effective amount of a compound of formula (I) or a pharmaceutically acceptable salt thereof.

TRPML1-mediated diseases include proliferative disorders such as cancers,

inflammatory disorders, pain, neurodegenerative disorders, cognitive and psychiatric disorders, and other diseases as disclosed below.

Compounds disclosed herein are useful for the treatment of neurodegenerative disorders of various origins such as Alzheimer's disease and other dementia conditions such as Lewy body dementia, fronto-temporal dementia and other taupathies; amyotrophic lateral sclerosis, multiple sclerosis, Parkinson's disease and other parkinsonian syndromes; Huntington's disease; HIV-induced neuroinflammation; essential tremors; other spinocerebellar degenerations, neuropathies such as Charcot-Marie-Tooth neuropathy and other TRPML1-mediated diseases such as Type IV mucolipidosis (MLIV). The compounds disclosed herein are also useful for the treatment of neurological conditions such as epilepsy including simple partial seizure, complex partial seizure, secondary generalized seizure, further including absence seizure, myoclonic seizure, clonic seizure, tonic seizure, tonic clonic seizure and atonic seizure, and for prevention and treatment of status epilepticus (SE).

The compounds disclosed herein are also useful for the treatment of cognitive disorders and of psychiatric disorders. Psychiatric disorders include, and are not limited to major depression, dysthymia, mania, bipolar disorder (such as bipolar disorder type I, bipolar disorder type II), cyclothymic disorder, rapid cycling, ultradian cycling, mania, hypomania, schizophrenia, schizophreniform disorders, schizoaffective disorders, personality disorders, attention disorders with or without hyperactive behaviour, delusional disorders, brief psychotic disorders, shared psychotic disorders, psychotic disorder due to a general medical condition, substance-induced psychotic disorders or a psychotic disorder not otherwise specified, anxiety disorders such as generalised anxiety disorder, panic disorders, posttraumatic stress disorder, impulse control disorders, phobic disorders, dissociative states and moreover in smoke, drug addiction and alcoholism. In particular bipolar disorders, psychosis, anxiety and addiction.

The compounds disclosed herein are useful in the prevention or treatment of neuroinflammation and CNS damage induced by HIV infection and of HIV-associated neurocognitive deficits. The compounds disclosed herein are useful in the prevention or treatment of neuropathic pain. Neuropathic pain syndromes include, and are not limited to: chemotherapy-induced peripheral neuropathy, diabetic neuropathy; sciatica; non-specific lower back pain; multiple sclerosis pain; fibromyalgia; HIV-related neuropathy; neuralgia, such as post-herpetic neuralgia and trigeminal neuralgia, Morton's neuralgia, causalgia; and pain resulting from physical trauma, amputation, phantom limb, cancer, toxins or chronic inflammatory conditions; central pain such as the one observed in thalamic syndromes, mixed central and peripheral forms of pain such as complex regional pain syndromes (CRPS) also called reflex sympathetic dystrophies.

The compounds disclosed herein are also useful for the treatment of pain, including chronic pain. Chronic pain includes, and is not limited to, chronic pain caused by inflammation or an inflammatory-related condition, ostheoarthritis, rheumatoid arthritis, acute injury or trauma, upper back pain or lower back pain (resulting from systematic, regional or primary spine disease such as radiculopathy), bone pain (due to osteoarthritis, osteoporosis, bone metastasis or unknown reasons), pelvic pain, spinal cord injury-associated pain, cardiac chest pain, non-cardiac chest pain, central post-stroke pain, myofascial pain, sickle cell pain, cancer pain, Fabry's disease, AIDS pain, geriatric pain or pain caused by headache, temporomandibular joint syndrome, gout, fibrosis or thoracic outlet syndromes, in particular rheumatoid arthritis and osteoarthritis.

The compounds disclosed herein are also useful in the treatment of acute pain caused by acute injury, illness, sport-medicine injuries, carpal tunnel syndrome, burns, musculoskeletal sprains and strains, musculotendinous strain, cervicobrachial pain syndromes, dyspepsia, gastric ulcer, duodenal ulcer, dysmenorrhea, endometriosis or surgery (such as open heart or bypass surgery), post-operative pain, kidney stone pain, gallbladder pain, gallstone pain, obstetric pain or dental pain.

The compounds disclosed herein are also useful in the treatment of headaches such as migraine, tension type headache, transformed migraine or evolutive headache, cluster headache, as well as secondary headache disorders, such as the ones derived from infections, metabolic disorders or other systemic illnesses and other acute headaches, paroxysmal hemicrania and the like, resulting from a worsening of the above mentioned primary and secondary headaches.

Compounds disclosed herein are also useful in the treatment of diseases such as vertigo, tinnitus, muscle spasm, and other disorders including and not limited to cardiovascular diseases (such as cardiac arrhythmia, cardiac infarction or angina pectoris, hypertension, cardiac ischemia, cerebral ischemia) endocrine disorders (such as acromegaly or diabetes insipidus) diseases in which the pathophysiology of the disorder involves excessive or hypersecretory or otherwise inappropriate cellular secretion of an endogenous substance (such as catecholamine, a hormone or a growth factor).

The compounds disclosed herein are also useful in the selective treatment of liver disease, such as inflammatory liver diseases, for example chronic viral hepatitis B, chronic viral hepatitis C, alcoholic liver injury, primary biliary cirrhosis, autoimmune hepatitis, liver fibrosis, non-alcoholic steatohepatitis and liver transplant rejection.

The compounds disclosed herein inhibit inflammatory processes affecting all body systems. Therefore, they are useful in the treatment of inflammatory processes of the muscularskeletal system of which the following is a list of examples but it is not comprehensive of all target disorders: arthritic conditions such as ankylosing spondylitis, cervical arthritis, fibromyalgia, gout, juvenile rheumatoid arthritis, lumbosacral arthritis, osteoarthritis, osteoporosis, psoriatic arthritis, rheumatic disease; disorders affecting skin and related tissues: eczema, psoriasis, dermatitis and inflammatory conditions such as sunburn; disorders of the respiratory system: asthma, allergic rhinitis and respiratory distress syndrome, lung disorders in which inflammation is involved such as asthma and bronchitis; chronic obstructive pulmonary disease; disorders of the immune and endocrinological systems: periarthritis nodosa, thyroiditis, aplastic anaemia, scleroderma, myasthenia gravis, multiple sclerosis and other demyelinating disorders, encephalomyelitis, sarcoidosis, nephritic syndrome, Bechet's syndrome, polymyositis, gingivitis.

Compounds disclosed herein are also useful in the treatment of gastrointestinal (GI) tract disorders such as inflammatory bowel disorders (IBD) including but not limited to ulcerative colitis, Crohn's disease, ileitis, proctitis, celiac disease, enteropathies, microscopic or collagenous colitis, eosinophilic gastroenteritis, or pouchitis resulting after proctocolectomy and post ileonatal anastomosis, and irritable bowel syndrome including any disorders associated with abdominal pain and/or abdominal discomfort such as pylorospasm, nervous indigestion, spastic colon, spastic colitis, spastic bowel, intestinal neurosis, functional colitis, mucous colitis, laxative colitis and functional dyspepsia; but also for treatment of atrophic gastritis, gastritis variolioforme, ulcerative colitis, peptic ulceration, pyrosis, and other damage to the GI tract, for example, by Helicobacter pylori, gastroesophageal reflux disease, gastroparesis, such as diabetic gastroparesis; and other functional bowel disorders, such as non-ulcerative dyspepsia (NUD); pancreatitis, emesis, diarrhoea, and visceral inflammation.

Compounds disclosed herein are also useful in the treatment of disorders of the genito-urinary tract such as overactive bladder, prostatitis (chronic bacterial and chronic nonbacterial prostatitis), prostadynia, interstitial cystitis, urinary incontinence and benign prostatic hyperplasia, annexities, pelvic inflammation, bartholinities and vaginitis. In particular, overactive bladder and urinary incontinence.

Compounds disclosed herein are also useful in the treatment of renal disorders including diabetic nephropathy, renal allograft rejection, infectious renal diseases, IgA nephropathy, fibrotic kidney disease, lupus nephritis and glomerulonephritis, acute kidney injury and renal carcinoma.

The compounds disclosed herein are also useful in the treatment of ophthalmic diseases such as retinitis, retinopathies, uveitis and acute injury to the eye tissue, age-related macular degeneration or glaucoma, conjunctivitis.

The compounds disclosed herein are also useful in the treatment of eating disorders such as anorexia nervosa including the subtypes restricting type and binge-eating/purging type; bulimia nervosa including the subtypes purging type and non-purging type; obesity; compulsive eating disorders; binge eating disorder, and eating disorder not otherwise specified.

The compounds disclosed herein are also useful in the treatment of allergic dermatitis, hyper-responsiveness of the airway, chronic obstructive pulmonary disease (COPD), bronchitis, septic shock, Sjögren's syndrome, glomerulonephritis, atherosclerosis, growth and metastases of malignant cells, myoblastic leukaemia, diabetes, meningitis, osteoporosis, burn injury, ischaemic heart disease, stroke, peripheral vascular disease, varicose veins, glaucoma.

In some embodiments, the compounds and pharmaceutical compositions of the present disclosure may be useful in the treatment or prevention of progression of cancer. The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL) Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma) and others as defined herein and known in the art.

Plasma cell malignancies include lymphoplasmacytic lymphoma, plasmacytoma, and multiple myeloma.

Solid tumors include melanomas, neuroblastomas, gliomas or 5 carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

General Synthetic Methods Nomenclature and Structures

In general, the nomenclature used in this Application is based on ChemSketch™ (ACDLabs) and generated according to the IUPAC systematic nomenclature. Chemical structures shown herein were prepared using ISIS® version 2.2. Certain compounds were drawn using CambridgeSoft's ChemDraw 18.0. Any open valency appearing on a carbon, oxygen, sulfur, or nitrogen atom in the structures herein indicates the presence of a hydrogen atom unless indicated otherwise. Where a nitrogen-containing heteroaryl ring is shown with an open valency on a nitrogen atom and variables such as R¹, R², R³ etc. are shown on the heteroaryl ring, such variables may be bound or joined to the open valency nitrogen. Where a chiral center exists in a structure, but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structure herein are intended to encompass all naturally occurring isotopes of such atoms. Thus, for example, the hydrogen atoms represented herein are meant to include deuterium and tritium, and the carbon atoms are meant to include ¹¹C, ¹³C and ¹⁴C isotopes and the fluorine atoms include ¹⁸F.

### ABBREVIATIONS

Abbreviations which are used in the description of the Schemes and the Examples that follows include:

ACN: Acetonitrile

AcOEt: Ethyl acetate

CH₃I: Iodomethane

CH₃CH₂I: Iodoethane

(CH₃)₂CHI: 2-Iodopropane

DCM: Dichloromethane

DMF: Dimethylformamide

DMSO: Dimethylsulfoxide

ESI: Electrospray ionization

h: hour

HATU: 1-[Bis(dimethylamino)methylene]-1H-1,2,3-triazolo[4,5-b]pyridinium-3-oxide hexafluorophosphate

K₂CO₃: Potassium carbonate

M: Molar

MeOH: Methanol

Min: Minute(s)

NMR: Nuclear Magnetic Resonance

NaH: Sodium hydride

NaOH: Sodium hydroxide

Na₂SO₄: Sodium sulfate

on: overnight

rt: Room Temperature

TFA: Trifluoroacetic acid

THF: Tetrahydrofuran

TEA: Triethylamine

UPLC-MS: UltraPerformance Liquid Chromatography-Mass Spectrometry

y: yield or yields.

### Experimental Part

The following examples illustrate the present invention. Unless explicitly stated otherwise, all measurements (especially percentages and amounts) relate to the weight.

### Synthesis of the Intermediates

Amines derivatives (II or III)

Most of amines derivatives (Compounds formula II or formula III) used, as starting materials, were purchased from chemical providers:

| Structure of amines | CAS number |
|---|---|
| 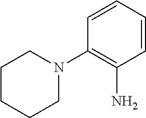 [SMILES: Nc1ccccc1N1CCCCC1] | 39643-31-7 |
| 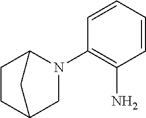 [SMILES: Nc1ccccc1N1CC2CCC1C2] | 1250695-37-4 |
| 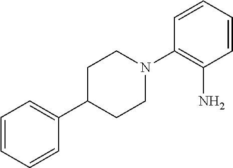 [SMILES: Nc1ccccc1N1CCC(c2ccccc2)CC1] | 252758-95-5 |
| 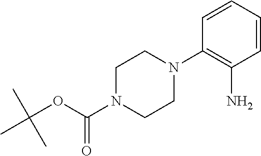 [SMILES: CC(C)(C)OC(=O)N1CCN(c2ccccc2N)CC1] | 170017-74-0 |
| 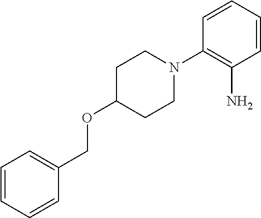 [SMILES: Nc1ccccc1N1CCC(OCc2ccccc2)CC1] | 1018636-14-0 |
| 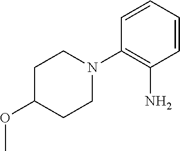 | 1018624-48-0 |
| 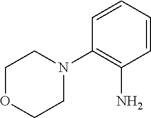 [SMILES: Nc1ccccc1N1CCOCC1] | 5585-33-1 |
| 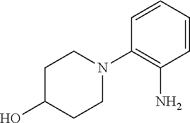 [SMILES: Nc1ccccc1N1CCC(O)CC1] | 252758-96-6 |
| 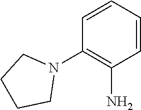 [SMILES: Nc1ccccc1N1CCCC1] | 21627-58-7 |
| 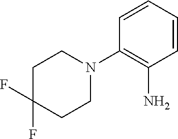 [SMILES: Nc1ccccc1N1CCC(F)(F)CC1] | 854044-39-6 |
| 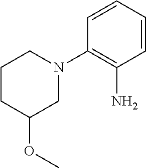 | 1602318-01-3 |
| 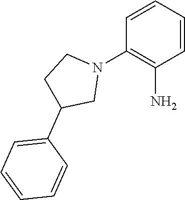 [SMILES: Nc1ccccc1N1CCC(c2ccccc2)C1] | 1272785-78-0 |
| 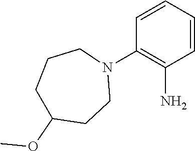 [SMILES: COC1CCCN(c2ccccc2N)CC1] | 1996133-97-1 |
| 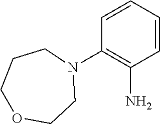 [SMILES: Nc1ccccc1N1CCCOCC1] | 955428-44-1 |
| 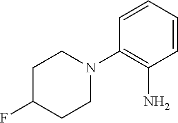 [SMILES: Nc1ccccc1N1CCC(F)CC1] | 1554044-58-4 |
| 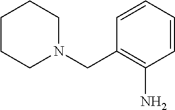 [SMILES: Nc1ccccc1CN1CCCCC1] | 19577-83-4 |
| 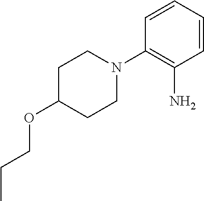 [SMILES: CCCOC1CCN(c2ccccc2N)CC1] | 1018624-51-5 |
| 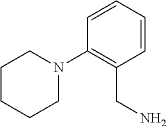 [SMILES: NCc1ccccc1N1CCCCC1] | 72752-54-6 |
| 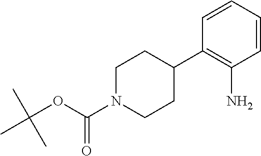 [SMILES: CC(C)(C)OC(=O)N1CCC(c2ccccc2N)CC1] | 199105-39-7 |
| 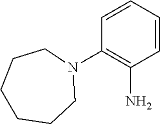 [SMILES: Nc1ccccc1N1CCCCCC1] | 51627-46-4 |
| 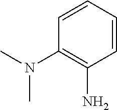 | 1262836-03-5 |
| 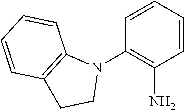 [SMILES: Nc1ccccc1N1CCc2ccccc21] | 180629-70-3 |
| 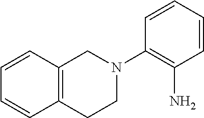 [SMILES: Nc1ccccc1N1CCc2ccccc2C1] | 246247-91-6 |

In the other cases, the amines derivatives (compounds of formula II) were synthesized according with the followed synthetic scheme:

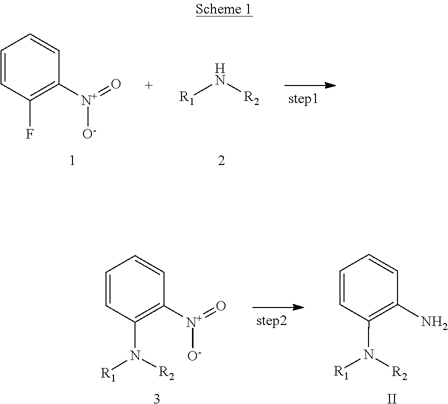

Step 1. To a solution of commercially available cmpd 1 (1.0 eq) in ACN (10 mL) cooled to 0° C., K₂CO₃ (3.0 eq) was added, followed by the addition of a suitable compound 2 (1.1 eq). The reaction mixture was stirred at rt on. Then the inorganic salts were filtered off and washed several times with THF. The filtrate was concentrated under reduced pressure. The residue was purified by flash silica gel column chromatography. In this way, the pure desired product 3 was obtained.

Step 2. To a solution of cmpd 3 (1.0 eq) in THF (4 mL) a solution of ammonium chloride (16.0 eq) in water (1 mL) and zinc (8.0 eq) were added sequentially and the mixture was stirred at rt on. The inorganic salts were filtered off and washed several times with THE. The filtrate was concentrated under reduced pressure and the residue diluted with AcOEt. The organic layer was washed with H₂O (×3), dried over Na₂SO₄, filtered and concentrated under reduced pressure. The residue was purified by flash silica gel column chromatography.

| Intermediate | Structure of amines | Yield |
|---|---|---|
| IIa | 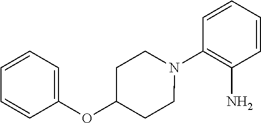 [SMILES: Nc1ccccc1N1CCC(Oc2ccccc2)CC1] | 85% |
| IIb | 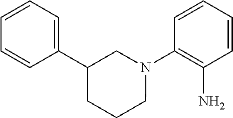 [SMILES: Nc1ccccc1N1CCCC(c2ccccc2)C1] | 94% |
| IIc | 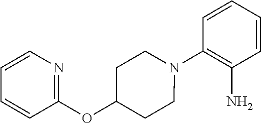 [SMILES: Nc1ccccc1N1CCC(Oc2ccccn2)CC1] | 80% |
| IId | 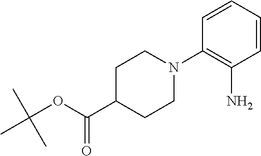 [SMILES: CC(C)(C)OC(=O)C1CCN(c2ccccc2N)CC1] | 97% |
| IIe | 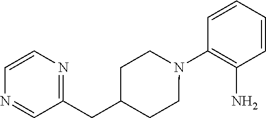 [SMILES: Nc1ccccc1N1CCC(Cc2cnccn2)CC1] | 75% |
| IIf | 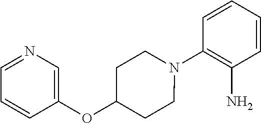 [SMILES: Nc1ccccc1N1CCC(Oc2cccnc2)CC1] | 81% |
| IIg | 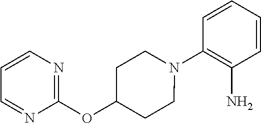 [SMILES: Nc1ccccc1N1CCC(Oc2ncccn2)CC1] | 74% |
| IIh | 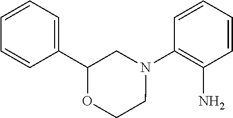 [SMILES: Nc1ccccc1N1CCOC(c2ccccc2)C1] | 87% |
| IIi | 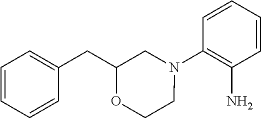 [SMILES: Nc1ccccc1N1CCOC(Cc2ccccc2)C1] | 77% |
| IIj | 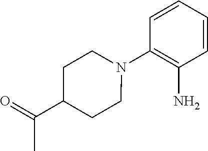 [SMILES: CC(=O)C1CCN(c2ccccc2N)CC1] | 61% |
| IIk | 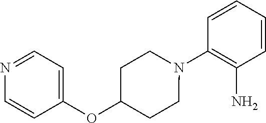 [SMILES: Nc1ccccc1N1CCC(Oc2ccncc2)CC1] | 30% |

Using these procedures, the following compounds II were synthesized:

Sulphonyl Chlorides (IV)

All the sulphonyl chlorides (Compounds formula IV) used as starting materials were purchased from cehmical providers:

| Structure of sulphonyl chlorides | CAS number |
|---|---|
| 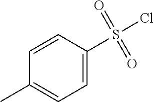 | 98-59-9 |
| 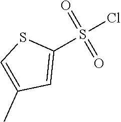 | 69815-97-0 |
| 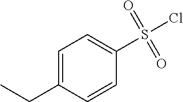 | 16712-69-9 |
| 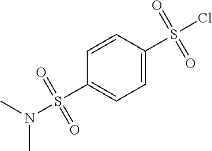 [SMILES: CN(C)S(=O)(=O)c1ccc(S(=O)(=O)Cl)cc1] | 677782-39-7 |
| 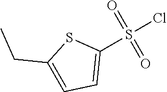 | 56921-00-7 |
| 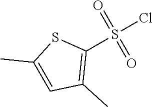 | 1314938-98-1 |
| 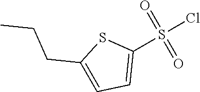 | 883156-08-5 |
| 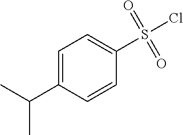 | 54997-90-9 |
| 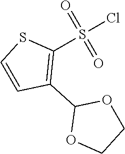 [SMILES: O=S(=O)(Cl)c1sccc1C1OCCO1] | 103011-38-7 |
| 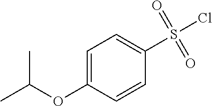 | 98995-40-5 |
| 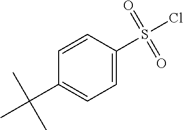 | 15084-51-2 |
| 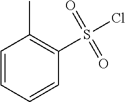 | 133-59-5 |
| 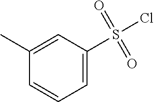 | 1899-93-2 |
| 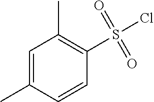 | 609-60-9 |
| 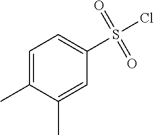 | 2905-30-8 |
| 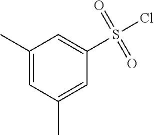 | 2905-27-3 |
| 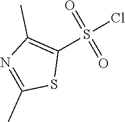 | 80466-80-4 |
| 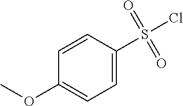 | 98-68-0 |
| 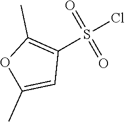 | 166964-26-7 |
| 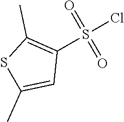 | 97272-04-3 |
| 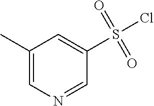 | 166337-57-1 |
| 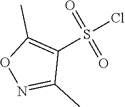 | 80466-79-1 |
| 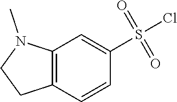 | 173669-61-9 |
| 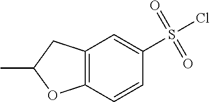 | 369638-66-4 |
| 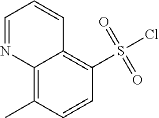 | 120164-05-8 |
| 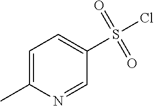 | 478264-00-5 |
| 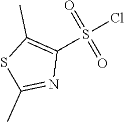 | 955085-16-2 |
| 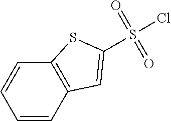 [SMILES: O=S(=O)(Cl)c1cc2ccccc2s1] | 90001-64-2 |
| 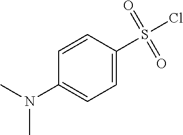 | 19715-49-2 |
| 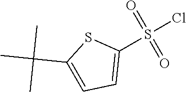 | 179400-17-0 |

Carboxylic Acids (V)

All the carboxylic acids (compounds formula V) used as starting materials were purchased from chemical providers:

| Structure of carboxylic acid | CAS number |
|---|---|
| 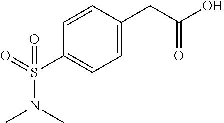 [SMILES: CN(C)S(=O)(=O)c1ccc(CC(=O)O)cc1] | 91013-44-4 |

Preparation of Intermediates of Formula (VI)

All the intermediates of formula VI were prepared according to the following synthetic scheme:

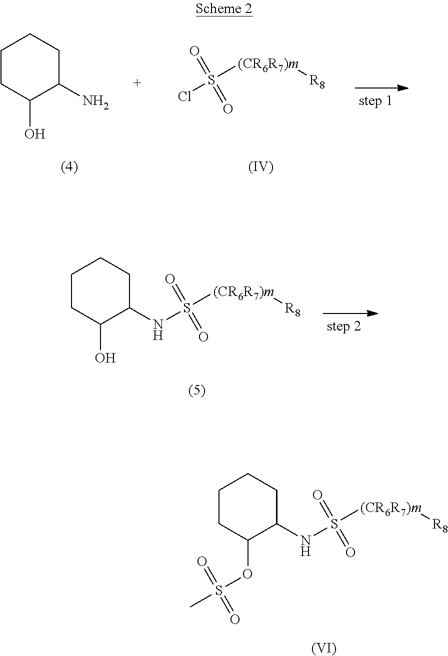

Step 1. To a solution of a suitable commercially available cmpd 4 (1.0 eq) in DCM (15 mL) cmpd formula IV (1.1 eq) and then TEA (3.0 eq) were added dropwise. The reaction was stirred at rt on. Then the reaction was partitioned with DCM and water, the organic layer was washed with brine, dried over Na₂SO₄ anhydrous, filtered and evaporated under reduced pressure. The crude residue was purified by flash silica gel column chromatography. In this way, the pure desired product 5 was obtained.

Step 2. To a cold (0° C.) solution of intermediate 5 (1.0 mL) in DCM (15.0 mL) methanesulfonyl chloride (1.2 eq) and then TEA (3.0 eq) were added. The reaction was stirred at rt for 2 hrs. Then the reaction was partitioned with DCM and water, the organic layer was washed with brine, dried over Na₂SO₄ anhydrous, filtered and evaporated under reduced pressure. The crude residue was used without purification in the next step. In this way the desired product VI was obtained.

Using the above procedure compound VIa was synthesized:

| Intermediate | Structure | Yield |
|---|---|---|
| Via | 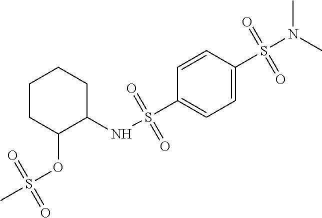 | 90% |

Preparation of Intermediates of Formula (VIII)

All the intermediates of formula VIII were prepared according to the following synthetic scheme:

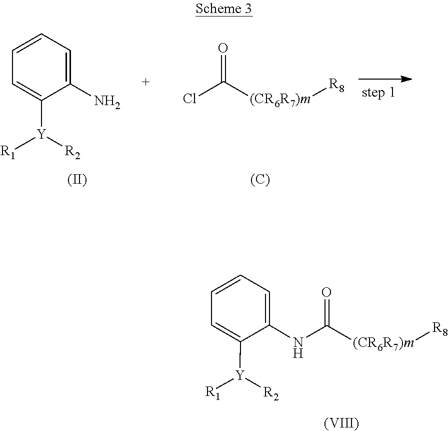

Step 1. A solution of a suitable commercially available cmpd of formula II (1.0 eq) in DCM (2 mL) was cooled to 0° C. and the appropriate acyl chloride C (1.1 eq) and pyridine or TEA (3.0 eq) were added dropwise. The reaction was stirred at rt on. Then the reaction was partitioned with DCM and water, the organic layer was washed with brine, dried over Na₂SO₄ anhydrous, filtered and evaporated under reduced pressure. The crude residue was purified by flash silica gel column chromatography.

In this way the desired product VIII was obtained.

Using the above procedure compounds VIII were synthesized:

| Intermediate | Structure | Yield |
|---|---|---|
| VIIIa | 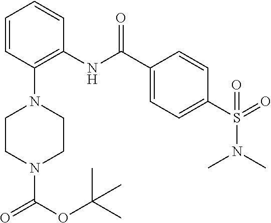 [SMILES: CN(C)S(=O)(=O)c1ccc(C(=O)Nc2ccccc2N2CCN(C(=O)OC(C)(C)C)CC2)cc1] | 50% |
| VIIIb | 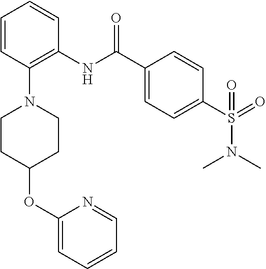 [SMILES: CN(C)S(=O)(=O)c1ccc(C(=O)Nc2ccccc2N2CCC(Oc3ccccn3)CC2)cc1] | 54% |
| VIIIc | 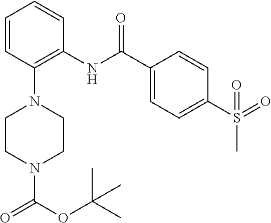 [SMILES: CC(C)(C)OC(=O)N1CCN(c2ccccc2NC(=O)c2ccc(S(C)(=O)=O)cc2)CC1] | 94% |
| VIIId | 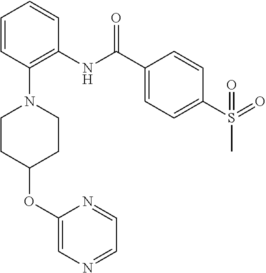 [SMILES: CS(=O)(=O)c1ccc(C(=O)Nc2ccccc2N2CCC(Oc3cnccn3)CC2)cc1] | 55% |
| VIIIe | 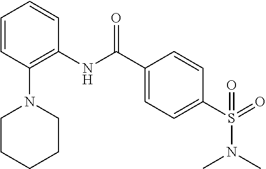 [SMILES: CN(C)S(=O)(=O)c1ccc(C(=O)Nc2ccccc2N2CCCCC2)cc1] | 81% |
| VIIIf | 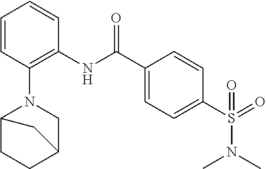 [SMILES: CN(C)S(=O)(=O)c1ccc(C(=O)Nc2ccccc2N2CC3CCC2C3)cc1] | 85% |

Acyl Chlorides (C)

All the acyl chlorides (compounds of formula C) used as starting materials were purchased from chemical providers:

| Structure of acyl chlorides | CAS number |
|---|---|
| 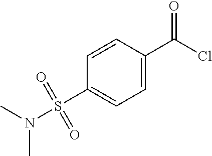 | 29171-70-8 |
| 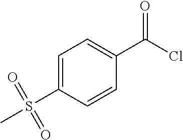 | 40913-92-6 |

Preparation of Intermediates of Formula (IX)

All the intermediates of formula IX were prepared according to the following synthetic scheme:

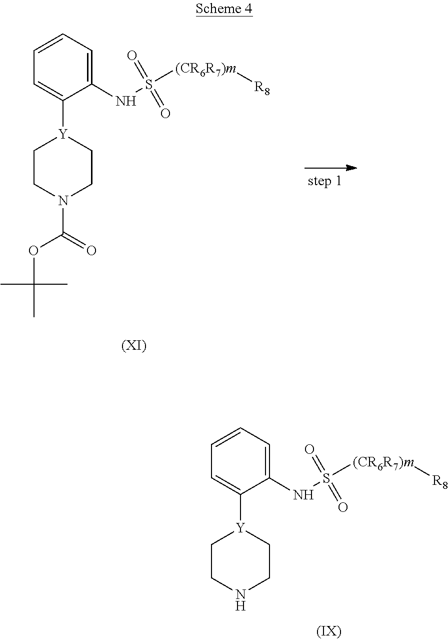

To a solution of a suitable compound of formula XI (1.0 eq) in DCM (5.0 mL), TFA (5.0 ml) was added and the reaction was stirred at rt for 2 hrs. Then the solvent was evaporated under reduced pressure, and the residue was taken up in DCM. The crude was used without purification in the next step.

Using these procedures compound IXa was synthesized:

| Intermediate | Structure | Yield |
|---|---|---|
| IXa | 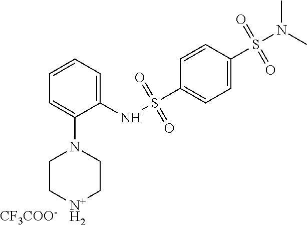 | 99% |

Preparation of Intermediates of Formula (XII)

All the intermediates of formula XII were prepared according to the following synthetic scheme:

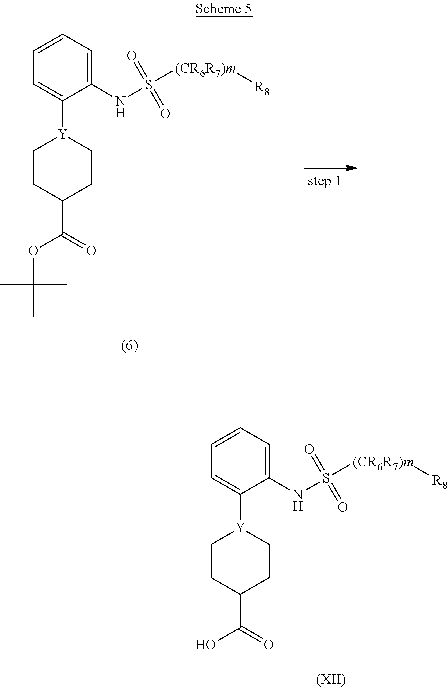

To a solution of a suitable compound of formula 6 (1.0 eq) in DCM (5.0 mL), TFA (5.0 ml) was added and the reaction was stirred at rt for 2 hrs. Then the solvent was evaporated under reduced pressure, and the residue was taken up in DCM. The crude was used without purification in the next step.

Using these procedures compound XIIa was synthesized:

| Intermediate | Structure | Yield |
|---|---|---|
| XIIa | 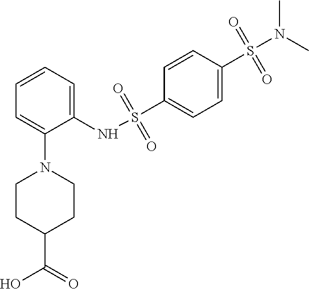 [SMILES: CN(C)S(=O)(=O)c1ccc(S(=O)(=O)Nc2ccccc2N2CCC(C(=O)O)CC2)cc1] | 99% |

General Procedures for the Synthesis of Final Compounds (Compounds of Formula I)

Method A.

To a cold (0° C.) solution of a suitable compound of formula II or formula III (1.0 eq) in DCM a commercially available sulphonyl chloride (1.2 eq) was added while maintaining the temperature at 0° C. Then, TEA (2.0 eq) or pyridine (2.0 eq) was added. The reaction was magnetically stirred at rt from 2 h to overnight. The reaction mixture was transferred into a separating funnel and was washed with water, a saturated solution of NaHCO₃ and brine. The organic layer was dried over anhydrous Na₂SO₄, filtered and evaporated under reduced pressure. The residue was purified by flash silica gel column chromatography as reported in the specific examples.

Method B.

To a cold (0° C.) solution of compound of formula VI (1.0 eq) in DCM a suitable commercially available amines (1.2 eq) and then TEA or pyridine (3.0 eq) were added dropwise. The reaction was stirred at rt on. Then the reaction was partitioned with DCM and water, the organic layer was washed with brine, dried over Na₂SO₄ anhydrous, filtered and evaporated under reduced pressure. The residue was purified by flash silica gel column chromatography as reported in the specific examples.

Method C.

To a cooled suspension of NaH (2.0 eq) in DMF (5 ml) a compound of formula VIII (1 eq) was added and the reaction mixture was stirred at rt for 30 min. Then the reaction mixture was cooled back to 0° C. and CH₃I or CH₃CH₂I or (CH₃)₂CHI (1.5 eq) was added. The reaction mixture was allowed to warm to rt and stirred from 2 h to overnight or heated to 100° C. and stirred overnight. Then it was cooled at 0° C., quenched with H₂O and extracted with EtOAc (×3). The combined organic layer was dried (Na₂SO₄), filtered and evaporated under reduced pressure. The residue was purified by flash silica gel column chromatography as reported in the specific examples.

Method D.

To a cold (0° C.) solution of compound of formula IX (1.0 eq) in DCM, a commercially available acyl chloride (1.1 eq.) and pyridine (1.5 eq.) were added dropwise. The reaction mixture was stirred at rt on. Then water was added, and the reaction mixture was extracted with DCM. The organic layer was washed with brine, dried over Na₂SO₄ anhydrous, filtered and evaporate under reduced pressure. The residue was purified by flash silica gel column chromatography as reported in the specific examples.

Method E.

A mixture of compound of formula V or formula XII (1.0 eq) and HATU (1.2 eq) in DCM was stirred at rt for 10 min. Then, the opportune commercially available amine (1.2 eq) and TEA (3.0 eq) were added, and the resulting mixture was stirred at rt on. The solvent was removed in vacuo. The residue was purified by flash silica gel column chromatography as reported in the specific examples.

Method F.

To a solution of compound of formula VII (1.0 eq) in DMF (10 ml) K₂CO₃ (1.5 eq) was added followed by CH₃I (3.0 eq). The reaction was magnetically stirred at rt on. Then the solvent was removed, and water was added. The resulting mixture was extracted with EtOAc. The combined organic layers were dried over Na₂SO₄ anhydrous, filtered and evaporated under reduced pressure. The residue was purified by flash silica gel column chromatography as reported in the specific examples.

### EXAMPLES

The invention is further illustrated by the following examples.

### Example 1

The title compound was prepared by the general method A, starting from commercially available amine CAS: 39643-31-7 and sulphonyl chloride CAS: 90001-64-2, using TEA as the base. It was purified by flash silica gel column chromatography using a linear gradient of ethyl acetate in hexane from 0% to 10%. (y=63%).

### Example 2

The title compound was prepared by the general method A, starting from commercially available amine CAS: 39643-31-7 and sulphonyl chloride CAS: 19715-49-2, using pyridine as the base. It was purified by flash silica gel column chromatography using a linear gradient of ethyl acetate in hexane from 0% to 10%. (y=89%).

### Example 3

The title compound was prepared by the general method A, starting from commercially available amine CAS: 2836-03-5 and sulphonyl chloride CAS: 677782-39-7, using TEA as the base. It was purified by flash silica gel column chromatography using a linear gradient of ethyl acetate in hexane from 0% to 50%. (y=23%).

### Example 4

The title compound was prepared by the general method A, starting from commercially available amine CAS: 1250695-37-4 and sulphonyl chloride (CAS: 677782-39-7), using TEA as the base. It was purified by flash silica gel column chromatography using a linear gradient of ethyl acetate in hexane from 0% to 20% (y=11%).

### Example 5

The title compound was prepared by the general method C, from intermediate VIIIa and iodomethane (CAS: 74-88-4) at rt for 2 h and purified by flash silica gel column chromatography using a linear gradient of ethyl acetate in hexane from 0% to 50% (y=60%).

### Example 6

The title compound was prepared by the general method A, starting from commercially available amine CAS 39643-31-7 and sulphonyl chloride CAS 677782-39-7, using TEA as the base. It was purified by flash silica gel column chromatography using a linear gradient of ethyl acetate in hexane from 0% to 30% (y=69%).

### Example 7

The title compound was prepared by the general method E, starting from commercially available carboxylic acid CAS: 91013-44-4 and amine CAS: 21627-58-7. It was purified by flash silica gel column chromatography using a linear gradient of ethyl acetate in hexane from 0% to 45%. (y=87%).

### Example 8

The title compound was prepared by the general method A, starting from commercially available amine CAS: 180629-70-3 and sulphonyl chloride CAS: 677782-39-7, using TEA as the base. It was purified by flash silica gel column chromatography using a linear gradient of ethyl acetate in hexane from 0% to 50%. (y=41%).

### Example 9

The title compound was prepared by the general method A, starting from commercially available amine CAS: 246247-91-6 and sulphonyl chloride CAS: 677782-39-7, using TEA as the base. It was purified by flash silica gel column chromatography using a linear gradient of ethyl acetate in hexane from 0% to 50%. (y=47%).

### Example 10

The title compound was prepared by the general method C, from intermediate VIIIb and iodomethane (CAS: 74-88-4) at rt for 2 h and purified by flash silica gel column chromatography using a linear gradient of ethyl acetate in hexane from 0% to 50% (y=75%).

### Example 11

The title compound was prepared by the general method A, starting from commercially available amine CAS: 39643-31-7 and sulphonyl chloride CAS: 103011-38-7, using TEA as the base. It was purified by flash silica gel column chromatography using a linear gradient of ethyl acetate in hexane from 0% to 20% (y=46%).

### Example 12

The title compound was prepared by the general method A, starting from commercially available amine CAS: 252758-95-5 and sulphonyl chloride CAS: 677782-39-7, using TEA as the base. It was purified by flash silica gel column chromatography using a linear gradient of ethyl acetate in hexane from 0% to 70% (y=60%).

### Example 13

The title compound was prepared by the general method A, starting from commercially available amine CAS: 170017-74-0 and sulphonyl chloride CAS: 677782-39-7, using pyridine as the base. It was purified by flash silica gel column chromatography using a linear gradient of ethyl acetate in hexane from 0% to 50% (y=96%).

### Example 14

The title compound was prepared by the general method E, starting from intermediate XIIa and commercially available amine CAS: 627-35-0. It was purified by flash silica gel column chromatography using a linear gradient of ethyl acetate in hexane from 0% to 40% (y=88%).

### Example 15

The title compound was prepared by the general method C, from intermediate VIIIe and iodomethane (CAS: 74-88-4) at rt for 2 h and purified by flash silica gel column chromatography using a linear gradient of ethyl acetate in hexane from 0% to 50% (y=54%).

### Example 16

The title compound was prepared by the general method A, starting from commercially available amine CAS: 1250695-37-4 and sulphonyl chloride CAS: 179400-17-0, using pyridine as the base. It was purified by flash silica gel column chromatography using a linear gradient of ethyl acetate in hexane from 0% to 10% (y=72%).

### Example 17

The title compound was prepared by the general method A, starting from commercially available amine CAS: 1250695-37-4 and sulphonyl chloride CAS: 56921-00-7, using pyridine as the base. It was purified by flash silica gel column chromatography using a linear gradient of ethyl acetate in hexane from 0% to 10% (y=62%).

### Example 18

The title compound was prepared by the general method A, starting from commercially available amine CAS: 170017-74-0 and sulphonyl chloride CAS: 16712-69-9, using pyridine as the base. It was purified by flash silica gel column chromatography using a linear gradient of ethyl acetate in hexane from 0% to 40% (y=95%).

### Example 19

The title compound was prepared by the general method A, starting from intermediate IId and sulphonyl chloride CAS: 16712-69-9, using pyridine as the base. It was purified by flash silica gel column chromatography using a linear gradient of ethyl acetate in hexane from 0% to 20% (y=87%).

### Example 20

The title compound was prepared by the general method C, from intermediate VIIIa and iodoethane (CAS: 75-03-6) at rt for 2 h and purified by flash silica gel column chromatography using a linear gradient of ethyl acetate in hexane from 0% to 50% (y=67%).

### Example 21

The title compound was prepared by the general method F, starting from Example 17. It was purified by flash silica gel column chromatography using a linear gradient of ethyl acetate in hexane from 0% to 50% (y=56%).

### Example 22

The title compound was prepared by the general method B, starting from intermediate VIa and commercially available amine CAS: 110-89-4. It was purified by flash silica gel column chromatography using a linear gradient of ethyl acetate in hexane from 0% to 70%. (y=87%).

### Example 23

The title compound was prepared by the general method A, starting from commercially available amine CAS: 39643-31-7 and sulphonyl chloride CAS: 1899-93-0, using pyridine as the base. It was purified by flash silica gel column chromatography using a linear gradient of ethyl acetate in hexane from 0% to 10% (y=75%).

### Example 24

The title compound was prepared by the general method A, starting from intermediate IIa and sulphonyl chloride CAS: 677782-39-7, using TEA as the base. It was purified by flash silica gel column chromatography using a linear gradient of ethyl acetate in hexane from 0% to 70% (y=9%).

### Example 25

The title compound was prepared by the general method A, starting from commercially available amine CAS: 1018636-14-0 and sulphonyl chloride CAS: 677782-39-7, using TEA as the base. It was purified by flash silica gel column chromatography using a linear gradient of ethyl acetate in hexane from 0% to 70% (y=13%).

### Example 26

The title compound was prepared by the general method A, starting from commercially available amine CAS: 1018624-48-0 and sulphonyl chloride CAS: 677782-39-7, using pyridine as the base. It was purified by flash silica gel column chromatography using a linear gradient of ethyl acetate in hexane from 0% to 80% (y=90%).

### Example 27

The title compound was prepared by the general method A, starting from commercially available amine CAS: 5585-33-1 and sulphonyl chloride CAS: 677782-39-7, using TEA as the base. It was purified by flash silica gel column chromatography using a linear gradient of ethyl acetate in hexane from 0% to 50% (y=39%).

### Example 28

The title compound was prepared by the general method A, starting from commercially available amine CAS: 170017-74-0 and sulphonyl chloride CAS: 54997-90-9, using pyridine as the base. It was purified by flash silica gel column chromatography using a linear gradient of ethyl acetate in hexane from 0% to 40% (y=87%).

### Example 29

The title compound was prepared by the general method A, starting from intermediate IId and sulphonyl chloride CAS: 54997-90-9, using pyridine as the base. It was purified by flash silica gel column chromatography using a linear gradient of ethyl acetate in hexane from 0% to 20% (y=96%).

### Example 30

The title compound was prepared by the general method A, starting from commercially available amine CAS: 19577-83-4 and sulphonyl chloride CAS: 677782-39-7, using pyridine as the base. It was purified by flash silica gel column chromatography using a linear gradient of ethyl acetate in hexane from 20% to 100% (y=96%).

### Example 31

The title compound was prepared by the general method A, starting from intermediate IId and sulphonyl chloride CAS: 98995-40-5, using pyridine as the base. It was purified by flash silica gel column chromatography using a linear gradient of ethyl acetate in hexane from 0% to 25% (y=66%).

### Example 32

The title compound was prepared by the general method A, starting from commercially available amine CAS: 21627-58-7 and sulphonyl chloride CAS: 677782-39-7, using TEA as the base. It was purified by flash silica gel column chromatography using a linear gradient of ethyl acetate in hexane from 0% to 40% (y=15%).

### Example 33

The title compound was prepared by the general method A, starting from commercially available amine CAS: 854044-39-6 and sulphonyl chloride CAS: 677782-39-7, using pyridine as the base. It was purified by flash silica gel column chromatography using a linear gradient of ethyl acetate in hexane from 0% to 40% (y=65%).

### Example 35

The title compound was prepared by the general method A, starting from intermediate IIb and sulphonyl chloride CAS: 677782-39-7, using pyridine as the base. It was purified by flash silica gel column chromatography using a linear gradient of ethyl acetate in hexane from 0% to 60% (y=90%).

### Example 36

The title compound was prepared by the general method A, starting from commercially available amine CAS: 1602318-01-3 and sulphonyl chloride CAS: 677782-39-7, using pyridine as the base. It was purified by flash silica gel column chromatography using a linear gradient of ethyl acetate in hexane from 0% to 40% (y=91%).

### Example 37

The title compound was prepared by the general method A, starting from commercially available amine CAS: 1272785-78-0 and sulphonyl chloride CAS: 677782-39-7, using pyridine as the base. It was purified by flash silica gel column chromatography using a linear gradient of ethyl acetate in hexane from 0% to 40% (y=74%).

### Example 38

The title compound was prepared by the general method A, starting from commercially available amine CAS: 252758-96-6 and sulphonyl chloride CAS: 677782-39-7, using pyridine as the base. It was purified by flash silica gel column chromatography using a linear gradient of ethyl acetate in hexane from 30% to 100% (y=98%).

### Example 39

The title compound was prepared by the general method A, starting from commercially available amine CAS: 39643-31-7 and sulphonyl chloride CAS: 98995-40-5, using pyridine as the base. It was purified by flash silica gel column chromatography using a linear gradient of ethyl acetate in hexane from 0% to 25% (y=60%).

### Example 40

The title compound was prepared by the general method A, starting from commercially available amine CAS: 1996133-97-1 and sulphonyl chloride CAS: 677782-39-7, using pyridine as the base. It was purified by flash silica gel column chromatography using a linear gradient of ethyl acetate in hexane from 0% to 50% (y=55%).

### Example 41 and Example 42

The title compounds, were obtained by enantiomeric separation of the racemate of Example 40; for separation procedures, see analytical methods.

### Example 43

The title compound was prepared by the general method A, starting from commercially available amine CAS: 955428-44-1 and sulphonyl chloride CAS: 677782-39-7, using pyridine as the base. It was purified by flash silica gel column chromatography using a linear gradient of methanol in dichloromethane from 0% to 5% (y=68%).

### Example 44

The title compound was prepared by the general method A, starting from commercially available amine CAS: 1554044-58-4 and sulphonyl chloride CAS: 677782-39-7, using pyridine as the base. It was purified by flash silica gel column chromatography using a linear gradient of ethyl acetate in hexane from 0% to 70% (y=57%).

### Example 45

The title compound was prepared by the general method A, starting from commercially available amine CAS: 1018624-51-5 and sulphonyl chloride CAS: 677782-39-7, using pyridine as the base. It was purified by flash silica gel column chromatography using a linear gradient of ethyl acetate in hexane from 0% to 70% (y=50%).

### Example 46

The title compound was prepared by the general method C, from intermediate VIIIa and 2-iodopropane (CAS: 75-30-9) at rt overnight and purified by flash silica gel chromatography using a linear gradient of ethyl acetate in hexane from 0% to 70% (y=22%).

### Example 47

The title compound was prepared by the general method A, starting from commercially available amine CAS: 170017-74-0 and sulphonyl chloride CAS: 133-59-5, using pyridine as the base. It was purified by flash silica gel column chromatography using a linear gradient of ethyl acetate in hexane from 0% to 70% (y=85%).

### Example 49

The title compound was prepared by the general method C, from intermediate VIIIf and iodomethane (CAS: 74-88-4) at rt for 2 h and purified by flash silica gel chromatography using a linear gradient of ethyl acetate in hexane from 0% to 50% (y=80%).

### Example 50

The title compound was prepared by the general method A, starting from commercially available amine CAS: 170017-74-0 and sulphonyl chloride CAS: 609-60-9, using pyridine as the base. It was purified by flash silica gel column chromatography using a linear gradient of ethyl acetate in hexane from 0% to 70% (y=94%).

### Example 51

The title compound was prepared by the general method A, starting from commercially available amine CAS: 170017-74-0 and sulphonyl chloride CAS: 2905-30-8, using pyridine as the base. It was purified by flash silica gel column chromatography using a linear gradient of ethyl acetate in hexane from 0% to 70% (y=86%).

### Example 52

The title compound was prepared by the general method D, starting from intermediate IXa and commercially available acyl chloride CAS: 79-22-1. It was purified by flash silica gel column chromatography using a linear gradient of ethyl acetate in hexane from 0% to 70% (y=33%).

### Example 53

The title compound was prepared by the general method D, starting from intermediate IXa and commercially available acyl chloride CAS: 27746-99-2. It was purified by flash silica gel column chromatography using a linear gradient of ethyl acetate in hexane from 0% to 40% (y=40%).

### Example 54

The title compound was prepared by the general method A, starting from commercially available amine CAS: 170017-74-0 and sulphonyl chloride CAS: 2905-27-3, using pyridine as the base. It was purified by flash silica gel column chromatography using a linear gradient of ethyl acetate in hexane from 0% to 70% (y=82%).

### Example 55

The title compound was prepared by the general method B, starting from intermediate VIa and commercially available amine CAS: 771-99-3. It was purified by flash silica gel column chromatography using a linear gradient of ethyl acetate in hexane from 0% to 40% (y=33%).

### Example 56

The title compound was prepared by the general method A, starting from commercially available amine CAS: 170017-74-0 and sulphonyl chloride CAS: 80466-80-4, using pyridine as the base. It was purified by flash silica gel column chromatography using a linear gradient of ethyl acetate in hexane from 0% to 60% (y=59%).

### Example 57

The title compound was prepared by the general method B, starting from intermediate VIa and commercially available amine CAS: 3202-33-3. It was purified by flash silica gel column chromatography using a linear gradient of ethyl acetate in hexane from 0% to 50% (y=49%).

### Example 58

The title compound was prepared by the general method A, starting from commercially available amine CAS: 170017-74-0 and sulphonyl chloride CAS: 166964-26-7, using pyridine as the base. It was purified by flash silica gel column chromatography using a linear gradient of ethyl acetate in hexane from 0% to 50% (y=95%).

### Example 59

The title compound was prepared by the general method A, starting from commercially available amine CAS: 170017-74-0 and sulphonyl chloride CAS: 97272-04-3, using pyridine as the base. It was purified by flash silica gel column chromatography using a linear gradient of ethyl acetate in hexane from 0% to 50% (y=96%).

### Example 60

The title compound was prepared by the general method A, starting from commercially available amine CAS: 170017-74-0 and sulphonyl chloride CAS: 166337-57-1, using pyridine as the base. It was purified by flash silica gel column chromatography using a linear gradient of ethyl acetate in hexane from 0% to 40% (y=50%).

### Example 61

The title compound was prepared by the general method B, starting from intermediate VIa and commercially available amine CAS: 110-91-8. It was purified by flash silica gel column chromatography using a linear gradient of ethyl acetate in hexane from 0% to 50% (y=65%).

### Example 62 and Example 63

The title compounds, were obtained by enantiomeric separation of the racemate of Example 57; for separation procedures, see analytical methods.

### Example 64

The title compound was prepared by the general method A, starting from commercially available amine CAS: 170017-74-0 and sulphonyl chloride CAS: 80466-79-1, using pyridine as the base. It was purified by flash silica gel column chromatography using a linear gradient of ethyl acetate in hexane from 0% to 35% (y=93%).

### Example 65

The title compound was prepared by the general method A, starting from intermediate IIc and sulphonyl chloride CAS: 609-60-9, using pyridine as the base. It was purified by flash silica gel column chromatography using a linear gradient of ethyl acetate in hexane from 0% to 40% (y=95%).

### Example 66

The title compound was prepared by the general method A, starting from commercially available amine CAS: 1018624-51-5 and sulphonyl chloride CAS: 609-60-9, using pyridine as the base. It was purified by flash silica gel column chromatography using a linear gradient of ethyl acetate in hexane from 0% to 35% (y=99%).

### Example 67

The title compound was prepared by the general method A, starting from commercially available amine CAS: 1018624-51-5 and sulphonyl chloride CAS: 166964-26-7, using pyridine as the base. It was purified by flash silica gel column chromatography using a linear gradient of ethyl acetate in hexane from 0% to 35% (y=98%).

### Example 68

The title compound was prepared by the general method A, starting from commercially available amine CAS: 1018624-51-5 and sulphonyl chloride CAS: 97272-04-3, using pyridine as the base. It was purified by flash silica gel column chromatography using a linear gradient of ethyl acetate in hexane from 0% to 35% (y=99%).

### Example 70

The title compound was prepared by the general method D, starting from intermediate IXa and commercially available acyl chloride CAS: 541-41-3. It was purified by flash silica gel column chromatography using a linear gradient of ethyl acetate in hexane from 0% to 60% (y=68%).

### Example 71

The title compound was prepared by the general method D, starting from intermediate IXa and commercially available acyl chloride CAS: 79-03-8. It was purified by flash silica gel column chromatography using a linear gradient of ethyl acetate in hexane from 0% to 100% (y=75%).

### Example 72

The title compound was prepared by the general method A, starting from commercially available amine CAS: 170017-74-0 and sulphonyl chloride CAS: 173669-61-9, using pyridine as the base. It was purified by flash silica gel column chromatography using a linear gradient of ethyl acetate in hexane from 0% to 50% (y=99%).

### Example 73

The title compound was prepared by the general method D, starting from intermediate IXa and commercially available acyl chloride CAS: 98-88-4. It was purified by flash silica gel column chromatography using a linear gradient of ethyl acetate in hexane from 0% to 70% (y=92%).

### Example 74

The title compound was prepared by the general method A, starting from commercially available amine CAS: 72752-54-6 and sulphonyl chloride CAS: 677782-39-7, using pyridine as the base. It was purified by flash silica gel column chromatography using a linear gradient of ethyl acetate in hexane from 0% to 50% (y=83%).

### Example 75

The title compound was prepared by the general method A, starting from intermediate IIc and sulphonyl chloride CAS: 677782-39-7, using pyridine as the base. It was purified by flash silica gel column chromatography using a linear gradient of ethyl acetate in hexane from 0% to 50% (y=98%).

### Example 76

The title compound was prepared by the general method A, starting from intermediate IId and sulphonyl chloride CAS: 677782-39-7, using pyridine as the base. It was purified by flash silica gel column chromatography using a linear gradient of ethyl acetate in hexane from 0% to 40% (y=80%).

### Example 77

The title compound was prepared by the general method A, starting from intermediate IIe and sulphonyl chloride CAS: 677782-39-7, using pyridine as the base. It was purified by flash silica gel column chromatography using a linear gradient of ethyl acetate in hexane from 0% to 50% (y=96%).

### Example 78

The title compound was prepared by the general method A, starting from commercially available amine CAS: 199105-03-8 and sulphonyl chloride CAS: 677782-39-7, using pyridine as the base. It was purified by flash silica gel column chromatography using a linear gradient of ethyl acetate in hexane from 0% to 80% (y=80%).

### Example 79

The title compound was prepared by the general method D, starting from intermediate IXa and commercially available acyl chloride CAS: 5271-67-0. It was purified by flash silica gel column chromatography using a linear gradient of ethyl acetate in hexane from 0% to 80% (y=66%).

### Example 80

The title compound was prepared by the general method D, starting from intermediate IXa and commercially available acyl chloride CAS: 54237-09-1. It was purified by flash silica gel column chromatography using a linear gradient of ethyl acetate in hexane from 20% to 100% (y=59%).

### Example 81

The title compound was prepared by the general method D, starting from intermediate IXa and commercially available acyl chloride CAS: 39098-97-0. (y=55%). It was purified by flash silica gel column chromatography using a linear gradient of ethyl acetate in hexane from 20% to 100% (y=55%)

### Example 82

The title compound was prepared by the general method D, starting from intermediate IXa and commercially available acyl chloride CAS: 51493-02-8. It was purified by flash silica gel column chromatography using a linear gradient of ethyl acetate in hexane from 20% to 100% (y=45%).

### Example 83

The title compound was prepared by the general method A, starting from commercially available amine CAS: 170017-74-0 and sulphonyl chloride CAS: 98-59-9, using pyridine as the base. It was purified by flash silica gel column chromatography using a linear gradient of ethyl acetate in hexane from 0% to 50% (y=55%).

### Example 84

The title compound was prepared by the general method A, starting from commercially available amine CAS: 170017-74-0 and sulphonyl chloride CAS: 98-68-0, using pyridine as the base. It was purified by flash silica gel column chromatography using a linear gradient of ethyl acetate in hexane from 0% to 50% (y=61%).

### Example 85

The title compound was prepared by the general method A, starting from commercially available amine CAS: 51627-46-4 and sulphonyl chloride CAS: 677782-39-7, using pyridine as the base. It was purified by flash silica gel column chromatography using a linear gradient of ethyl acetate in hexane from 0% to 20% (y=40%).

### Example 86

The title compound was prepared by the general method A, starting from intermediate IIf and sulphonyl chloride CAS: 677782-39-7, using pyridine as the base. It was purified by flash silica gel column chromatography using a linear gradient of ethyl acetate in hexane from 20% to 100% (y=44%).

### Example 87

The title compound was prepared by the general method E, starting from intermediate XIIa and commercially available amine CAS: 107-10-8. It was purified by flash silica gel column chromatography using a linear gradient of ethyl acetate in hexane from 20% to 100% (y=98%).

### Example 88

The title compound was prepared by the general method E, starting from intermediate XIIa and commercially available amine CAS: 78-81-9. It was purified by flash silica gel column chromatography using a linear gradient of ethyl acetate in hexane from 20% to 100% (y=98%).

### Example 89

The title compound was prepared by the general method A, starting from intermediate IIg and sulphonyl chloride CAS: 677782-39-7, using pyridine as the base. It was purified by flash silica gel column chromatography using a linear gradient of ethyl acetate in hexane from 20% to 100% (y=97%).

### Example 90

The title compound was prepared by the general method A, starting from intermediate IId and sulphonyl chloride CAS: 98-59-9, using pyridine as the base. It was purified by flash silica gel column chromatography using a linear gradient of ethyl acetate in hexane from 0% to 70% (y=98%).

### Example 91

The title compound was prepared by the general method A, starting from intermediate IId and sulphonyl chloride CAS: 98-68-0, using pyridine as the base. It was purified by flash silica gel column chromatography using a linear gradient of ethyl acetate in hexane from 0% to 70% (y=98%).

### Example 92

The title compound was prepared by the general method A, starting from commercially available amine CAS: 170017-74-0 and sulphonyl chloride CAS: 369638-66-4, using pyridine as the base. It was purified by flash silica gel column chromatography using a linear gradient of ethyl acetate in hexane from 0% to 50% (y=94%).

### Example 93 and Example 94

The title compounds, were obtained by enantiomeric separation of the racemate of Example 92; for separation procedures, see analytical methods.

### Example 95

The title compound was prepared by the general method A, starting from commercially available amine CAS: 170017-74-0 and sulphonyl chloride CAS: 120164-05-8, using pyridine as the base. It was purified by flash silica gel column chromatography using a linear gradient of ethyl acetate in hexane from 0% to 50% (y=90%).

### Example 96

The title compound was prepared by the general method C, from intermediate VIIIc and 2-iodopropane (CAS: 75-30-9) at 100° C. overnight and purified by flash silica gel chromatography using a linear gradient of ethyl acetate in hexane from 0% to 70% (y=13%).

### Example 97

The title compound was prepared by the general method A, starting from intermediate IIc and sulphonyl chloride CAS: 97272-04-3, using pyridine as the base. It was purified by flash silica gel column chromatography using a linear gradient of ethyl acetate in hexane from 0% to 40% (y=95%).

### Example 98

The title compound was prepared by the general method A, starting from intermediate IIc and sulphonyl chloride CAS: 478264-00-5, using pyridine as the base. It was purified by flash silica gel column chromatography using a linear gradient of ethyl acetate in hexane from 0% to 50% (y=70%).

### Example 99

The title compound was prepared by the general method A, starting from intermediate IIc and sulphonyl chloride CAS: 955085-16-2, using pyridine as the base. It was purified by flash silica gel column chromatography using a linear gradient of ethyl acetate in hexane from 0% to 50% (y=78%).

### Example 100

The title compound was prepared by the general method A, starting from intermediate IIh and sulphonyl chloride CAS: 677782-39-7, using pyridine as the base. It was purified by flash silica gel column chromatography using a linear gradient of ethyl acetate in hexane from 10% to 50% (y=32%).

### Example 101

The title compound was prepared by the general method A, starting from intermediate IIi and sulphonyl chloride CAS: 677782-39-7, using pyridine as the base. It was purified by flash silica gel column chromatography using a linear gradient of ethyl acetate in hexane from 10% to 50% (y=34%).

### Example 103

The title compound was prepared by the general method C, from intermediate VIIId and 2-iodopropane (CAS: 75-30-9) at 100° C. overnight and purified by flash silica gel chromatography using a linear gradient of ethyl acetate in hexane from 0% to 50% (y=53%).

### Example 105

The title compound was prepared by the general method A, starting from intermediate IIj and sulphonyl chloride CAS: 677782-39-7, using pyridine as the base. It was purified by flash silica gel column chromatography using a linear gradient of ethyl acetate in hexane from 0% to 70% (y=92%).

### Example 106

The title compound was prepared by the general method A, starting from intermediate IIk and sulphonyl chloride CAS: 677782-39-7, using pyridine as the base. It was purified by flash silica gel column chromatography using a linear gradient of ethyl acetate in hexane from 0% to 70% (y=76%).

Table 2 lists final compounds that were prepared according to the experimental procedures described before.

TABLE 2

| Example | Structure |
|---|---|
| 1 | 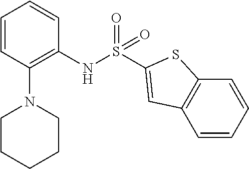 [SMILES: O=S(=O)(Nc1ccccc1N1CCCCC1)c1cc2ccccc2s1] |
| 2 | 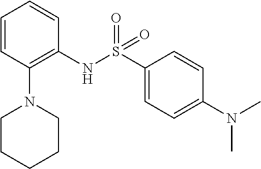 [SMILES: CN(C)c1ccc(S(=O)(=O)Nc2ccccc2N2CCCCC2)cc1] |
| 3 | 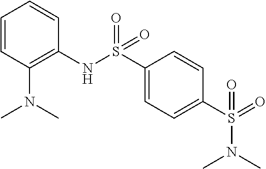 [SMILES: CN(C)c1ccccc1NS(=O)(=O)c1ccc(S(=O)(=O)N(C)C)cc1] |
| 4 | 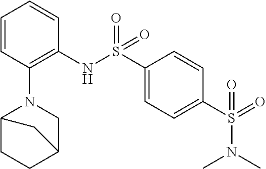 [SMILES: CN(C)S(=O)(=O)c1ccc(S(=O)(=O)Nc2ccccc2N2CC3CCC2C3)cc1] |
| 5 | 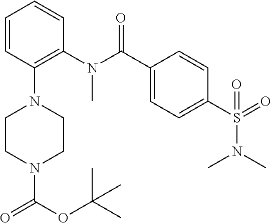 [SMILES: CN(C(=O)c1ccc(S(=O)(=O)N(C)C)cc1)c1ccccc1N1CCN(C(=O)OC(C)(C)C)CC1] |
| 6 | 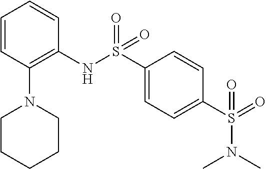 [SMILES: CN(C)S(=O)(=O)c1ccc(S(=O)(=O)Nc2ccccc2N2CCCCC2)cc1] |
| 7 | 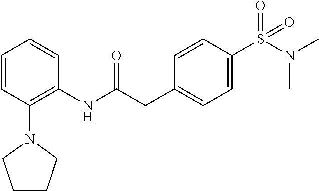 [SMILES: CN(C)S(=O)(=O)c1ccc(CC(=O)Nc2ccccc2N2CCCC2)cc1] |
| 8 | 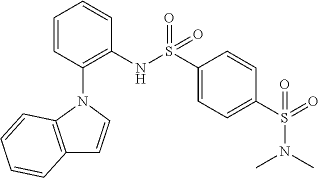 [SMILES: CN(C)S(=O)(=O)c1ccc(S(=O)(=O)Nc2ccccc2-n2ccc3ccccc32)cc1] |
| 9 | 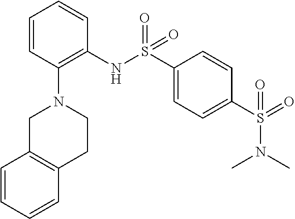 [SMILES: CN(C)S(=O)(=O)c1ccc(S(=O)(=O)Nc2ccccc2N2CCc3ccccc3C2)cc1] |
| 10 | 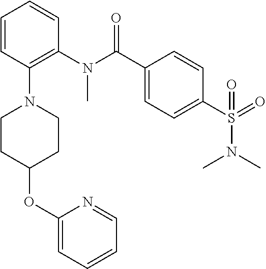 [SMILES: CN(C(=O)c1ccc(S(=O)(=O)N(C)C)cc1)c1ccccc1N1CCC(Oc2ccccn2)CC1] |
| 11 | 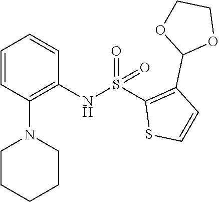 [SMILES: O=S(=O)(Nc1ccccc1N1CCCCC1)c1sccc1C1OCCO1] |
| 12 | 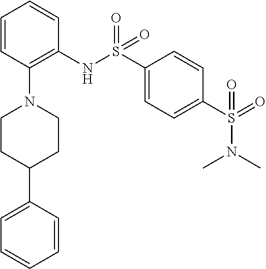 [SMILES: CN(C)S(=O)(=O)c1ccc(S(=O)(=O)Nc2ccccc2N2CCC(c3ccccc3)CC2)cc1] |
| 13 | 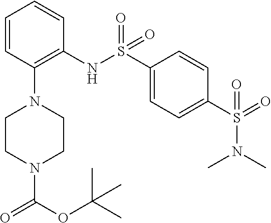 [SMILES: CN(C)S(=O)(=O)c1ccc(S(=O)(=O)Nc2ccccc2N2CCN(C(=O)OC(C)(C)C)CC2)cc1] |
| 14 | 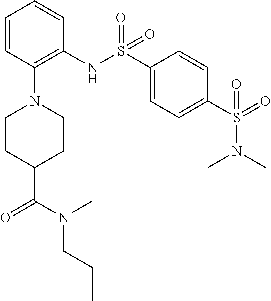 [SMILES: CCCN(C)C(=O)C1CCN(c2ccccc2NS(=O)(=O)c2ccc(S(=O)(=O)N(C)C)cc2)CC1] |
| 15 | 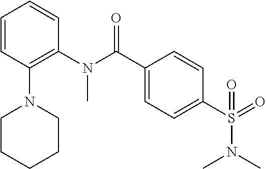 [SMILES: CN(C(=O)c1ccc(S(=O)(=O)N(C)C)cc1)c1ccccc1N1CCCCC1] |
| 16 | 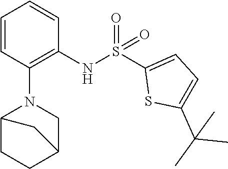 [SMILES: CC(C)(C)c1ccc(S(=O)(=O)Nc2ccccc2N2CC3CCC2C3)s1] |
| 17 | 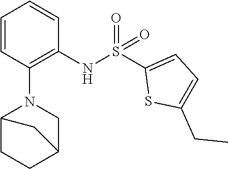 [SMILES: CCc1ccc(S(=O)(=O)Nc2ccccc2N2CC3CCC2C3)s1] |
| 18 | 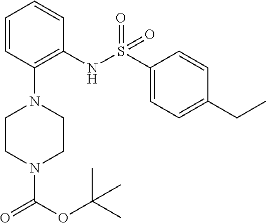 [SMILES: CCc1ccc(S(=O)(=O)Nc2ccccc2N2CCN(C(=O)OC(C)(C)C)CC2)cc1] |
| 19 | 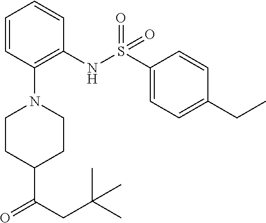 [SMILES: CCc1ccc(S(=O)(=O)Nc2ccccc2N2CCC(C(=O)CC(C)(C)C)CC2)cc1] |
| 20 | 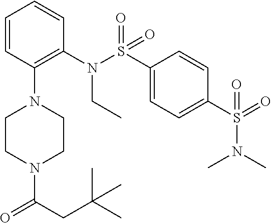 [SMILES: CCN(c1ccccc1N1CCN(C(=O)CC(C)(C)C)CC1)S(=O)(=O)c1ccc(S(=O)(=O)N(C)C)cc1] |
| 21 | 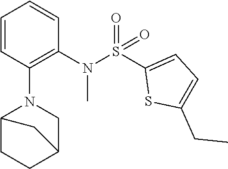 [SMILES: CCc1ccc(S(=O)(=O)N(C)c2ccccc2N2CC3CCC2C3)s1] |
| 22 | 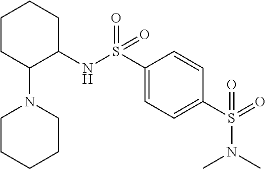 [SMILES: CN(C)S(=O)(=O)c1ccc(S(=O)(=O)NC2CCCCC2N2CCCCC2)cc1] |
| 23 | 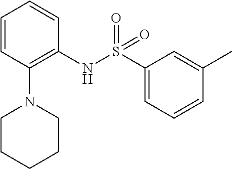 [SMILES: Cc1cccc(S(=O)(=O)Nc2ccccc2N2CCCCC2)c1] |
| 24 | 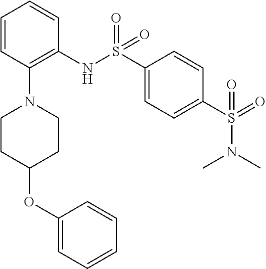 [SMILES: CN(C)S(=O)(=O)c1ccc(S(=O)(=O)Nc2ccccc2N2CCC(Oc3ccccc3)CC2)cc1] |
| 25 | 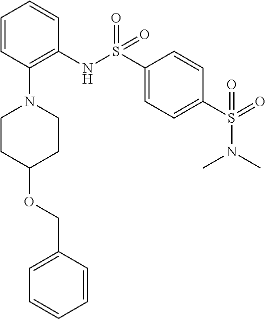 [SMILES: CN(C)S(=O)(=O)c1ccc(S(=O)(=O)Nc2ccccc2N2CCC(OCc3ccccc3)CC2)cc1] |
| 26 | 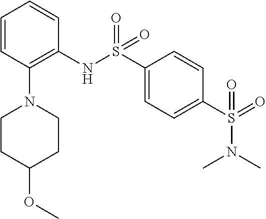 [SMILES: COC1CCN(c2ccccc2NS(=O)(=O)c2ccc(S(=O)(=O)N(C)C)cc2)CC1] |
| 27 | 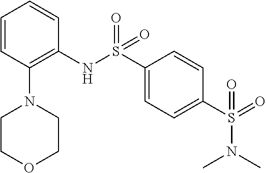 [SMILES: CN(C)S(=O)(=O)c1ccc(S(=O)(=O)Nc2ccccc2N2CCOCC2)cc1] |
| 28 | 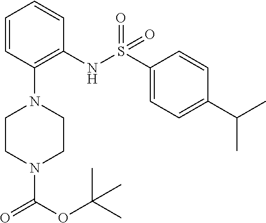 [SMILES: CC(C)c1ccc(S(=O)(=O)Nc2ccccc2N2CCN(C(=O)OC(C)(C)C)CC2)cc1] |
| 29 | 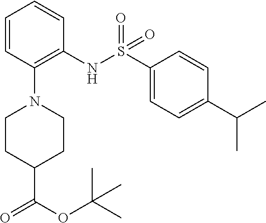 [SMILES: CC(C)c1ccc(S(=O)(=O)Nc2ccccc2N2CCC(C(=O)OC(C)(C)C)CC2)cc1] |
| 30 | 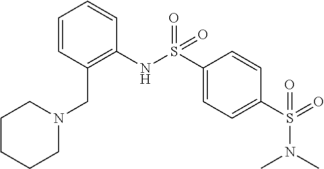 [SMILES: CN(C)S(=O)(=O)c1ccc(S(=O)(=O)Nc2ccccc2CN2CCCCC2)cc1] |
| 31 | 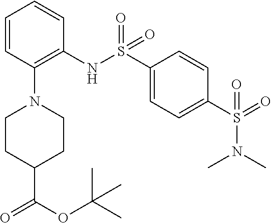 [SMILES: CN(C)S(=O)(=O)c1ccc(S(=O)(=O)Nc2ccccc2N2CCC(C(=O)OC(C)(C)C)CC2)cc1] |
| 32 | 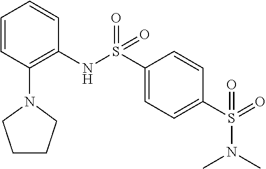 [SMILES: CN(C)S(=O)(=O)c1ccc(S(=O)(=O)Nc2ccccc2N2CCCC2)cc1] |
| 33 | 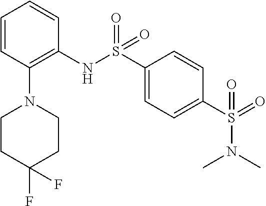 [SMILES: CN(C)S(=O)(=O)c1ccc(S(=O)(=O)Nc2ccccc2N2CCC(F)(F)CC2)cc1] |
| 35 | 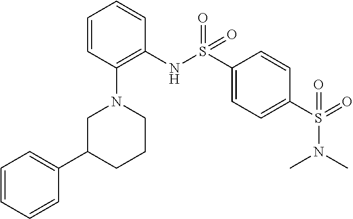 [SMILES: CN(C)S(=O)(=O)c1ccc(S(=O)(=O)Nc2ccccc2N2CCCC(c3ccccc3)C2)cc1] |
| 36 | 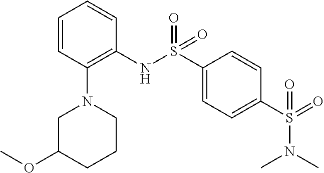 |
| 37 | 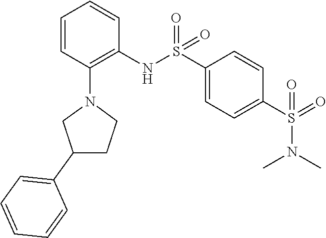 [SMILES: CN(C)S(=O)(=O)c1ccc(S(=O)(=O)Nc2ccccc2N2CCC(c3ccccc3)C2)cc1] |
| 38 | 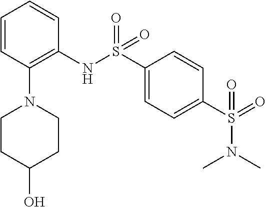 [SMILES: CN(C)S(=O)(=O)c1ccc(S(=O)(=O)Nc2ccccc2N2CCC(O)CC2)cc1] |
| 39 | 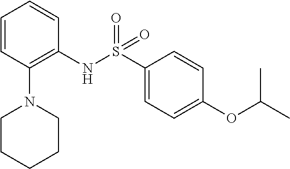 [SMILES: CC(C)Oc1ccc(S(=O)(=O)Nc2ccccc2N2CCCCC2)cc1] |
| 40 | 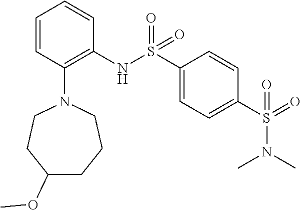 |
| 41 | 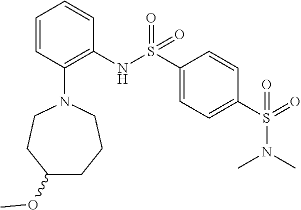 |
| 42 | 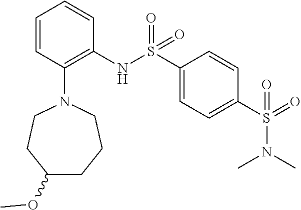 |
| 43 | 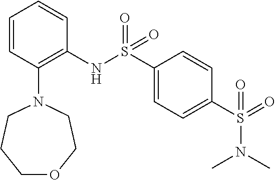 [SMILES: CN(C)S(=O)(=O)c1ccc(S(=O)(=O)Nc2ccccc2N2CCCOCC2)cc1] |
| 44 | 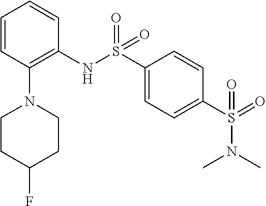 [SMILES: CN(C)S(=O)(=O)c1ccc(S(=O)(=O)Nc2ccccc2N2CCC(F)CC2)cc1] |
| 45 | 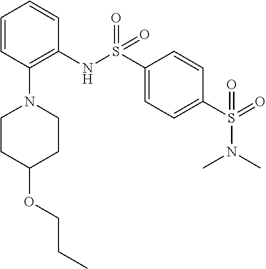 [SMILES: CCCOC1CCN(c2ccccc2NS(=O)(=O)c2ccc(S(=O)(=O)N(C)C)cc2)CC1] |
| 46 | 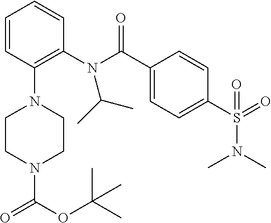 [SMILES: CC(C)N(C(=O)c1ccc(S(=O)(=O)N(C)C)cc1)c1ccccc1N1CCN(C(=O)OC(C)(C)C)CC1] |
| 47 | 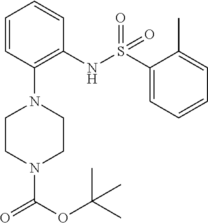 [SMILES: Cc1ccccc1S(=O)(=O)Nc1ccccc1N1CCN(C(=O)OC(C)(C)C)CC1] |
| 49 | 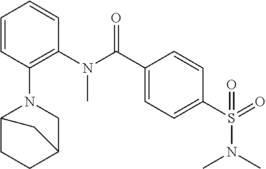 [SMILES: CN(C(=O)c1ccc(S(=O)(=O)N(C)C)cc1)c1ccccc1N1CC2CCC1C2] |
| 50 | 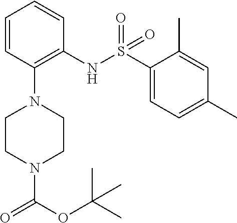 [SMILES: Cc1ccc(S(=O)(=O)Nc2ccccc2N2CCN(C(=O)OC(C)(C)C)CC2)c(C)c1] |
| 51 | 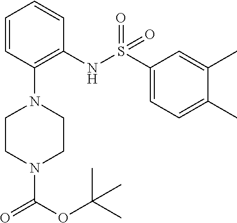 [SMILES: Cc1ccc(S(=O)(=O)Nc2ccccc2N2CCN(C(=O)OC(C)(C)C)CC2)cc1C] |
| 52 | 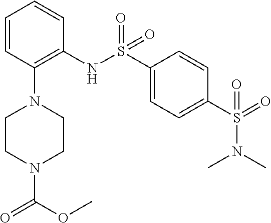 [SMILES: COC(=O)N1CCN(c2ccccc2NS(=O)(=O)c2ccc(S(=O)(=O)N(C)C)cc2)CC1] |
| 53 | 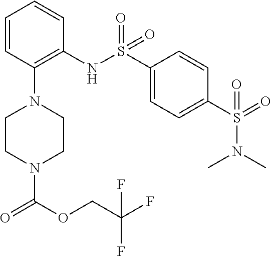 [SMILES: CN(C)S(=O)(=O)c1ccc(S(=O)(=O)Nc2ccccc2N2CCN(C(=O)OCC(F)(F)F)CC2)cc1] |
| 54 | 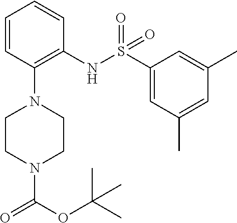 [SMILES: Cc1cc(C)cc(S(=O)(=O)Nc2ccccc2N2CCN(C(=O)OC(C)(C)C)CC2)c1] |
| 55 | 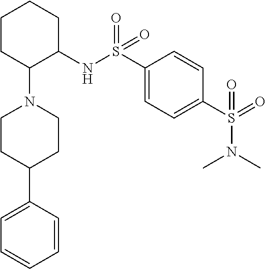 [SMILES: CN(C)S(=O)(=O)c1ccc(S(=O)(=O)NC2CCCCC2N2CCC(c3ccccc3)CC2)cc1] |
| 56 | 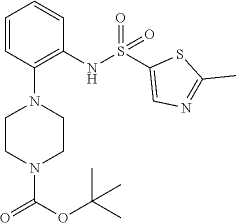 [SMILES: Cc1ncc(S(=O)(=O)Nc2ccccc2N2CCN(C(=O)OC(C)(C)C)CC2)s1] |
| 57 | 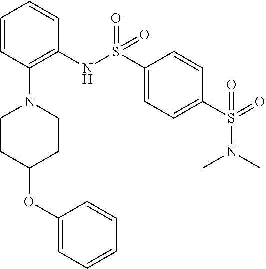 [SMILES: CN(C)S(=O)(=O)c1ccc(S(=O)(=O)Nc2ccccc2N2CCC(Oc3ccccc3)CC2)cc1] |
| 58 | 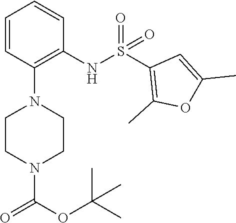 [SMILES: Cc1cc(S(=O)(=O)Nc2ccccc2N2CCN(C(=O)OC(C)(C)C)CC2)c(C)o1] |
| 59 | 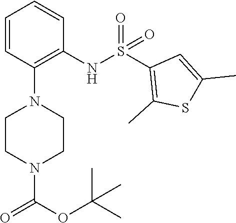 [SMILES: Cc1cc(S(=O)(=O)Nc2ccccc2N2CCN(C(=O)OC(C)(C)C)CC2)c(C)s1] |
| 60 | 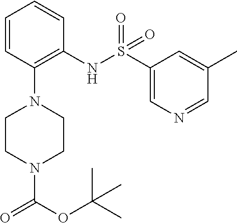 [SMILES: Cc1cncc(S(=O)(=O)Nc2ccccc2N2CCN(C(=O)OC(C)(C)C)CC2)c1] |
| 61 | 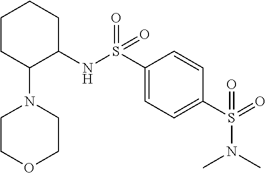 [SMILES: CN(C)S(=O)(=O)c1ccc(S(=O)(=O)NC2CCCCC2N2CCOCC2)cc1] |
| 62 | 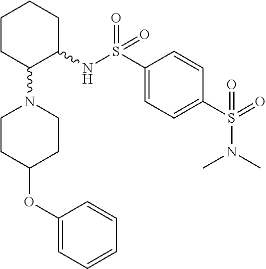 [SMILES: CN(C)S(=O)(=O)c1ccc(S(=O)(=O)NC2CCCCC2N2CCC(Oc3ccccc3)CC2)cc1] |
| 63 | 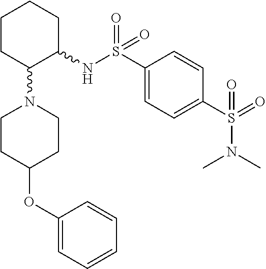 [SMILES: CN(C)S(=O)(=O)c1ccc(S(=O)(=O)NC2CCCCC2N2CCC(Oc3ccccc3)CC2)cc1] |
| 64 | 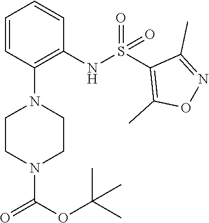 [SMILES: Cc1noc(C)c1S(=O)(=O)Nc1ccccc1N1CCN(C(=O)OC(C)(C)C)CC1] |
| 65 | 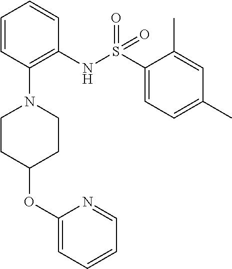 [SMILES: Cc1ccc(S(=O)(=O)Nc2ccccc2N2CCC(Oc3ccccn3)CC2)c(C)c1] |
| 66 | 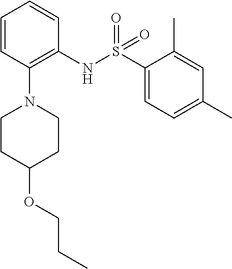 [SMILES: CCCOC1CCN(c2ccccc2NS(=O)(=O)c2ccc(C)cc2C)CC1] |
| 67 | 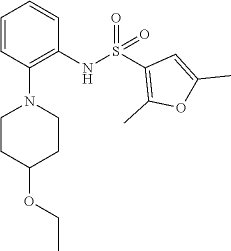 [SMILES: CCOC1CCN(c2ccccc2NS(=O)(=O)c2cc(C)oc2C)CC1] |
| 68 | 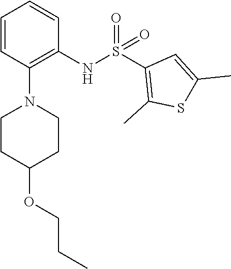 [SMILES: CCCOC1CCN(c2ccccc2NS(=O)(=O)c2cc(C)sc2C)CC1] |
| 70 | 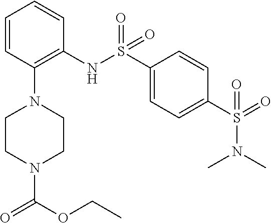 [SMILES: CCOC(=O)N1CCN(c2ccccc2NS(=O)(=O)c2ccc(S(=O)(=O)N(C)C)cc2)CC1] |
| 71 | 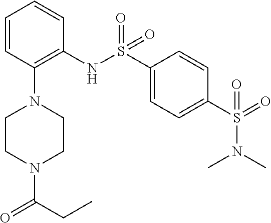 [SMILES: CCC(=O)N1CCN(c2ccccc2NS(=O)(=O)c2ccc(S(=O)(=O)N(C)C)cc2)CC1] |
| 72 | 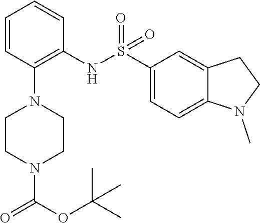 [SMILES: CN1CCc2cc(S(=O)(=O)Nc3ccccc3N3CCN(C(=O)OC(C)(C)C)CC3)ccc21] |
| 73 | 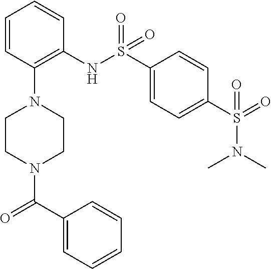 [SMILES: CN(C)S(=O)(=O)c1ccc(S(=O)(=O)Nc2ccccc2N2CCN(C(=O)c3ccccc3)CC2)cc1] |
| 74 | 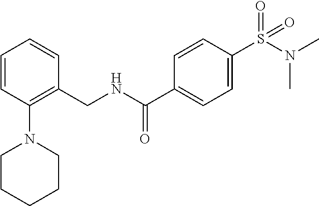 [SMILES: CN(C)S(=O)(=O)c1ccc(C(=O)NCc2ccccc2N2CCCCC2)cc1] |
| 75 | 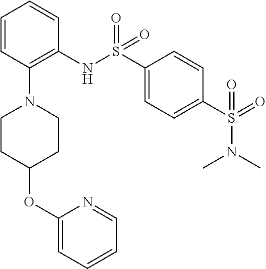 [SMILES: CN(C)S(=O)(=O)c1ccc(S(=O)(=O)Nc2ccccc2N2CCC(Oc3ccccn3)CC2)cc1] |
| 76 | 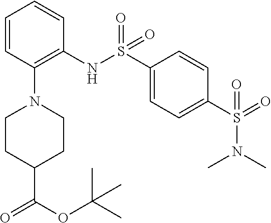 [SMILES: CN(C)S(=O)(=O)c1ccc(S(=O)(=O)Nc2ccccc2N2CCC(C(=O)OC(C)(C)C)CC2)cc1] |
| 77 | 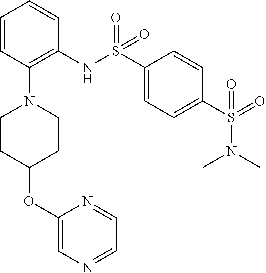 [SMILES: CN(C)S(=O)(=O)c1ccc(S(=O)(=O)Nc2ccccc2N2CCC(Oc3cnccn3)CC2)cc1] |
| 78 | 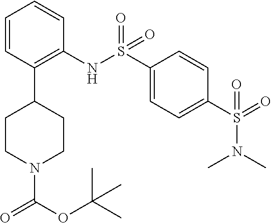 [SMILES: CN(C)S(=O)(=O)c1ccc(S(=O)(=O)Nc2ccccc2C2CCN(C(=O)OC(C)(C)C)CC2)cc1] |
| 79 | 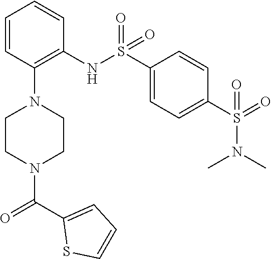 [SMILES: CN(C)S(=O)(=O)c1ccc(S(=O)(=O)Nc2ccccc2N2CCN(C(=O)c3cccs3)CC2)cc1] |
| 80 | 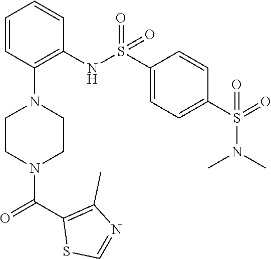 [SMILES: Cc1ncsc1C(=O)N1CCN(c2ccccc2NS(=O)(=O)c2ccc(S(=O)(=O)N(C)C)cc2)CC1] |
| 81 | 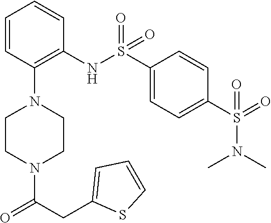 [SMILES: CN(C)S(=O)(=O)c1ccc(S(=O)(=O)Nc2ccccc2N2CCN(C(=O)Cc3cccs3)CC2)cc1] |
| 82 | 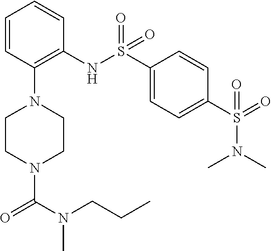 [SMILES: CCCN(C)C(=O)N1CCN(c2ccccc2NS(=O)(=O)c2ccc(S(=O)(=O)N(C)C)cc2)CC1] |
| 83 | 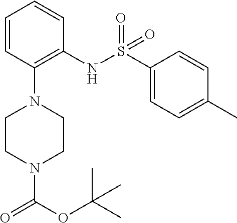 [SMILES: Cc1ccc(S(=O)(=O)Nc2ccccc2N2CCN(C(=O)OC(C)(C)C)CC2)cc1] |
| 84 | 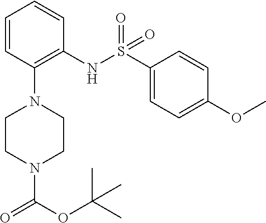 [SMILES: COc1ccc(S(=O)(=O)Nc2ccccc2N2CCN(C(=O)OC(C)(C)C)CC2)cc1] |
| 85 | 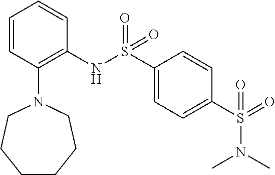 [SMILES: CN(C)S(=O)(=O)c1ccc(S(=O)(=O)Nc2ccccc2N2CCCCCC2)cc1] |
| 86 | 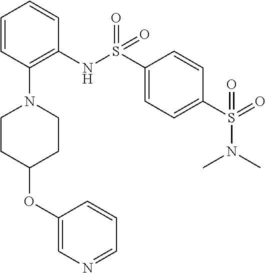 [SMILES: CN(C)S(=O)(=O)c1ccc(S(=O)(=O)Nc2ccccc2N2CCC(Oc3cccnc3)CC2)cc1] |
| 87 | 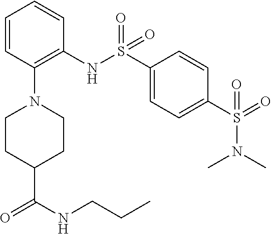 [SMILES: CCCNC(=O)C1CCN(c2ccccc2NS(=O)(=O)c2ccc(S(=O)(=O)N(C)C)cc2)CC1] |
| 88 | 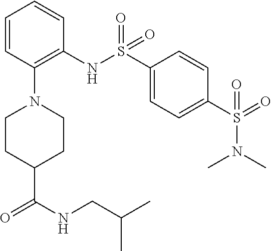 [SMILES: CC(C)CNC(=O)C1CCN(c2ccccc2NS(=O)(=O)c2ccc(S(=O)(=O)N(C)C)cc2)CC1] |
| 89 | 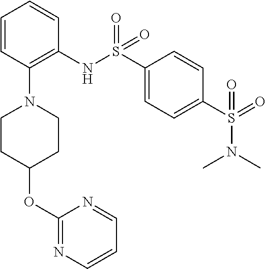 [SMILES: CN(C)S(=O)(=O)c1ccc(S(=O)(=O)Nc2ccccc2N2CCC(Oc3ncccn3)CC2)cc1] |
| 90 | 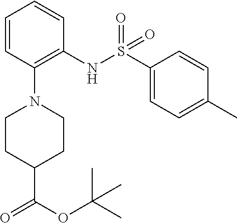 [SMILES: Cc1ccc(S(=O)(=O)Nc2ccccc2N2CCC(C(=O)OC(C)(C)C)CC2)cc1] |
| 91 | 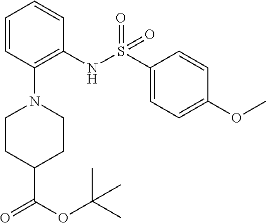 [SMILES: COc1ccc(S(=O)(=O)Nc2ccccc2N2CCC(C(=O)OC(C)(C)C)CC2)cc1] |
| 92 | 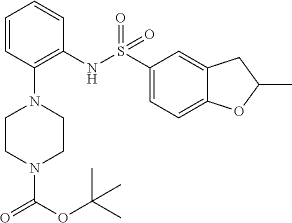 [SMILES: CC1Cc2cc(S(=O)(=O)Nc3ccccc3N3CCN(C(=O)OC(C)(C)C)CC3)ccc2O1] |
| 93 | 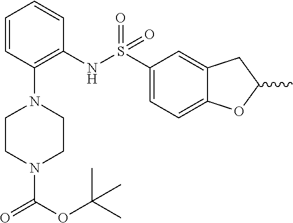 [SMILES: CC1Cc2cc(S(=O)(=O)Nc3ccccc3N3CCN(C(=O)OC(C)(C)C)CC3)ccc2O1] |
| 94 | 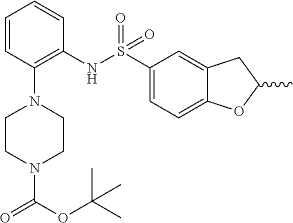 [SMILES: CC1Cc2cc(S(=O)(=O)Nc3ccccc3N3CCN(C(=O)OC(C)(C)C)CC3)ccc2O1] |
| 95 | 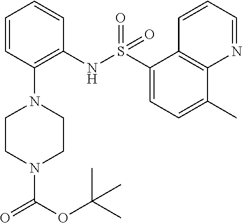 [SMILES: Cc1ccc(S(=O)(=O)Nc2ccccc2N2CCN(C(=O)OC(C)(C)C)CC2)c2cccnc12] |
| 96 | 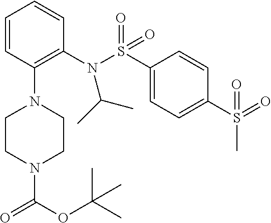 [SMILES: CC(C)N(c1ccccc1N1CCN(C(=O)OC(C)(C)C)CC1)S(=O)(=O)c1ccc(S(C)(=O)=O)cc1] |
| 97 | 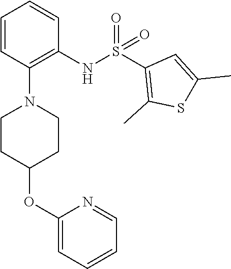 [SMILES: Cc1cc(S(=O)(=O)Nc2ccccc2N2CCC(Oc3ccccn3)CC2)c(C)s1] |
| 98 | 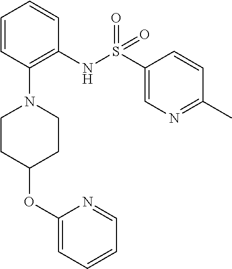 [SMILES: Cc1ccc(S(=O)(=O)Nc2ccccc2N2CCC(Oc3ccccn3)CC2)cn1] |
| 99 | 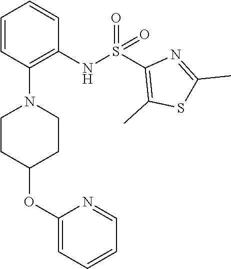 [SMILES: Cc1nc(S(=O)(=O)Nc2ccccc2N2CCC(Oc3ccccn3)CC2)c(C)s1] |
| 100 | 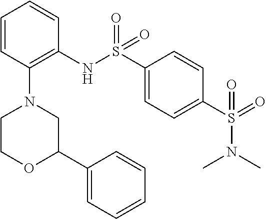 [SMILES: CN(C)S(=O)(=O)c1ccc(S(=O)(=O)Nc2ccccc2N2CCOC(c3ccccc3)C2)cc1] |
| 101 | 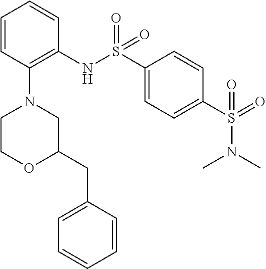 [SMILES: CN(C)S(=O)(=O)c1ccc(S(=O)(=O)Nc2ccccc2N2CCOC(Cc3ccccc3)C2)cc1] |
| 103 | 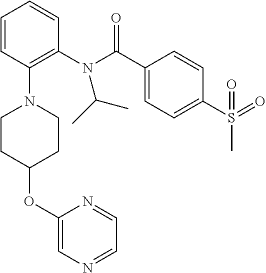 [SMILES: CC(C)N(C(=O)c1ccc(S(C)(=O)=O)cc1)c1ccccc1N1CCC(Oc2cnccn2)CC1] |
| 105 | 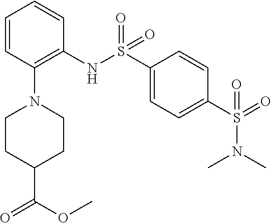 [SMILES: COC(=O)C1CCN(c2ccccc2NS(=O)(=O)c2ccc(S(=O)(=O)N(C)C)cc2)CC1] |
| 106 | 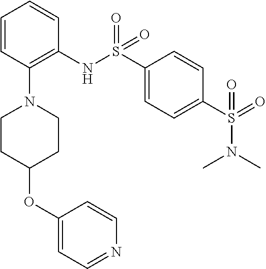 [SMILES: CN(C)S(=O)(=O)c1ccc(S(=O)(=O)Nc2ccccc2N2CCC(Oc3ccncc3)CC2)cc1] |

Analytical Procedures and Data

Purification System

Flash Chromatography (FC)

FC separations were performed on Biotage Isolera F our equipped with UV detector. Type of silica columns: Claricep Screw-on, Irregular, 40-60 μm, 12-80 g.

System Purification

Preparative HPLC

HPLC system WATERS Quaternary Gradient Mobile 2535 equipped with WATERS UVNisible Detector 2489 set to dual-wavelength UV detection. Two mobile phases were used, mobile phase A: water (MilliQ) 0,05% TFA; mobile phase B: acetonitrile (Chromasolv Sigma-Aldrich) 0,05% TFA, and the run gradient conditions were set specifically for each compound. The purifications were achieved on a LUNA 5 μm C18 150×21.2 Colum. An injection volume between 100 and 500 μl was applied, and the flow was 15 ml/min.

Racemate Separation

The two enantiomers Example 62 and 63 were obtained by resolution of the racemic mixture (Example 57) using a WATERS Quaternary Gradient Mobile 2535 equipped with WATERS UVNisible Detector 2489 set to a dual-wavelength UV detection at 245 and 275 nm. The chiral resolution was achieved on the Lux Amylose-1 column (250 mm×21.6 mm) using Hexane-Isopropanol 68:32 (v/v) as isocratic mobile phase; The sample was eluted from the column at a flow rate of 1.0 ml/min at room temperature (Pressure: ≈800 psi). The racemic mixture was dissolved in Ethanol and heated, at concentration of 1.0% (w/v) and the injection volume was 100 μL.

LCMS

LCMS—Procedure 1

The HPLC measurement was performed using a Dionex 3000 module comprising a quaternary pump with degasser, an autosampler, a column oven (set at 29° C.), a diode-array detector DAD and a column as specified in the respective methods below. Flow from the column was split to a MS spectrometer. The MS detector (LCQ Fleet Thermo Scientific) was configured with an electrospray ionization source. Mass spectra were acquired by scanning from 50 to 800 in 0.48 second. The capillary needle voltage was 5 kV in positive and negative ionization mode and the source temperature was maintained at 275° C. Nitrogen was used as the nebulizer gas, the flow was 8 l/min. Data acquisition was performed with Thermo Xcalibur Qual Browser. Reversed phase HPLC was carried out on a Kinetex XB-C18 column Phenomenex (1.7 μm, 50×2.1 mm) with a flow rate of 0.300 ml/min. Two mobile phases were used, mobile phase A: ammonium formate buffer solution at pH 3.5; mobile phase B: acetonitrile (Chromasolv Sigma-Aldrich), and they were employed to run a gradient conditions from 15% B for 0.5 minutes, from 15% to 98% in 4.0 minutes, 98% B for 1.35 minutes and 15% B in 0.10 minutes and hold these conditions for 2.75 minutes in order to re-equilibrate the column (Total Run Time 8.7 minutes). An injection volume of 1 μl was used.

LCMS—Procedure 2

The HPLC measurement was performed using a Vanquish module comprising a quaternary pump with degasser, an autosampler, a column oven (set at 29° C.), a diode-array detector DAD and a column as specified in the respective methods below. Flow from the column was split to a MS spectrometer. The MS detector (ISQEC) was configured with an electrospray ionization source. Mass spectra were acquired by scanning from 50 to 600 in 0.2 second. The capillary needle voltage was 3 kV in positive and 2 kV in negative ionization mode and the source temperature was maintained at 250° C. Nitrogen was used as the nebulizer gas. Data acquisition was performed with Chromeleon 7. Reversed phase HPLC was carried out on a Luna Omega Polar C18 column (50×2.1 mm 1.6 μm) with a flow rate of 0.5 mL/min. Two mobile phases were used, mobile phase A: ammonium formate buffer solution at pH 3.5; mobile phase B: acetonitrile (Chromasolv Sigma-Aldrich), and they were employed to run a gradient conditions from 15% B for 0.2 minutes, from 15% to 95% in 1.6 minutes, 98% B for 0.65 minutes and 15% B in 0.15 minutes and hold these conditions for 1.9 minutes in order to re-equilibrate the column (Total Run Time 4.5 minutes). An injection volume of 0.8 μl was used.

Table 3 shows retention time (Rₜ) in minutes, [M+H]⁺ and/or [M−H]⁻ peak and LCMS procedure.

TABLE 3

| Example | Rₜ (min) | [M + H]⁺ | [M − H]⁻ | LC-MS |
|---|---|---|---|---|
| 1 | 6.47 | 373.3 | 371.4 | 1 |
| 2 | 6.11 | 360.2 | 358.5 | 1 |
| 3 | 5.13 | 384.2 | 382.2 | 1 |
| 4 | 5.48 | 436.0 | 434.4 | 1 |
| 5 | 2.30 | 503.4 | — | 2 |
| 6 | 5.80 | 424.1 | 422.3 | 1 |
| 7 | 5.06 | 388.4 | — | 1 |
| 8 | 5.74 | 458.1 | 456.6 | 1 |
| 9 | 5.86 | 472.3 | 470.6 | 1 |
| 10 | 2.31 | 495.2 | — | 2 |
| 11 | 5.87 | 395.3 | 393.4 | 1 |
| 12 | 6.31 | 500.2 | 498.7 | 1 |
| 13 | 5.77 | 525.0 | 523.6 | 1 |
| 14 | 2.28 | 523.2 | 521.2 | 2 |
| 15 | 5.48 | 402.4 | — | 1 |
| 16 | 6.53 | 391.3 | 389.6 | 1 |
| 17 | 6.08 | 363.2 | 361.5 | 1 |
| 18 | 2.66 | 446.2 | — | 2 |
| 19 | 2.66 | 446.2 | — | 2 |
| 20 | 2.40 | 517.3 | — | 2 |
| 21 | 6.46 | 377.3 | — | 1 |
| 22 | 3.94 | 430.3 | 428.3 | 1 |
| 23 | 6.25 | 331.2 | 329.4 | 1 |
| 24 | 6.12 | 516.1 | 514.6 | 1 |
| 25 | 6.19 | 530.2 | 528.8 | 1 |
| 26 | 5.41 | 454.3 | 542.6 | 1 |
| 27 | 5.09 | 426.3 | 424.5 | 1 |
| 28 | 2.74 | 460.2 | — | 2 |
| 29 | 2.92 | 459.3 | — | 2 |
| 30 | 2.35 | 438.2 | 436.1 | 2 |
| 31 | 2.84 | 475.2 | — | 2 |
| 32 | 5.28 | 410.3 | 408.5 | 1 |
| 33 | 5.58 | 460.2 | 458.6 | 1 |
| 35 | 6.28 | 500.3 | 498.7 | 1 |
| 36 | 5.58 | 454.3 | 542.6 | 1 |
| 37 | 5.88 | 486.4 | 484.7 | 1 |
| 38 | 2.01 | 440.1 | 438.1 | 2 |
| 39 | 2.64 | 375.2 | — | 2 |
| 40 | 2.32 | 468.2 | 466.2 | 2 |
| 41 | 2.32 | 468.2 | 466.2 | 2 |
| 42 | 2.32 | 468.2 | 466.2 | 2 |
| 43 | 2.18 | 440.2 | 438.2 | 2 |
| 44 | 2.31 | 442.2 | 440.1 | 2 |
| 45 | 2.50 | 482.2 | 480.0 | 2 |
| 46 | 2.48 | 531.3 | — | 2 |
| 47 | 2.57 | 432.2 | 430.2 | 2 |
| 49 | 5.44 | 414.4 | — | 1 |
| 50 | 2.65 | 446.2 | 444.2 | 2 |
| 51 | 2.63 | 446.3 | — | 2 |
| 52 | 5.18 | 483.3 | 481.6 | 1 |
| 53 | 5.60 | — | 549.7 | 1 |
| 54 | 2.66 | 446.2 | — | 2 |
| 55 | 4.62 | 506.3 | 504.6 | 1 |
| 56 | 2.42 | 453.2 | 451.1 | 2 |
| 57 | 4.67 | 522.2 | 520.7 | 1 |
| 58 | 2.56 | 436.2 | — | 2 |
| 59 | 2.64 | 452.2 | — | 2 |
| 60 | 2.35 | 433.2 | 431.2 | 2 |
| 61 | 2.71 | 432.6 | 430.4 | 1 |
| 62 | 4.67 | 522.4 | 520.8 | 1 |
| 63 | 4.67 | 522.4 | 520.7 | 1 |
| 64 | 2.45 | 437.2 | 435.1 | 1 |
| 65 | 2.70 | 438.2 | — | 2 |
| 66 | 2.76 | 403.3 | — | 2 |
| 67 | 2.65 | 393.2 | — | 2 |
| 68 | 2.75 | 409.2 | — | 2 |
| 70 | 2.90 | 497.2 | 495.2 | 2 |
| 71 | 2.74 | 481.2 | 479.2 | 2 |
| 72 | 2.59 | 473.2 | — | 2 |
| 73 | 2.86 | 529.3 | 527.2 | 2 |
| 74 | 3.03 | 438.2 | 436.0 | 2 |
| 75 | 3.10 | 517.2 | 515.2 | 2 |
| 76 | 3.21 | 524.3 | 522.3 | 2 |
| 77 | 3.00 | 518.2 | 516.3 | 2 |
| 78 | 2.98 | — | 522.2 | 2 |
| 79 | 2.85 | 533.2 | 535.2 | 2 |
| 80 | 2.70 | 550.2 | 548.3 | 2 |
| 81 | 2.86 | 549.2 | 547.2 | 2 |
| 82 | 2.90 | 524.3 | 522.3 | 2 |
| 83 | 2.56 | 432.2 | 430.3 | 2 |
| 84 | 2.49 | 448.2 | 446.2 | 2 |
| 85 | 2.47 | 438.2 | 436.2 | 2 |
| 86 | 1.98 | 517.2 | 515.2 | 2 |
| 87 | 2.26 | 509.2 | 507.2 | 2 |
| 88 | 2.22 | 521.3 | 523.3 | 2 |
| 89 | 2.24 | 518.3 | 516.3 | 2 |
| 90 | 2.74 | 431.2 | — | 2 |
| 91 | 2.62 | 447.2 | 445.5 | 2 |
| 92 | 2.55 | 474.2 | — | 2 |
| 93 | 2.53 | 474.2 | — | 2 |
| 94 | 2.53 | 474.2 | — | 2 |
| 95 | 2.58 | 483.2 | 481.2 | 2 |
| 96 | 2.33 | 502.3 | — | 2 |
| 97 | 2.69 | 444.1 | — | 2 |
| 98 | 2.35 | 425.2 | 423.2 | 2 |
| 99 | 2.48 | 445.2 | — | 2 |
| 100 | 2.43 | 502.1 | 500.1 | 2 |
| 101 | 2.46 | 517.2 | — | 2 |
| 103 | 2.25 | 495.3 | — | 2 |
| 105 | 2.96 | 482.2 | 480.1 | 2 |
| 106 | 1.82 | 517.2 | — | 2 |

NMR Characterization

¹H NMR spectra were recorded on a Varian Mercury NMR 400 MHz spectrometer using CDCl₃. DMSO-d6 or CD₃OD as solvents Chemical shifts (δ) are reported in parts per million (ppm) relative to residual signal of non-fully deuterated solvents pick for ¹H NMR assigned as 7.26 ppm for CHCl₃, 3.31 ppm for CHD₂OD and 2.50 ppm for DMSO-d₅.

TABLE 4
NMR data of compounds

| Example | Structure | NMR |
|---|---|---|
| 1 | 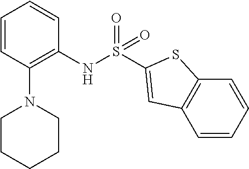 [SMILES: O=S(=O)(Nc1ccccc1N1CCCCC1)c1cc2ccccc2s1] | ¹H NMR (CDCl₃) δ ppm 1.52 (br s, 2 H) 1.64 (quin, J = 5.34 Hz, 4 H) 2.44- 2.53 (m, 4 H) 7.00-7.17 (m, 3 H) 7.41 (quint, J = 7.26, 7.26, 7.26, 7.26, 1.19 Hz, 2 H) 7.70 (dd, J = 8.06, 1.10 Hz, 1 H) 7.74-7.82 (m, 2 H) 7.84 (s, 1 H) 8.38 (NH) |
| 2 | 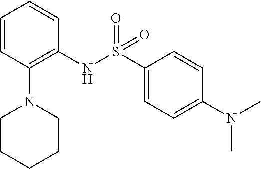 [SMILES: CN(C)c1ccc(S(=O)(=O)Nc2ccccc2N2CCCCC2)cc1] | ¹H NMR (CDCl₃) δ ppm 1.51 (br s, 2 H) 1.55-1.68 (m, 4 H) 2.38-2.52 (m, 4 H) 2.92 (s, 6 H) 6.51 (d, J = 9.07 Hz, 2 H) 6.92-7.07 (br s, 3 H) 7.53-7.63 (br s, 3 H) 7.98 (NH) |
| 3 | 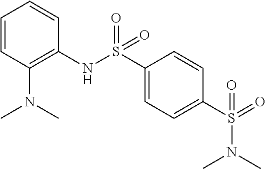 [SMILES: CN(C)c1ccccc1NS(=O)(=O)c1ccc(S(=O)(=O)N(C)C)cc1] | ¹H NMR (CDCl₃) δ ppm 2.40 (s, 7 H) 2.68 (s, 6 H) 6.99-7.16 (m, 3 H) 7.55 (dd, J = 7.93, 1.42 Hz, 1 H) 7.81 (d, J = 8.43 Hz, 2 H) 7.96-8.01 (d, J = 8.43 Hz, 2 H) |
| 4 | 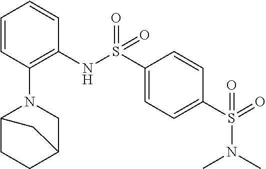 [SMILES: CN(C)S(=O)(=O)c1ccc(S(=O)(=O)Nc2ccccc2N2CC3CCC2C3)cc1] | ¹H NMR (CDCl₃) δ ppm 1.34-1.55 (m, 3 H) 1.60-1.72 (m, 3 H) 2.51 (br s, 1 H) 2.70 (s, 6 H) 2.75 (br s, 1 H) 2.99 (br s, 1 H) 3.24 (br s, 1 H) 6.89- 6.97 (m, 2 H) 7.01-7.07 (m, 1 H) 7.38 (d, J = 8.06 Hz, 1 H) 7.81 (d, J = 8.34 Hz, 2 H) 7.94 (d, J = 8.34 Hz, 2 H) |
| 5 | 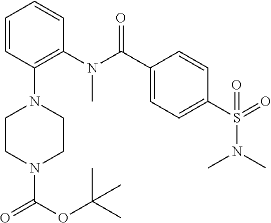 [SMILES: CN(C(=O)c1ccc(S(=O)(=O)N(C)C)cc1)c1ccccc1N1CCN(C(=O)OC(C)(C)C)CC1] | ¹H NMR (CDCl₃) δ ppm 1.40 (s, 9 H) 2.18 (br s, 2 H) 2.54 (s, 6 H) 2.71 (br s, 2 H) 3.29-3.38 (m, 2 H) 3.41 (br dd, J = 6.32, 3.06 Hz, 2 H) 3.45 (s, 3 H) 6.73 (br d, J = 7.50 Hz, 1 H) 7.03-7.15 (m, 2 H) 7.25 (dd, J = 7.63, 1.63 Hz, 1 H) 7.44 (s, 4 H) |
| 6 | 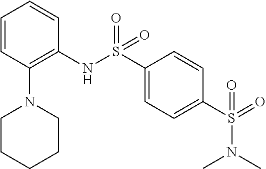 [SMILES: CN(C)S(=O)(=O)c1ccc(S(=O)(=O)Nc2ccccc2N2CCCCC2)cc1] | ¹H NMR (CDCl₃) δ ppm 1.52 (br s, 2 H) 1.57-1.66 (m, 4 H) 2.38-2.46 (m, 4 H) 2.67 (s, 6 H) 7.01-7.13 (m, 3 H) 7.58 (d, J = 7.88 Hz, 1 H) 7.79 (d, J = 8.34 Hz, 2 H) 7.95 (d, J = 8.34 Hz, 2 H) |
| 7 | 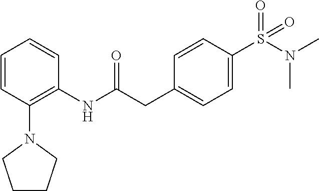 [SMILES: CN(C)S(=O)(=O)c1ccc(CC(=O)Nc2ccccc2N2CCCC2)cc1] | ¹H NMR (CDCl₃) δ ppm 1.76 (br s, 4 H) 2.70 (s, 6 H) 2.77 (br s, 4 H) 3.82 (s, 2 H) 6.97-7.10 (br s, 3 H) 7.53 (br d, J = 8.06 Hz, 2 H) 7.77 (d, J = 8.06 Hz, 2 H) 8.19-8.26 (m, 1 H) 8.31 (NH) |
| 8 | 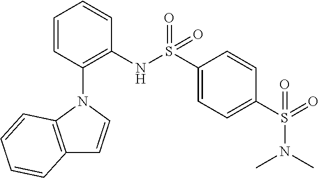 [SMILES: CN(C)S(=O)(=O)c1ccc(S(=O)(=O)Nc2ccccc2-n2ccc3ccccc32)cc1] | ¹H NMR (CDCl₃) δ ppm 2.71 (s, 6 H) 3.03-3.15 (t, 2 H) 3.36-3.45 (t, 2 H) 5.68 (d, J = 7.79 Hz, 1 H) 6.74-6.81 (m, 1 H) 6.87-6.93 (m, 1 H) 7.11- 7.20 (m, 3 H) 7.23-7.31 (m, 1 H) 7.65-7.69 (m, 1 H) 7.73 (d, J = 8.34 Hz, 1 H) 7.80 (d, J = 8.52 Hz, 2 H) 7.92 (d, J = 8.43 Hz, 2 H) |
| 9 | 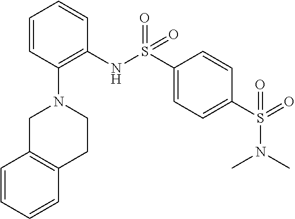 [SMILES: CN(C)S(=O)(=O)c1ccc(S(=O)(=O)Nc2ccccc2N2CCc3ccccc3C2)cc1] | ¹H NMR (CDCl₃) δ ppm 2.72 (s, 6 H) 2.84-2.92 (m, 2 H) 2.92-2.98 (m, 2 H) 3.70 (s, 2 H) 6.92 (br d, J = 7.15 Hz, 1 H) 7.10-7.25 (m, 6 H) 7.63-7.69 (m, 1 H) 7.83 (d, J = 8.34 Hz, 2 H) 7.95 (d, J = 8.43 Hz, 2 H) 8.17 (NH) |
| 10 | 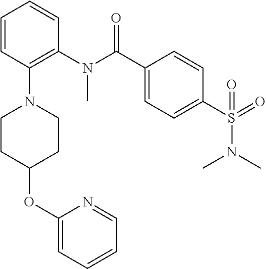 [SMILES: CN(C(=O)c1ccc(S(=O)(=O)N(C)C)cc1)c1ccccc1N1CCC(Oc2ccccn2)CC1] | ¹H NMR (CDCl₃) δ ppm 1.63-1.75 (m, 1 H) 1.81-1.89 (m, 1 H) 1.97 (m, 2 H) 2.24-2.44 (m, 2 H) 2.53 (s, 6 H) 2.70-2.82 (m, 1 H) 2.99 (br s, 1 H) 3.46 (s, 3 H) 5.06 (tt, J = 7.99, 3.83 Hz, 1 H) 6.66 (d, J = 8.38 Hz, 1 H) 6.74- 6.83 (m, 2 H) 6.99-7.05 (m, 1 H) 7.09-7.15 (m, 1 H) 7.22 (dd, J = 7.75, 1.50 Hz, 1 H) 7.42-7.59 (m, 5 H) 8.05 (dd, J = 5.00, 1.38 Hz, 1 H) |
| 11 | 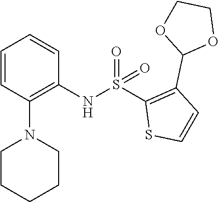 [SMILES: O=S(=O)(Nc1ccccc1N1CCCCC1)c1sccc1C1OCCO1] | ¹H NMR (CDCl₃) δ ppm 1.54 (br s, 2 H) 1.64-1.72 (m, 4 H) 2.59-2.69 (m, 4 H) 3.96-4.07 (m, 4 H) 6.25 (s, 1 H) 6.97-7.14 (m, 3 H) 7.17 (d, J = 5.13 Hz, 1 H) 7.39 (d, J = 5.13 Hz, 1 H) 7.64 (dd, J = 7.97, 1.01 Hz, 1 H) 8.50 (NH) |
| 12 | 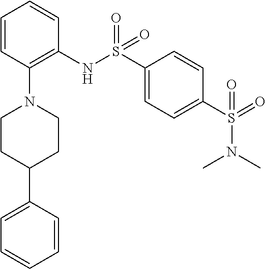 [SMILES: CN(C)S(=O)(=O)c1ccc(S(=O)(=O)Nc2ccccc2N2CCC(c3ccccc3)CC2)cc1] | ¹H NMR (CDCl₃) δ ppm 1.76-1.95 (m, 4 H) 2.48-2.63 (m, 4 H) 2.65- 2.76 (br s, 7 H) 7.06-7.11 (m, 1 H) 7.12-7.19 (m, 2 H) 7.23-7.30 (m, 3 H) 7.33-7.39 (m, 2 H) 7.62 (br d, J = 7.97 Hz, 1 H) 7.80 (d, J = 8.25 Hz, 2 H) 7.98 (br d, J = 8.25 Hz, 2 H) 8.18 (NH) |
| 13 | 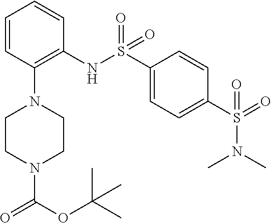 [SMILES: CN(C)S(=O)(=O)c1ccc(S(=O)(=O)Nc2ccccc2N2CCN(C(=O)OC(C)(C)C)CC2)cc1] | ¹H NMR (CDCl₃) δ ppm 1.48 (s, 9 H) 2.54 (br s, 4 H) 2.72 (s, 6 H) 3.52 (br s, 4 H) 7.06-7.12 (m, 2 H) 7.13-7.19 (m, 1 H) 7.56 (d, J = 7.97 Hz, 1 H) 7.84 (d, J = 8.43 Hz, 2 H) 7.98 (d, J = 8.34 Hz, 2H) 8.12 (NH) |
| 14 | 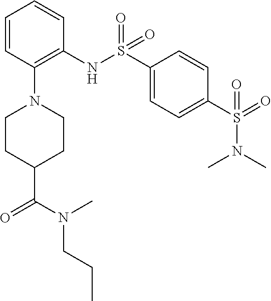 [SMILES: CCCN(C)C(=O)C1CCN(c2ccccc2NS(=O)(=O)c2ccc(S(=O)(=O)N(C)C)cc2)CC1] | ¹H NMR (CD₃OD) 5 ppm 0.88-0.99 (m, 3 H) 1.54-1.73 (m, 4 H) 1.77- 1.94 (m, 2 H) 2.41 (td, J = 7.63, 3.75 Hz, 2 H) 2.58-2.65 (m, 2 H) 2.67 (s, 6H) 2.72-2.82 (m, 1 H) 3.11 (s, 3 H) 3.33 (dt, J = 3.25, 1.63 Hz, 1 H) 3.35- 3.44 (m, 2 H) 7.08-7.23 (m, 3 H) 7.59 (dd, J = 7.57, 1.81 Hz, 1 H) 7.90 (d, J = 8.38 Hz, 2 H) 8.03 (d, J = 8.38 Hz, 2 H) |
| 15 | 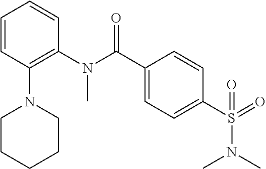 [SMILES: CN(C(=O)c1ccc(S(=O)(=O)N(C)C)cc1)c1ccccc1N1CCCCC1] | ¹H NMR (CDCl₃) δ ppm 1.44-1.66 (br s, 6 H) 2.23 (m, 2 H) 2.58 (s, 6 H) 2.75 (m, 2 H) 3.51 (s, 3 H) 6.78 (br d, J = 7.88 Hz, 1 H) 7.00-7.08 (m, 1 H) 7.10-7.17 (m, 1 H) 7.22-7.28 (m, 1 H) 7.49 (br s, 4 H) |
| 16 | 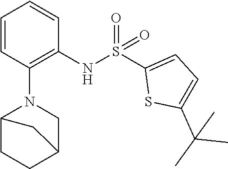 [SMILES: CC(C)(C)c1ccc(S(=O)(=O)Nc2ccccc2N2CC3CCC2C3)s1] | ¹H NMR (CDCl₃) δ ppm 1.32 (s, 9 H) 1.35-1.52 (br s, 3 H) 1.60-1.72 (br s, 2 H) 1.75 (br d, J = 9.62 Hz, 1 H) 2.51 (br s, 1 H) 2.80 (d, J = 8.89 Hz, 1 H) 3.02 (dt, J = 8.77, 2.90 Hz, 1 H) 3.22 (br s, 1 H) 6.71 (d, J = 3.85 Hz, 1 H) 6.92-7.06 (m, 3 H) 7.31-7.35 (m, 1 H) 7.32 (d, J = 3.85 Hz, 1 H) 7.49- 7.53 (m, 1 H) |
| 17 | 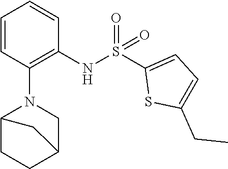 [SMILES: CCc1ccc(S(=O)(=O)Nc2ccccc2N2CC3CCC2C3)s1] | ¹H NMR (CDCl₃) δ ppm 1.25 (t, J = 7.51 Hz, 3 H) 1.36-1.54 (br s, 3 H) 1.60-1.73 (br s, 2 H) 1.76 (br d, J = 9.62 Hz, 1 H) 2.52 (br s, 1 H) 2.75- 2.87 (br s, 3 H) 3.03 (dt, J = 8.80, 2.89 Hz, 1 H) 3.28 (br s, 1 H) 6.68 (d, J = 3.76 Hz, 1 H) 6.92-7.05 (m, 3 H) 7.36 (d, J = 3.76 Hz, 1 H) 7.49 (dd, J = 7.84, 1.15 Hz, 1 H) |
| 18 | 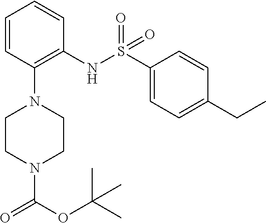 [SMILES: CCc1ccc(S(=O)(=O)Nc2ccccc2N2CCN(C(=O)OC(C)(C)C)CC2)cc1] | ¹H NMR (CDCl₃) δ ppm 1.12 (t, J = 7.63 Hz, 3 H) 1.40 (s, 9 H) 2.33 (br s, 4 H) 2.57 (q, J = 7.63 Hz, 2 H) 3.37 (br s, 4 H) 6.97 (d, J = 4.00 Hz, 2 H) 7.04-7.09 (m, 1 H) 7.13-7.20 (m, 2 H) 7.57 (d, J = 8.00 Hz, 1 H) 7.62 (d, J = 8.38 Hz, 2 H) 7.79 (NH) |
| 19 | 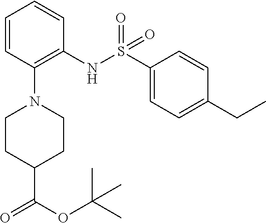 [SMILES: CCc1ccc(S(=O)(=O)Nc2ccccc2N2CCC(C(=O)OC(C)(C)C)CC2)cc1] | ¹H NMR (CDCl₃) δ ppm 1.11 (t, J = 7.63 Hz, 3 H) 1.41 (s, 9 H) 1.60- 1.75 (m, 2 H) 1.81 (br dd, J = 13.26, 3.25 Hz, 2 H) 2.20 (tt, J = 10.85, 4.03 Hz, 1 H) 2.37-2.43 (m, 4 H) 2.57 (q, J = 7.59 Hz, 2 H) 6.89-6.99 (m, 2 H) 7.03 (td, J = 7.50, 1.88 Hz, 1 H) 7.15 (d, J = 8.25 Hz, 2 H) 7.55 (dd, J = 8.13, 1.13 Hz, 1 H) 7.63 (d, J = 8.38 Hz, 2 H) 7.84 (NH) |
| 20 | 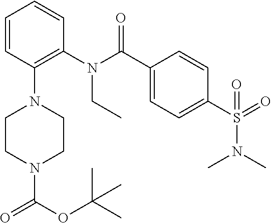 [SMILES: CCN(C(=O)c1ccc(S(=O)(=O)N(C)C)cc1)c1ccccc1N1CCN(C(=O)OC(C)(C)C)CC1] | ¹H NMR (CDCl₃) δ ppm 1.36 (br s, 3 H) 1.41 (s, 9 H) 1.68 (m, 2 H) 2.54 (s, 6 H) 2.73 (m, 2 H) 3..5-3.42 (br s, 5 H) 4.42 (br s, 1 H) 6.77 (m, 1 H) 6.98- 7.05 (m, 1 H) 7.09-7.15 (m, 1 H) 7.18-7.25 (m, 1 H) 7.22-7.45 (m, 4 H) |
| 21 | 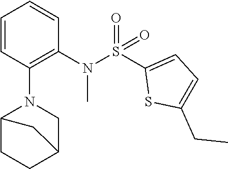 [SMILES: CCc1ccc(S(=O)(=O)N(C)c2ccccc2N2CC3CCC2C3)s1] | ¹H NMR (CDCl₃) δ ppm 1.30-1.39 (br s, 5 H) 1.41-1.49 (m, 2 H) (m, 2 H) 2.57 (br s, 2 H) 2.75 (br d, J = 8.71 Hz, 1 H) 2.85-2.95 (m, 2 H) 3.20 (s, 3 H) 3.72 (m, 1 H) 6.42-6.53 (m, 2 H) 6.70 (br d, J = 8.43 Hz, 1 H) 6.80 (br d, J = 3.39 Hz, 1 H) 7.06-7.14 (m, 1 H) 7.21 (d, J = 3.57 Hz, 1 H) |
| 22 | 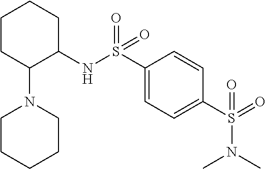 [SMILES: CN(C)S(=O)(=O)c1ccc(S(=O)(=O)NC2CCCCC2N2CCCCC2)cc1] | ¹H NMR (CDCl₃) δ ppm 0.97-1.27 (m, 4 H) 1.30-1.50 (m, 6 H) 1.64 (br d, J = 9.16 Hz, 1 H) 1.76 (br t, J = 13.38 Hz, 2 H) 2.13 (br s, 5 H) 2.37 (br d, J = 12.83 Hz, 1 H) 2.66-2.72 (m, 1 H) 2.73 (s, 6 H) 7.90 (d, J = 8.43 Hz, 2 H) 8.04 (d, J = 8.43 Hz, 2 H) |
| 23 | 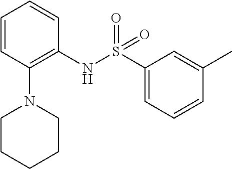 [SMILES: Cc1cccc(S(=O)(=O)Nc2ccccc2N2CCCCC2)c1] | ¹H NMR (CDCl₃) δ ppm 1.51 (m, 2 H) 1.61 (quin, J = 5.27 Hz, 4 H) 2.31 (s, 3 H) 2.38-2.43 (m, 4 H) 6.96- 7.11 (br s, 3 H) 7.21-7.29 (m, 2 H) 7.56 (br d, J = 7.15 Hz, 1 H) 7.59-7.64 (m, 2 H) 8.09 (NH) |
| 24 | 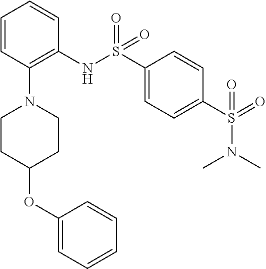 [SMILES: CN(C)S(=O)(=O)c1ccc(S(=O)(=O)Nc2ccccc2N2CCC(Oc3ccccc3)CC2)cc1] | ¹H NMR (CDCl₃) δ ppm 1.91 (br s, 2 H) 2.02 (br s, 2 H) 2.47 (br s, 2 H) 2.67 (s, 6 H) 2.73 (br s, 2 H) 4.45 (br s, 1 H) 6.92 (d, J = 8.06 Hz, 2 H) 6.94- 6.99 (m, 1 H) 7.05-7.11 (m, 1 H) 7.11-7.19 (m, 2 H) 7.30 (t, J = 7.88 Hz, 2 H) 7.60 (br d, J = 7.79 Hz, 1 H) 7.81 (br d, J = 8.25 Hz, 2 H) 7.97 (br d, J = 8.25 Hz, 2H) 8.13 (NH) |
| 25 | 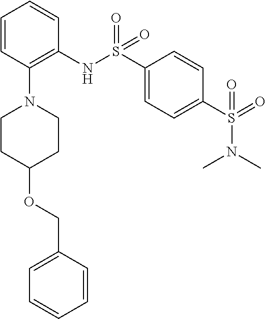 [SMILES: CN(C)S(=O)(=O)c1ccc(S(=O)(=O)Nc2ccccc2N2CCC(OCc3ccccc3)CC2)cc1] | ¹H NMR (CDCl₃) δ ppm 1.74 (m, 2 H) 1.94 (m, 2 H) 2.42 (m, 2 H) 2.53- 2.65 (m, 2 H) 2.69 (s, 6 H) 3.54 (m, 1 H) 4.56 (s, 2 H) 7.09 (tt, J = 14.25, 7.29 Hz, 3 H) 7.27-7.33 (m, 1 H) 7.33- 7.42 (m, 4 H) 7.60 (br d, J = 7.79 Hz, 1 H) 7.80 (br d, J = 8.06 Hz, 2 H) 7.96 (br d, J = 8.06 Hz, 2 H) 8.12 (NH) |
| 26 | 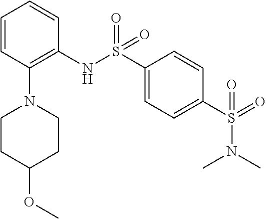 [SMILES: COC1CCN(c2ccccc2NS(=O)(=O)c2ccc(S(=O)(=O)N(C)C)cc2)CC1] | ¹H NMR (CDCl₃) δ ppm 1.64 (m, 2 H) 1.89 (m, 2 H) 2.39 (m, 2 H) 2.55 (m, 2 H) 2.67 (s, 6 H) 3.33 (br s, 4 H) 6.99-7.14 (m, 3 H) 7.57 (br d, J = 7.79 Hz, 1 H) 7.79 (br d, J = 8.25 Hz, 2 H) 7.94 (br d, J = 8.25 Hz, 2 H) 8.11 (NH) |
| 27 | 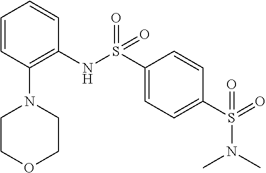 [SMILES: CN(C)S(=O)(=O)c1ccc(S(=O)(=O)Nc2ccccc2N2CCOCC2)cc1] | ¹H NMR (CDCl₃) δ ppm 2.56 (br s, 4 H) 2.70 (s, 6 H) 3.72-3.80 (m, 4 H) 7.06-7.17 (br s, 3 H) 7.55 (br d, J = 7.97 Hz, 1 H) 7.82 (d, J = 8.43 Hz, 2 H) 7.98 (d, J = 8.43 Hz, 2 H) 8.09 (NH) |
| 28 | 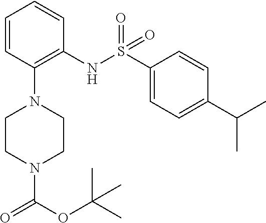 [SMILES: CC(C)c1ccc(S(=O)(=O)Nc2ccccc2N2CCN(C(=O)OC(C)(C)C)CC2)cc1] | ¹H NMR (CDCl₃) δ ppm 1.21 (d, J = 6.88 Hz, 6 H) 1.49 (s, 9 H) 2.38 (br s, 4 H) 2.83-2.99 (m, 1 H) 3.44 (br s, 4 H) 7.05-7.09 (m, 2 H) 7.17 (ddd, J = 8.32, 5.44, 3.25 Hz, 1 H) 7.27 (d, J = 8.50 Hz, 2 H) 7.66 (d, J = 8.13 Hz, 1 H) 7.70 (d, J = 8.38 Hz, 2 H) 7.86 (NH) |
| 29 | 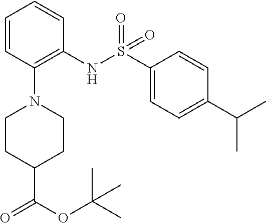 [SMILES: CC(C)c1ccc(S(=O)(=O)Nc2ccccc2N2CCC(C(=O)OC(C)(C)C)CC2)cc1] | ¹H NMR (CDCl₃) δ ppm 1.12 (d, J = 6.88 Hz, 6 H) 1.40 (s, 9 H) 1.58- 1.71 (m, 2 H) 1.76-1.84 (m, 2 H) 2.19 (tt, J = 10.79, 4.10 Hz, 1 H) 2.36 (dd, J = 7.63, 2.50 Hz, 4 H) 2.82 (spt, J = 6.90 Hz, 1 H) 6.89-7.01 (m, 2 H) 7.01-7.09 (m, 1 H) 7.15-7.23 (m, 2 H) 7.56 (dd, J = 8.07, 0.94 Hz, 1 H) 7.63 (d, J = 8.38 Hz, 2 H) 7.81 (NH) |
| 30 | 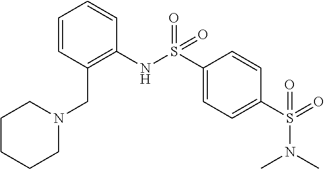 [SMILES: CN(C)S(=O)(=O)c1ccc(S(=O)(=O)Nc2ccccc2CN2CCCCC2)cc1] | ¹H NMR (CDCl₃) δ ppm 1.50 (br s, 2 H) 1.65 (quin, J = 5.53 Hz, 4 H) 2.38 (br s, 4 H) 2.66-2.75 (m, 6 H) 3.23 (s, 2 H) 6.89-7.05 (m, 2 H) 7.15- 7.26 (m, 1 H) 7.45 (d, J = 8.00 Hz, 1 H) 7.79-7.88 (m, 2 H) 7.93-8.00 (m, 2H) |
| 31 | 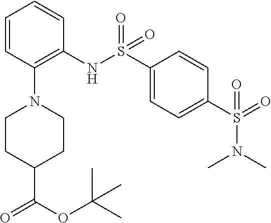 [SMILES: CN(C)S(=O)(=O)c1ccc(S(=O)(=O)Nc2ccccc2N2CCC(C(=O)OC(C)(C)C)CC2)cc1] | ¹H NMR (CDCl₃) δ ppm 1.23 (d, J = 6.00 Hz, 6 H) 1.41 (s, 9 H) 1.63- 1.77 (m, 2 H) 1.84 (br dd, J = 13.26, 3.25 Hz, 2 H) 2.21 (tt, J = 10.85, 4.03 Hz, 1 H) 2.36-2.52 (m, 4 H) 4.48 (dt, J = 12.10, 6.02 Hz, 1 H) 6.72-6.80 (m, 2 H) 6.90-7.06 (m, 3 H) 7.54 (dd, J = 8.07, 1.19 Hz, 1 H) 7.60-7.67 (m, 2 H) 7.82 (NH) |
| 32 | 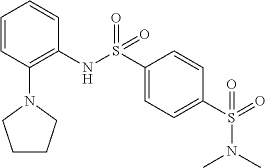 [SMILES: CN(C)S(=O)(=O)c1ccc(S(=O)(=O)Nc2ccccc2N2CCCC2)cc1] | ¹H NMR (CDCl₃) δ ppm 1.88 (dt, J = 6.37, 3.23 Hz, 4 H) 2.70 (s, 6 H) 2.77 (br t, J = 6.37 Hz, 4 H) 6.99-7.09 (br s, 3 H) 7.44-7.50 (m, 1 H) 7.81 (d, J = 8.43 Hz, 2 H) 7.95 (d, J = 8.43 Hz, 2 H) |
| 33 | 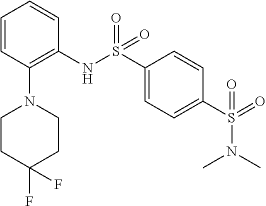 [SMILES: CN(C)S(=O)(=O)c1ccc(S(=O)(=O)Nc2ccccc2N2CCC(F)(F)CC2)cc1] | ¹H NMR (CDCl₃) δ ppm 2.01-2.14 (m, 4 H) 2.67 (m, 4 H) 2.71 (s, 6 H) 7.04-7.19 (br s, 3 H) 7.54 (d, J = 8.25 Hz, 1 H) 7.84 (d, J = 8.43 Hz, 2 H) 7.95 (NH) 7.99 (d, J = 8.43 Hz, 2 H) |
| 35 | 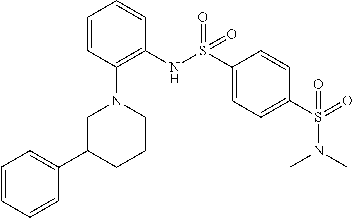 [SMILES: CN(C)S(=O)(=O)c1ccc(S(=O)(=O)Nc2ccccc2N2CCCC(c3ccccc3)C2)cc1] | ¹H NMR (CDCl₃) δ ppm 1.49-1.65 (m, 2 H) 1.70-1.91 (m, 2 H) 2.00- 2.08 (m, 1 H) 2.47-2.53 (m, 1 H) 2.58-2.63 (br s, 8 H) 2.84-2.94 (m, 1 H) 7.02-7.07 (m, 1 H) 7.09-7.15 (m, 2 H) 7.19 (br d, J = 7.15 Hz, 2 H) 7.22-7.26 (m, 1 H) 7.28-7.36 (m, 2 H) 7.58-7.64 (m, 1 H) 7.70 (d, J = 8.34 Hz, 2 H) 7.95 (d, J = 8.34 Hz, 2 H) 8.17 (NH) |
| 36 | 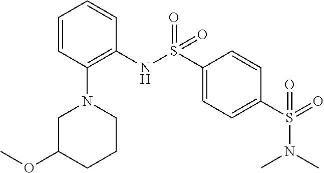 | ¹H NMR (CDCl₃) δ ppm 1.49-1.65 (m, 2 H) 1.82 (m, 2 H) 2.52-2.63 (m, 3 H) 2.67 (br s, 7 H) 3.38 (m, 1 H) 3.46 (s, 3 H) 6.91-7.09 (br s, 3 H) 7.60 (d, J = 7.88 Hz, 1 H) 7.78 (d, J = 8.25 Hz, 2 H) 7.97 (d, J = 8.25 Hz, 2 H) 8.76 (NH) |
| 37 | 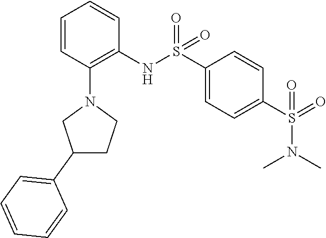 [SMILES: CN(C)S(=O)(=O)c1ccc(S(=O)(=O)Nc2ccccc2N2CCC(c3ccccc3)C2)cc1] | ¹H NMR (CDCl₃) δ ppm 2.02-2.13 (m, 1 H) 2.33-2.44 (m, 1 H) 2.64 (s, 6 H) 2.68-2.75 (m, 1 H) 3.00-3.14 (m, 3 H) 3.37-3.47 (m, 1 H) 7.01- 7.15 (br s, 3 H) 7.23-7.31 (br s, 3 H) 7.34-7.41 (m, 2 H) 7.51 (dd, J = 7.84, 1.24 Hz, 1 H) 7.70-7.75 (m, 2 H) 7.78-7.83 (m, 2 H) |
| 38 | 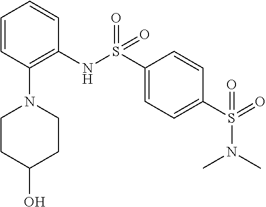 [SMILES: CN(C)S(=O)(=O)c1ccc(S(=O)(=O)Nc2ccccc2N2CCC(O)CC2)cc1] | ¹H NMR (CDCl₃) δ ppm 1.60-1.74 (m, 2 H) 1.95 (br d, J = 9.38 Hz, 2 H) 2.43-2.53 (m, 2 H) 2.61 (br d, J = 10.63 Hz, 2 H) 2.72 (s, 6 H) 3.86 (br d, J = 3.38 Hz, 1 H) 7.03-7.20 (m, 3 H) 7.60 (br d, J = 7.88 Hz, 1 H) 7.83 (br d, J = 8.25 Hz, 2 H) 7.99 (br d, J = 8.25 Hz, 2 H) |
| 39 | 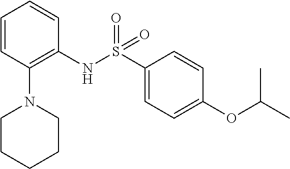 [SMILES: CC(C)Oc1ccc(S(=O)(=O)Nc2ccccc2N2CCCCC2)cc1] | ¹H NMR (CDCl₃) δ ppm 1.23 (d, J = 6.13Hz, 6H) 1.45 (brd, J = 3.38Hz, 2 H) 1.55 (quin, J = 5.32 Hz, 4 H) 2.32- 2.46 (m, 4 H) 4.48 (spt, J = 6.05 Hz, 1 H) 6.75 (d, J = 8.88 Hz, 2 H) 6.88- 6.98 (m, 1 H) 6.98-7.05 (m, 2 H) 7.49-7.55 (m, 1 H) 7.63 (d, J = 8.88 Hz, 2 H) 7.96 (NH) |
| 40 | 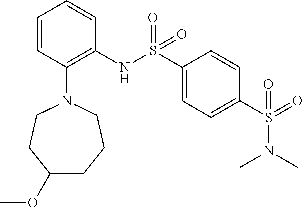 | ¹H NMR (CDCl₃) δ ppm 1.47 (br t, J = 10.94 Hz, 1 H) 1.57-1.68 (m, 2 H) 1.72-1.81 (m, 1 H) 1.84-1.93 (m, 1 H) 1.93-2.03 (m, 1 H) 2.55-2.61 (m, 2 H) 2.62 (s, 6 H) 2.73 (br t, J = 4.94 Hz, 2 H) 3.40 (s, 3 H) 3.62 (dt, J = 5.85, 2.89 Hz, 1 H) 6.85-7.07 (m, 3 H) 7.57 (d, J = 8.00 Hz, 1 H) 7.73 (d, J = 8.38 Hz, 2 H) 7.94 (d, J = 8.38 Hz, 2 H) 8.87 (NH) |
| 41 | 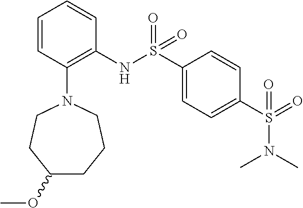 | ¹H NMR (CDCl₃) δ ppm 1.47 (br t, J = 10.94 Hz, 1 H) 1.57-1.68 (m, 2 H) 1.72-1.81 (m, 1 H) 1.84-1.93 (m, 1 H) 1.93-2.03 (m, 1 H) 2.55-2.61 (m, 2 H) 2.62 (s, 6 H) 2.73 (br t, J = 4.94 Hz, 2 H) 3.40 (s, 3 H) 3.62 (dt, J = 5.85, 2.89 Hz, 1 H) 6.85-7.07 (m, 3 H) 7.57 (d, J = 8.00 Hz, 1 H) 7.73 (d, J = 8.38 Hz, 2 H) 7.94 (d, J = 8.38 Hz, 2 H) 8.87 (NH) |
| 42 | 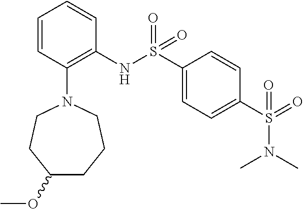 | ¹H NMR (CDCl₃) δ ppm 1.47 (br t, J = 10.94 Hz, 1 H) 1.57-1.68 (m, 2 H) 1.72-1.81 (m, 1 H) 1.84-1.93 (m, 1 H) 1.93-2.03 (m, 1 H) 2.55-2.61 (m, 2 H) 2.62 (s, 6 H) 2.73 (br t, J = 4.94 Hz, 2 H) 3.40 (s, 3 H) 3.62 (dt, J = 5.85, 2.89 Hz, 1 H) 6.85-7.07 (m, 3 H) 7.57 (d, J = 8.00 Hz, 1 H) 7.73 (d, J = 8.38 Hz, 2 H) 7.94 (d, J = 8.38 Hz, 2 H) 8.87 (NH) |
| 43 | 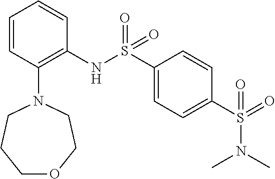 [SMILES: CN(C)S(=O)(=O)c1ccc(S(=O)(=O)Nc2ccccc2N2CCCOCC2)cc1] | ¹H NMR (CDCl₃) δ ppm 1.93 (quin, J = 5.91 Hz, 2 H) 2.72 (s, 6 H) 2.75- 2.80 (m, 2 H) 2.80-2.86 (m, 2 H) 3.69-3.80 (m, 2 H) 3.90 (t, J = 6.19 Hz, 2 H) 7.03-7.18 (m, 3 H) 7.58 (dd, J = 7.88, 1.38 Hz, 1 H) 7.83 (d, J = 8.50 Hz, 2 H) 7.99 (d, J = 8.50 Hz, 2 H) 8.19 (NH) |
| 44 | 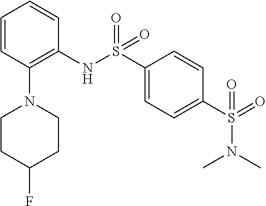 [SMILES: CN(C)S(=O)(=O)c1ccc(S(=O)(=O)Nc2ccccc2N2CCC(F)CC2)cc1] | ¹H NMR (CDCl₃) δ ppm 1.79-1.94 (m, 4 H) 2.28-2.37 (m, 2 H) 2.63 (s, 6 H) 2.64-2.74 (m, 2 H) 4.62-4.85 (m, 1 H) 6.97-7.11 (m, 3 H) 7.46- 7.53 (m, 1 H) 7.76 (d, J = 8.50 Hz, 2 H) 7.91 (d, J = 8.50 Hz, 2 H) 8.01 (NH) |
| 45 | 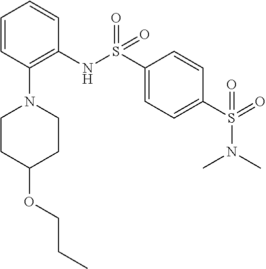 [SMILES: CCCOC1CCN(c2ccccc2NS(=O)(=O)c2ccc(S(=O)(=O)N(C)C)cc2)CC1] | ¹H NMR (CDCl₃) δ ppm 0.96 (t, J = 7.38 Hz, 3 H) 1.55-1.74 (m, 4 H) 1.94 (br d, J = 9.88 Hz, 2 H) 2.44 (br t, J = 9.07 Hz, 2 H) 2.54-2.64 (m, 2 H) 2.68-2.76 (m, 6 H) 3.39-3.47 (m, 3 H) 7.03-7.21 (m, 3 H) 7.62 (dd, J = 7.88, 1.00 Hz, 1 H) 7.82 (d, J = 8.50 Hz, 2 H) 7.97 (d, J = 8.50 Hz, 2 H) 8.14 (NH) |
| 46 | 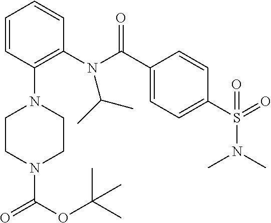 [SMILES: CC(C)N(C(=O)c1ccc(S(=O)(=O)N(C)C)cc1)c1ccccc1N1CCN(C(=O)OC(C)(C)C)CC1] | ¹H NMR (CDCl₃) δ ppm 1.47 (d, J = 6.13 Hz, 6 H) 1.49 (s, 9 H) 2.65 (s, 6 H) 2.79 (br s, 4 H) 3.41 (br s, 4 H) 5.37-5.51 (m, 1 H) 6.72 (d, J = 7.25 Hz, 1 H) 6.81 (d, J = 7.63 Hz, 1 H) 6.90- 7.02 (m, 2 H) 7.48 (br d, J = 8.13 Hz, 2 H) 7.59 (d, J = 8.38 Hz, 2 H) |
| 47 | 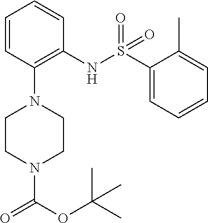 [SMILES: Cc1ccccc1S(=O)(=O)Nc1ccccc1N1CCN(C(=O)OC(C)(C)C)CC1] | ¹H NMR (CDCl₃) δ ppm 1.51 (s, 9 H) 2.67 (s, 3 H) 2.71 (t, J = 4.94 Hz, 4 H) 3.59 (br s, 4 H) 6.96-7.14 (m, 3 H) 7.27-7.35 (m, 2 H) 7.39 (dd, J = 7.94, 1.44 Hz, 1 H) 7.42-7.49 (m, 1 H) 8.01-8.07 (m, 1H) 8.10 (s, 1 H) |
| 49 | 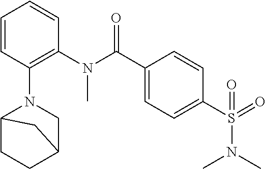 [SMILES: CN(C(=O)c1ccc(S(=O)(=O)N(C)C)cc1)c1ccccc1N1CC2CCC1C2] | ¹H NMR (CDCl₃) δ ppm 1.35 (m, 1 H) 1.51-1.70 (br s, 4 H) 1.78-1.87 (m, 1 H) 2.45 (d, J = 8.06 Hz, 1 H) 2.57 (s, 6 H) 3.34-3.42 (m, 2 H) 3.47 (s, 3 H) 3.57 (dt, J = 7.81, 2.97 Hz, 1 H) 6.34 (d, J = 8.25 Hz, 1 H) 6.70 (t, J = 7.51 Hz, 1 H) 6.94-7.00 (m, 1 H) 7.15 (dd, J = 7.79, 1.37 Hz, 1 H) 7.33 (d, J = 8.34 Hz, 2 H) 7.45 (d, J = 8.34 Hz, 2 H) |
| 50 | 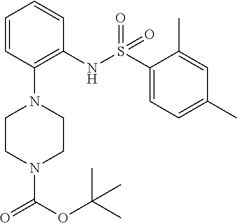 [SMILES: Cc1ccc(S(=O)(=O)Nc2ccccc2N2CCN(C(=O)OC(C)(C)C)CC2)c(C)c1] | ¹H NMR (CDCl₃) δ ppm 1.51 (s, 9 H) 2.35 (s, 3 H) 2.62 (s, 3 H) 2.71 (t, J = 4.88 Hz, 4 H) 3.59 (br s, 4 H) 6.97- 7.06 (m, 2 H) 7.06-7.13 (m, 3 H) 7.38 (dd, J = 8.00, 1.25 Hz, 1 H) 7.93 (d, J = 8.00 Hz, 1 H) 8.07 (NH) |
| 51 | 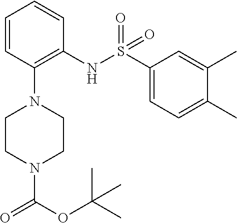 [SMILES: Cc1ccc(S(=O)(=O)Nc2ccccc2N2CCN(C(=O)OC(C)(C)C)CC2)cc1C] | ¹H NMR (CDCl₃) δ ppm 1.50 (s, 9 H) 2.27 (d, J = 3.88 Hz, 6 H) 2.38-2.59 (m, 4 H) 3.49 (br s, 4 H) 7.00-7.12 (m, 2 H) 7.12-7.21 (m, 2 H) 7.51 (br d, J = 7.88 Hz, 1 H) 7.60 (s, 1 H) 7.63 (d, J = 8.13 Hz, 1 H) 7.89 (s, NH) |
| 52 | 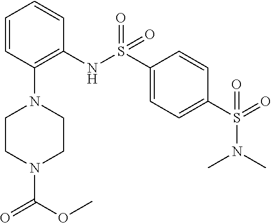 [SMILES: COC(=O)N1CCN(c2ccccc2NS(=O)(=O)c2ccc(S(=O)(=O)N(C)C)cc2)CC1] | ¹H NMR (CDCl₃) δ ppm 2.50 (m, 4 H) 2.71 (s, 6 H) 3.60 (m, 4 H) 4.11 (s, 3 H) 7.04-7.12 (m, 2 H) 7.14-7.30 (m, 1 H) 7.60 (d, J = 7.80 Hz, 1 H) 7.89 (d, J = 8.44, 2 H) 8.05 (d, J = 8.44 Hz, 2 H) 8.10 (NH) |
| 53 | 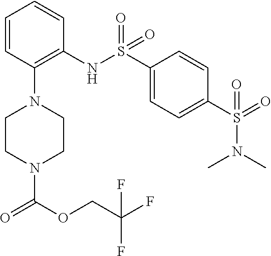 [SMILES: CN(C)S(=O)(=O)c1ccc(S(=O)(=O)Nc2ccccc2N2CCN(C(=O)OCC(F)(F)F)CC2)cc1] | ¹H NMR (CDCl₃) δ ppm 2.51 (br s, 4 H) 2.65 (s, 6 H) 3.54 (br s, 4 H) 4.45 (q, J = 8.50 Hz, 2 H) 6.98-7.06 (m, 2 H) 7.06-7.14 (m, 1 H) 7.48 (d, J = 7.75 Hz, 1 H) 7.78 (d, J = 8.50 Hz, 2 H) 7.93 (d, J = 8.50 Hz, 2 H) |
| 54 | 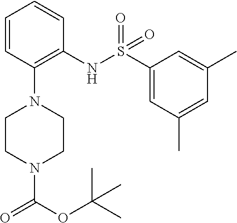 [SMILES: Cc1cc(C)cc(S(=O)(=O)Nc2ccccc2N2CCN(C(=O)OC(C)(C)C)CC2)c1] | ¹H NMR (400 MHz, CHLOROFORM-d) δ ppm 1.50 (s, 9 H) 2.31 (s, 6 H) 2.46 (br s, 4 H) 3.50 (br s, 4 H) 7.04-7.09 (m, 2 H) 7.13- 7.20 (m, 2 H) 7.42 (s, 2 H) 7.64 (d, J = 8.00 Hz, 1 H) 7.89 (NH) |
| 55 | 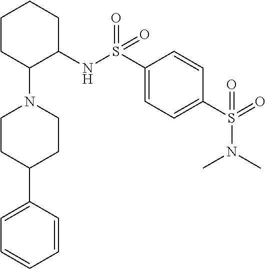 [SMILES: CN(C)S(=O)(=O)c1ccc(S(=O)(=O)NC2CCCCC2N2CCC(c3ccccc3)CC2)cc1] | ¹H NMR (CDCl₃) δ ppm 1.06-1.39 (m, 6 H) 1.67 (br d, J = 12.10 Hz, 2 H) 1.76-1.92 (m, 4 H) 2.08 (br s, 1 H) 2.25-2.47 (m, 3 H) 2.62-2.68 (m, 2 H) 2.70 (s, 6 H) 2.80 (br d, J = 6.42 Hz, 1 H) 7.16-7.24 (br s, 3 H) 7.29-7.35 (m, 2 H) 7.90 (d, J = 8.34 Hz, 2 H) 8.07 (d, J = 8.34 Hz, 2 H) |
| 56 | 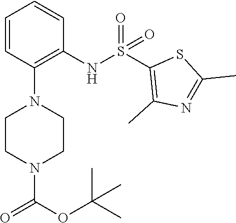 [SMILES: Cc1nc(C)c(S(=O)(=O)Nc2ccccc2N2CCN(C(=O)OC(C)(C)C)CC2)s1] | ¹H NMR (CDCl₃) δ ppm 1.51 (s, 9 H) 2.53 (s, 3 H) 2.64 (s, 3 H) 2.64-2.69 (m, 4 H) 3.57 (br s, 4 H) 7.03-7.24 (m, 3 H) 7.57-7.68 (m, 1 H) 8.20 (NH) |
| 57 | 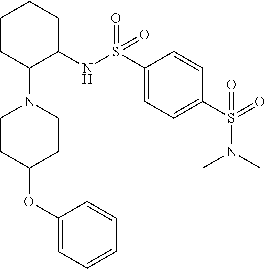 [SMILES: CN(C)S(=O)(=O)c1ccc(S(=O)(=O)NC2CCCCC2N2CCC(Oc3ccccc3)CC2)cc1] | ¹H NMR (CDCl₃) δ ppm 1.12-1.31 (m, 4 H) 1.53-1.93 (m, 6 H) 2.12 (br t, J = 8.16 Hz, 1 H) 2.21-2.30 (m, 2 H) 2.37 (br d, J = 10.63 Hz, 2 H) 2.48- 2.57 (m, 1 H) 2.71 (s, 6 H) 2.74-2.84 (m, 2 H) 4.24 (m, 1 H) 6.86 (br d, J = 8.06 Hz, 2 H) 6.93 (t, J = 7.33 Hz, 1 H) 7.26 (t, J = 7.93 Hz, 2 H) 7.91 (d, J = 8.34 Hz, 2 H) 8.07 (d, J = 8.34 Hz, 2 H) |
| 58 | 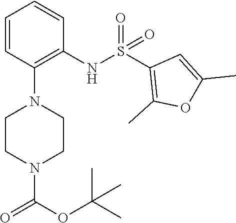 [SMILES: Cc1cc(S(=O)(=O)Nc2ccccc2N2CCN(C(=O)OC(C)(C)C)CC2)c(C)o1] | ¹H NMR (CDCl₃) δ ppm 1.51 (s, 9 H) 2.20 (s, 3 H) 2.41 (s, 3 H) 2.66 (t, J = 4.94 Hz, 4 H) 3.57 (br s, 4 H) 6.08 (d, J = 0.88 Hz, 1 H) 7.03-7.20 (m, 3 H) 7.52-7.62 (m, 1 H) 7.92 (NH) |
| 59 | 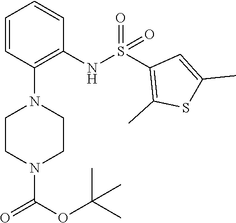 [SMILES: Cc1cc(S(=O)(=O)Nc2ccccc2N2CCN(C(=O)OC(C)(C)C)CC2)c(C)s1] | ¹H NMR (CDCl₃) δ ppm 1.51 (s, 9 H) 2.35 (s, 3 H) 2.49 (s, 3 H) 2.59 (br s, 4 H) 3.54 (br s, 4 H) 6.88 (d, J = 1.13 Hz, 1 H) 7.05-7.22 (m, 3 H) 7.59 (dd, J = 7.94, 1.31 Hz, 1 H) 7.89 (NH) |
| 60 | 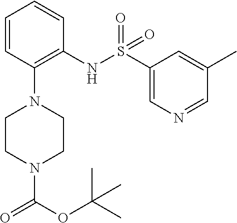 [SMILES: Cc1cncc(S(=O)(=O)Nc2ccccc2N2CCN(C(=O)OC(C)(C)C)CC2)c1] | ¹H NMR (CDCl₃) δ ppm 1.50 (s, 9 H) 2.39 (s, 3 H) 2.54 (t, J = 4.94 Hz, 4 H) 3.53 (br s, 4 H) 7.02-7.15 (m, 2 H) 7.15-7.23 (m, 1 H) 7.59 (d, J = 7.75 Hz, 1 H) 7.92 (s, 1 H) 8.06 (NH) 8.59 (s, 1 H) 8.83 (d, J = 1.88 Hz, 1 H) |
| 61 | 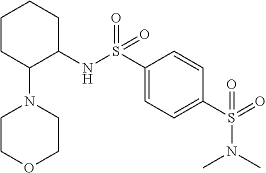 [SMILES: CN(C)S(=O)(=O)c1ccc(S(=O)(=O)NC2CCCCC2N2CCOCC2)cc1] | ¹H NMR (CDCl₃) δ ppm 1.02-1.24 (m, 2 H) 1.64 (m, 1 H) 1.73-1.88 (br s, 2 H) 2.06-2.34 (m, 3 H) 2.73 (s, 6 H) 3.18 (m, 3 H), 3.44-3.61 (br s, 4 H) 3.75 (m, 3 H) 7.89 (d, J = 8.25 Hz, 2 H) 8.04 (d, J = 8.25 Hz, 2 H) |
| 62 | 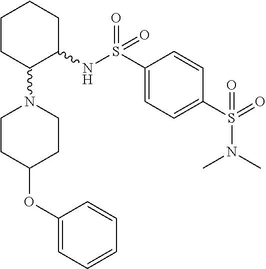 | ¹H NMR (CDCl₃) δ ppm 1.12-1.31 (m, 4 H) 1.53-1.93 (m, 6 H) 2.12 (br t, J = 8.16 Hz, 1 H) 2.21-2.30 (m, 2 H) 2.37 (br d, J = 10.63 Hz, 2 H) 2.48-2.57 (m, 1 H) 2.71 (s, 6 H) 2.74-2.84 (m, 2 H) 4.24 (m, 1 H) 6.86 (br d, J = 8.06 Hz, 2 H) 6.93 (t, J = 7.33 Hz, 1 H) 7.26 (t, J = 7.93 Hz, 2 H) 7.91 (d, J = 8.34 Hz, 2 H) 8.07 (d, J = 8.34 Hz, 2 H) |
| 63 | 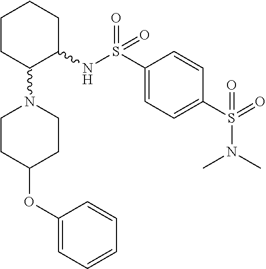 | ¹H NMR (CDCl₃) δ ppm 1.12-1.31 (m, 4 H) 1.53-1.93 (m, 6 H) 2.12 (br t, J = 8.16 Hz, 1 H) 2.21-2.30 (m, 2 H) 2.37 (br d, J = 10.63 Hz, 2 H) 2.48-2.57 (m, 1 H) 2.71 (s, 6 H) 2.74-2.84 (m, 2 H) 4.24 (m, 1 H) 6.86 (br d, J = 8.06 Hz, 2 H) 6.93 (t, J = 7.33 Hz, 1 H) 7.26 (t, J = 7.93 Hz, 2 H) 7.91 (d, J = 8.34 Hz, 2 H) 8.07 (d, J = 8.34 Hz, 2 H) |
| 64 | 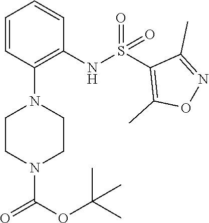 [SMILES: Cc1noc(C)c1S(=O)(=O)Nc1ccccc1N1CCN(C(=O)OC(C)(C)C)CC1] | ¹H NMR (CDCl₃) δ ppm 1.51 (s, 9 H) 2.34-2.40 (s, 3 H) 2.60 (s, 3 H) 2.71 (br t, J = 4.75 Hz, 4 H) 3.58 (br s, 4 H) 7.10-7.22 (m, 3 H) 7.41-7.51 (m, 1 H) 8.05 (NH) |
| 65 | 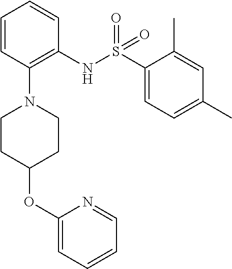 [SMILES: Cc1ccc(S(=O)(=O)Nc2ccccc2N2CCC(Oc3ccccn3)CC2)c(C)c1] | ¹H NMR (CDCl₃) δ ppm 1.92-2.05 (m, 2 H) 2.14-2.25 (m, 2 H) 2.34 (s, 3 H) 2.64 (s, 3 H) 2.73-2.84 (m, 2 H) 2.92-3.04 (m, 2 H) 5.26 (tt, J = 7.85, 3.85 Hz, 1 H) 6.78 (dt, J = 8.35, 0.77 Hz, 1 H) 6.89 (ddd, 5.07, 1.00 Hz, 1H) 6.95-7.12 (m, 4H) 7.18 (dd, J = 7.69, 1.69 Hz, 1 H) 7.40 (dd, J = 7.94, 1.56 Hz, 1 H) 7.61 (ddd, J = 8.35, 7.10, 2.06 Hz, 1 H) 7.95 (d, J = 8.13 Hz, 1 H) 8.15-8.19 (m, 1 H) 8.23 (NH) |
| 66 | 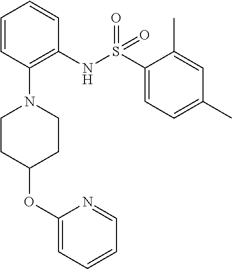 [SMILES: Cc1ccc(S(=O)(=O)Nc2ccccc2N2CCC(Oc3ccccn3)CC2)c(C)c1] | ¹H NMR (CDCl₃) δ ppm 0.98 (t, J = 7.44 Hz, 3 H) 1.60-1.70 (m, 2 H) 1.72-1.84 (m, 2 H) 2.03 (br dd, J = 12.51, 2.63 Hz, 2 H) 2.34 (s, 3 H) 2.62 (s, 3 H) 2.63-2.70 (m, 2 H) 2.83- 2.97 (m, 2 H) 3.42-3.55 (m, 3 H) 6.93- 7.04 (m, 2 H) 7.05 (s, 1 H) 7.09 (d, J = 8.25 Hz, 1 H) 7.13 (dd, J = 7.69, 1.56 Hz, 1 H) 7.38 (dd, J = 7.94, 1.56 Hz, 1 H) 7.94 (d, J = 8.13 Hz, 1 H) 8.19 (NH) |
| 67 | 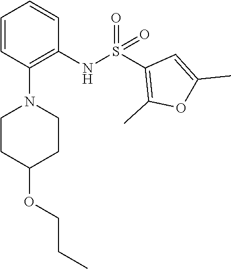 [SMILES: CCCOC1CCN(c2ccccc2NS(=O)(=O)c2cc(C)oc2C)CC1] | ¹H NMR (CDCl₃) δ ppm 0.98 (t, J = 7.38 Hz, 3 H) 1.60-1.67 (m, 2 H) 1.71-1.83 (m, 2 H) 1.99 (ddd, J = 9.32, 6.13, 3.06 Hz, 2 H) 2.18 (s, 3 H) 2.37-2.45 (m, 3 H) 2.52-2.69 (m, 2 H) 2.76-2.90 (m, 2 H) 3.39-3.53 (m, 3 H) 6.08 (d, J = 0.88 Hz, 1 H) 7.03 -7.15 (m, 2 H) 7.15-7.20 (m, 1 H) 7.57 (dd, J = 8.00, 1.50 Hz, 1 H) 8.01 (NH) |
| 68 | 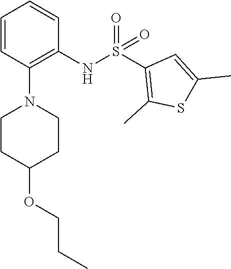 [SMILES: CCCOC1CCN(c2ccccc2NS(=O)(=O)c2cc(C)sc2C)CC1] | ¹H NMR (CDCl₃) δ ppm 0.97 (t, J = 7.44 Hz, 3 H) 1.59-1.68 (m, 2 H) 1.69-1.81 (m, 2 H) 1.94-2.04 (m, 2 H) 2.33 (s, 3 H) 2.50 (s, 3 H) 2.54 (br t, J = 9.13 Hz, 2 H) 2.72-2.81 (m, 2 H) 3.41-3.51 (m, 3 H) 6.89 (d, J = 1.13 Hz, 1 H) 7.03-7.14 (m, 2 H) 7.14-7.19 (m, 1 H) 7.58 (dd, J = 7.94, 1.56 Hz, 1 H) 7.89-8.21 (NH) |
| 70 | 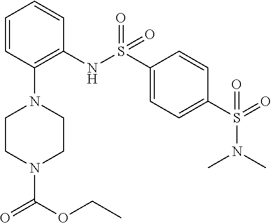 [SMILES: CCOC(=O)N1CCN(c2ccccc2NS(=O)(=O)c2ccc(S(=O)(=O)N(C)C)cc2)CC1] | ¹H NMR (CDCl₃) δ ppm 1.29 (t, J = 7.07, 3 H) 2.53 (m, 4 H) 2.73 (s, 6 H) 3.57 (m, 4 H) 4.18 (q,Hz, 2 H) 7.04-7.14 (m, 2 H) 7.14-7.21 (m, 1 H) 7.58 (d, J = 7.88 Hz, 1 H) 7.85 (d, J = 8.50, 2 H) 8.00 (d, J = 8.50 Hz, 2 H) 8.08 (NH) |
| 71 | 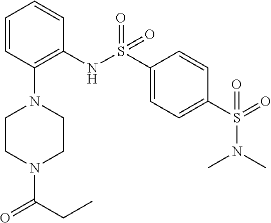 [SMILES: CCC(=O)N1CCN(c2ccccc2NS(=O)(=O)c2ccc(S(=O)(=O)N(C)C)cc2)CC1] | ¹H NMR (CDCl₃) δ ppm 1.10 (t, J = 7.44 Hz, 3 H) 2.30 (q, J = 7.44 Hz, 2 H) 2.48 (m, 4 H) 2.65 (s, 6 H) 3.48 (m, 2 H) 3.64 (m, 2 H) 7.00-7.05 (m, 2 H) 7.07-7.13 (m, 1 H) 7.49 (d, J = 8.00 Hz, 1 H) 7.75-7.81 (m, 2 H) 7.90-7.95 (m, 2 H) 7.98 (NH) |
| 72 | 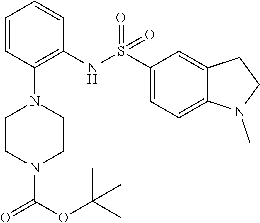 [SMILES: CN1CCc2cc(S(=O)(=O)Nc3ccccc3N3CCN(C(=O)OC(C)(C)C)CC3)ccc21] | ¹H NMR (CDCl₃) δ ppm 1.50 (s, 9 H) 2.36-2.56 (m, 4 H) 2.71 (s, 3 H) 2.94 (t, J = 8.38 Hz, 2 H) 3.37 (t, J = 8.32 Hz, 2 H) 3.48 (br s, 4 H) 6.72 (d, J = 1.50 Hz, 1 H) 7.00-7.10 (m, 4 H) 7.14- 7.20 (m, 1 H) 7.66-7.71 (m, 1 H) 7.85 (NH) |
| 73 | 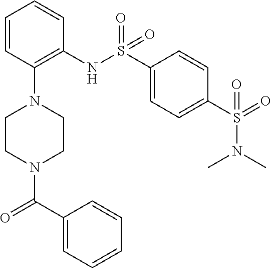 [SMILES: CN(C)S(=O)(=O)c1ccc(S(=O)(=O)Nc2ccccc2N2CCN(C(=O)c3ccccc3)CC2)cc1] | ¹H NMR (CDCl₃) δ ppm 2.62 (m, 4 H) 2.71 (s, 6 H) 3.48-3.99 (m, 4 H) 7.07-7.20 (m, 3 H) 7.44 (m, 5 H) 7.56 (d, J = 8.00 Hz, 1 H) 7.85 (d, J = 8.38 Hz, 2 H) 8.01 (d, J = 8.38 Hz, 2 H) 8.09 (NH) |
| 74 | 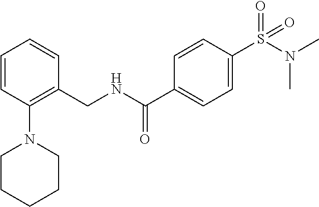 [SMILES: CN(C)S(=O)(=O)c1ccc(C(=O)NCc2ccccc2N2CCCCC2)cc1] | ¹H NMR (CDCl₃) δ ppm 1.58 (m, 2 H) 1.65-1.80 (m, 4 H) 2.70 (s, 6 H) 2.74-2.79 (m, 4 H) 4.32 (s, 2 H) 6.88 -6.98 (m, 1 H) 7.00 (d, J = 7.63 Hz, 2 H) 7.12-7.22 (m, 1 H) 7.32 (NH) 7.69-7.77 (m, 2 H) 7.84-7.91 (m, 2 H) |
| 75 | 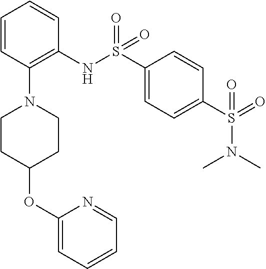 [SMILES: CN(C)S(=O)(=O)c1ccc(S(=O)(=O)Nc2ccccc2N2CCC(Oc3ccccn3)CC2)cc1] | ¹H NMR (CDCl₃) δ ppm 1.90 (m, 2 H) 2.07-2.16 (m, 2 H) 2.53-2.63 (m, 2 H) 2.69 (br s, 8 H) 5.21 (dt, J = 7.66, 3.86 Hz, 1 H) 6.75 (d, J = 8.25 Hz, 1 H) 6.85-6.90 (m, 1 H) 7.06-7.21 (m, 3 H) 7.56-7.67 (m, 2 H) 7.83 (d, J = 8.50 Hz, 2 H) 7.99 (d, J = 8.50 Hz, 2 H) 8.14 (dd, J = 5.07, 1.31 Hz, 1 H) |
| 76 | 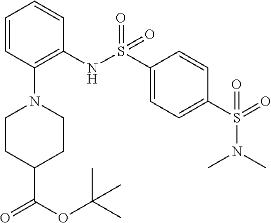 [SMILES: CN(C)S(=O)(=O)c1ccc(S(=O)(=O)Nc2ccccc2N2CCC(C(=O)OC(C)(C)C)CC2)cc1] | ¹H NMR (CDCl₃) δ ppm 1.49 (s, 9 H) 1.71-1.83 (m, 2 H) 1.94 (br dd, J = 13.07, 2.94 Hz, 2 H) 2.31 (tt, J = 10.90, 3.99 Hz, 1 H) 2.52 (dd, J = 7.57, 2.56 Hz, 4 H) 2.71 (s, 6 H) 7.03-7.16 (m, 3 H) 7.58-7.62 (m, 1 H) 7.83 (d, J = 8.50 Hz, 2 H) 7.98 (d, J = 8.50 Hz, 2 H) 8.09 (NH) |
| 77 | 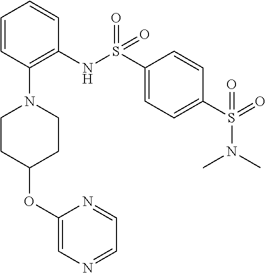 [SMILES: CN(C)S(=O)(=O)c1ccc(S(=O)(=O)Nc2ccccc2N2CCC(Oc3cnccn3)CC2)cc1] | ¹H NMR (CDCl₃) δ ppm 1.85-1.96 (m, 2 H) 2.06-2.16 (m, 2 H) 2.51- 2.61 (m, 2 H) 2.70 (br s, 8 H) 5.19 (dt, 3.71 Hz, 1 H) 7.06-7.20 (m, 3 H) 7.61 (dd, J = 7.82, 1.19 Hz, 1 H) 7.83 (d, J = 8.50 Hz, 2 H) 7.99 (d, J = 8.50 Hz, 2 H) 8.06 (dd, J = 2.63, 1.38 Hz, 1 H) 8.12 (d, J = 2.75 Hz, 1 H) 8.24 (d, J = 0.88 Hz, 1 H) |
| 78 | 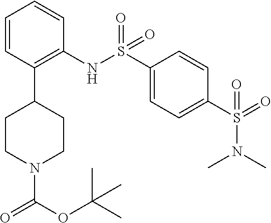 [SMILES: CN(C)S(=O)(=O)c1ccc(S(=O)(=O)Nc2ccccc2C2CCN(C(=O)OC(C)(C)C)CC2)cc1] | ¹H NMR (CDCl₃) δ ppm 1.47 (br s, 13 H) 2.70 (br s, 2 H) 2.75 (s, 6 H) 2.87-2.98 (m, 1 H) 4.16 (br s, 2 H) 6.96 (br d, J = 7.88 Hz, 1 H) 7.11 (br t, J = 7.19 Hz, 1 H) 7.19-7.31 (m, 2 H) 7.84-7.94 (m, 4 H) |
| 79 | 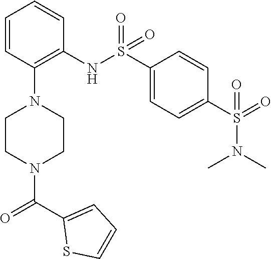 [SMILES: CN(C)S(=O)(=O)c1ccc(S(=O)(=O)Nc2ccccc2N2CCN(C(=O)c3cccs3)CC2)cc1] | ¹H NMR (CDCl₃) δ ppm 2.64 (br t, J = 4.57 Hz, 4 H) 2.71 (s, 6 H) 3.85 (m, 4 H) 7.04-7.22 (m, 4 H) 7.32 (d, J = 3.50 Hz, 1 H) 7.48 (d, J = 5.00 Hz, 1 H) 7.55 (d, J = 7.88 Hz, 1 H) 7.85 (d, J = 8.38 Hz, 2 H) 8.01 (br d, J = 8.38 Hz, 2 H) 8.08 (NH) |
| 80 | 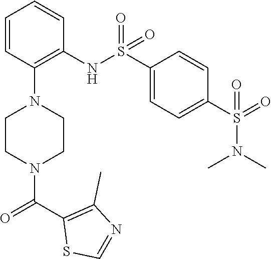 [SMILES: Cc1ncsc1C(=O)N1CCN(c2ccccc2NS(=O)(=O)c2ccc(S(=O)(=O)N(C)C)cc2)CC1] | ¹H NMR (CDCl₃) δ ppm 2.53 (s, 3 H) 2.65 (br t, J = 4.63 Hz, 4 H) 2.72 (s, 6 H) 3.73 (m, 4 H) 7.06-7.20 (m, 4 H) 7.53 (d, J = 7.88 Hz, 1 H) 7.85 (d, J = 8.38 Hz, 2 H) 8.01 (d, J = 8.38 Hz, 3 H) 8.78 (NH) |
| 81 | 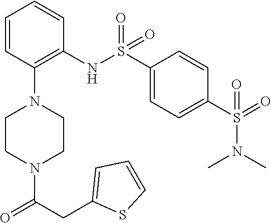 [SMILES: CN(C)S(=O)(=O)c1ccc(S(=O)(=O)Nc2ccccc2N2CCN(C(=O)Cc3cccs3)CC2)cc1] | ¹H NMR (CDCl₃) δ ppm 2.41-2.48 (m, 2 H) 2.56 (br d, J = 4.38 Hz, 2 H) 2.72 (s, 6 H) 3.59 (m, 2 H) 3.73 (m, 2 H) 3.95 (s, 2 H) 6.92 (d, J = 3.25 Hz, 1 H) 6.97 (dd, J = 5.00, 3.50 Hz, 1 H) 7.02-7.11 (m, 2 H) 7.15 (td, J = 7.66, 1.56 Hz, 1 H) 7.21-7.24 (m, 1 H) 7.53 (dd, J = 8.07, 1.06 Hz, 1 H) 7.84 (d, J = 8.38 Hz, 2 H) 7.99 (d, J = 8.38 Hz, 2 H) |
| 82 | 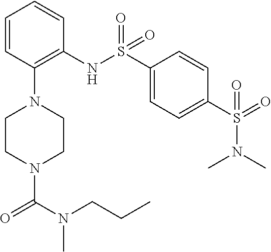 [SMILES: CCCN(C)C(=O)N1CCN(c2ccccc2NS(=O)(=O)c2ccc(S(=O)(=O)N(C)C)cc2)CC1] | ¹H NMR (CDCl₃) δ ppm 0.90 (t, J = 7.38 Hz, 3 H) 1.59 (dq, J = 14.76, 7.38 Hz, 2 H) 2.55 (br s, 4 H) 2.72 (s, 6 H) 2.85 (s, 3 H) 3.17 (br t, J = 7.38 Hz, 2 H) 3.29 (br s, 4 H) 7.04-7.19 (m, 3 H) 7.58 (br d, J = 8.00 Hz, 1 H) 7.83 (br d, J = 8.25 Hz, 2 H) 7.98 (br d, J = 8.38 Hz, 2 H) 8.08 (NH) |
| 83 | 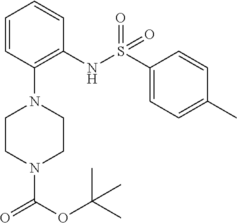 [SMILES: Cc1ccc(S(=O)(=O)Nc2ccccc2N2CCN(C(=O)OC(C)(C)C)CC2)cc1] | ¹H NMR (CDCl₃) δ ppm 1.48 (s, 9 H) 2.35 (s, 3 H) 2.38-2.51 (m, 4 H) 3.46 (br s, 4 H) 7.02-7.06 (m, 2 H) 7.09- 7.16 (m, 1 H) 7.21 (d, J = 8.13 Hz, 2 H) 7.59-7.63 (m, 1 H) 7.67 (d, J = 8.13 Hz, 2 H) 7.89 (N H) |
| 84 | 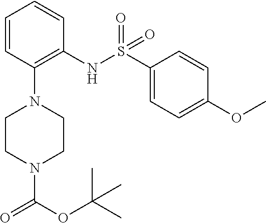 [SMILES: COc1ccc(S(=O)(=O)Nc2ccccc2N2CCN(C(=O)OC(C)(C)C)CC2)cc1] | ¹H NMR (CDCl₃) δ ppm 1.48 (s, 9 H) 2.45 (br s, 4 H) 3.47 (br s, 4 H) 3.80 (s, 3 H) 6.84-6.90 (m, 2 H) 7.00- 7.07 (m, 2 H) 7.10-7.16 (m, 1 H) 7.59-7.63 (m, 1 H) 7.69-7.75 (m, 2 H) 7.88 (NH) |
| 85 | 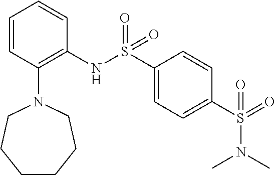 [SMILES: CN(C)S(=O)(=O)c1ccc(S(=O)(=O)Nc2ccccc2N2CCCCCC2)cc1] | ¹H NMR (CDCl₃) δ ppm 1.62-1.76 (br s, 8 H) 2.65-2.73 (br s, 10 H) 6.99 -7.13 (m, 3 H) 7.59 (d, J = 7.75 Hz, 1 H) 7.81 (d, J = 8.38 Hz, 2 H) 7.97 (d, J = 8.38 Hz, 2 H) 8.31 (NH) |
| 86 | 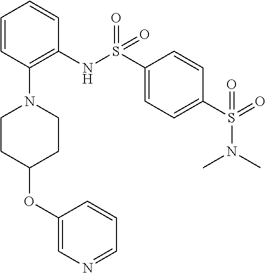 [SMILES: CN(C)S(=O)(=O)c1ccc(S(=O)(=O)Nc2ccccc2N2CCC(Oc3cccnc3)CC2)cc1] | ¹H NMR (CDCl₃) δ ppm 1.86 (m, 2 H) 1.98 (m, m2 H) 2.43 (m, 2 H) 2.62 (s, 6 H) 2.70 (m, 2 H) 4.42 (br s, 1 H) 6.98-7.12 (m, 3 H) 7.16 (m, 2 H) 7.52 (br d, J = 7.88 Hz, 1 H) 7.75 (br d, J = 8.25 Hz, 2 H) 7.91 (br d, J = 8.25 Hz, 2 H) 8.04 (NH) 8.17 (m, 1 H) 8.27 (m, 1 H) |
| 87 | 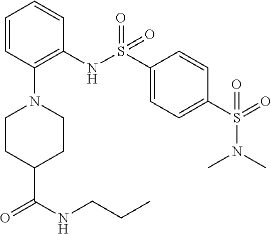 [SMILES: CCCNC(=O)C1CCN(c2ccccc2NS(=O)(=O)c2ccc(S(=O)(=O)N(C)C)cc2)CC1] | ¹H NMR (CD₃OD) δ ppm 0.95 (t, J = 7.44 Hz, 3 H) 1.55 (sxt, J = 7.25 Hz, 2 H) 1.67-1.78 (m, 2 H) 1.84 (qd, J = 12.15, 3.81 Hz, 2 H) 2.27 (tt, J = 11.57, 3.94 Hz, IH) 2.39-2.47 (m, 2 H) 2.49-2.61 (m, 2 H) 2.68 (s, 6 H) 3.17 (t,Hz, 2 H) 7.08-7.21 (m, 3 H) 7.58 (dd, J = 7.57, 1.56 Hz, 1 H) 7.90 (d, J = 8.50 Hz, 2 H) 8.03 (d, J = 8.50 Hz, 2 H) |
| 88 | 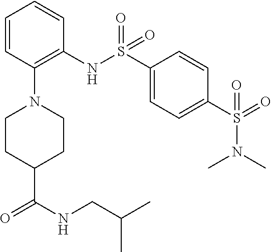 [SMILES: CC(C)CNC(=O)C1CCN(c2ccccc2NS(=O)(=O)c2ccc(S(=O)(=O)N(C)C)cc2)CC1] | ¹H NMR (CD₃OD) δ ppm 0.92 (d, J = 6.63 Hz, 6 H) 1.66-1.76 (m, 2 H) 1.77-1.90 (br s, 3 H) 2.22-2.34 (m, 1 H) 2.42 (br d, J = 11.38 Hz, 2 H) 2.50 -2.60 (m, 2 H) 2.66 (s, 6 H) 3.02 (br d, J = 6.88 Hz, 2 H) 7.06-7.19 (m, 3 H) 7.52-7.60 (m, 1 H) 7.88 (br d, J = 8.25 Hz, 2 H) 8.01 (br d, J = 8.38 Hz, 2 H) |
| 89 | 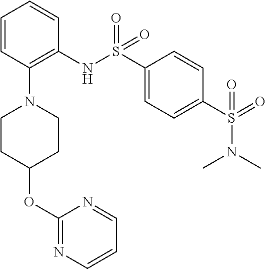 [SMILES: CN(C)S(=O)(=O)c1ccc(S(=O)(=O)Nc2ccccc2N2CCC(Oc3ncccn3)CC2)cc1] | ¹H NMR (CDCl₃) δ ppm 1.97 (br s, 2 H) 2.07 (br d, J = 3.50 Hz, 2 H) 2.50 (br s, 2 H) 2.67 (s, 6 H) 2.76 (br s, 2 H) 5.18 (br s, 1 H) 6.94 (t, J = 4.75 Hz, 1 H) 7.03-7.18 (br s, 3 H) 7.56-7.64 (m, 1 H) 7.80 (d, J = 8.25 Hz, 2 H) 7.97 (d, J = 8.50 Hz, 2 H) 8.14 (NH) 8.48- 8.54 (m, 2 H) |
| 90 | 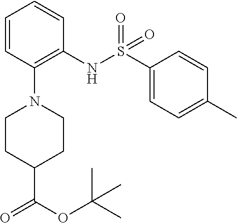 [SMILES: Cc1ccc(S(=O)(=O)Nc2ccccc2N2CCC(C(=O)OC(C)(C)C)CC2)cc1] | ¹H NMR (CDCl₃) δ ppm 1.48 (s, 9 H) 1.71-1.82 (m, 2 H) 1.91 (br dd, J = 13.26, 3.25 Hz, 2 H) 2.28 (tt, J = 10.90, 4.11 Hz, 1 H) 2.35 (s, 3 H) 2.44-2.56 (m, 4 H) 6.97-7.07 (m, 2 H) 7.07-7.13 (m, 1 H) 7.20 (d, J = 8.25 Hz, 2 H) 7.61 (dd, J = 8.13, 1.25 Hz, 1 H) 7.69 (d, J = 8.25 Hz, 2 H) 7.94 (NH) |
| 91 | 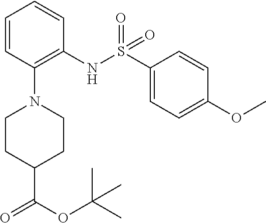 [SMILES: COc1ccc(S(=O)(=O)Nc2ccccc2N2CCC(C(=O)OC(C)(C)C)CC2)cc1] | ¹H NMR (CDCl₃) δ ppm 1.48 (s, 9 H) 1.71-1.82 (m, 2 H) 1.92 (br dd, J = 13.26, 3.13 Hz, 2 H) 2.28 (tt, J = 10.87, 4.08 Hz, 1 H) 2.44-2.58 (m, 4 H) 3.80 (s, 3 H) 6.85-6.90 (m, 2 H) 6.97-7.07 (m, 2 H) 7.07-7.13 (m, 1 H) 7.60 (dd, J = 8.00, 1.00 Hz, 1 H) 7.74 (d, J = 8.88 Hz, 2 H) 7.92 (NH) |
| 92 | 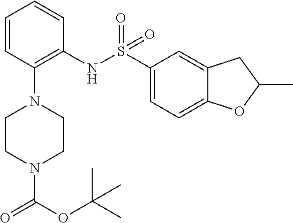 [SMILES: CC1Cc2cc(S(=O)(=O)Nc3ccccc3N3CCN(C(=O)OC(C)(C)C)CC3)ccc2O1] | ¹H NMR (CDCl₃) δ ppm 1.47 (d, J = 6.25 Hz, 3 H) 1.49-1.53 (m, 9 H) 2.51 (br s, 4 H) 2.80 (dd, J = 15.82, 7.44 Hz, 1 H) 3.31 (dd, J = 15.82, 8.94 Hz, 1 H) 3.51 (br s, 4 H) 4.93-5.09 (m, 1 H) 6.72 (d, J = 8.50 Hz, 1 H) 6.92- 7.12 (m, 2 H) 7.16 (ddd, J = 8.29, 6.47, 2.25 Hz, 1 H) 7.51-7.67 (m, 3 H) 7.86 (NH) |
| 93 | 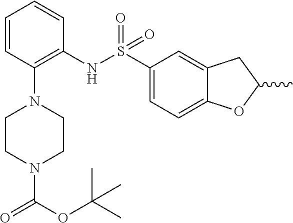 [SMILES: CC1Cc2cc(S(=O)(=O)Nc3ccccc3N3CCN(C(=O)OC(C)(C)C)CC3)ccc2O1] | ¹H NMR (CDCl₃) δ ppm 1.37 (d, J = 6.25 Hz, 3 H) 1.41 (s, 9 H) 2.42 (br s, 4 H) 2.70 (dd, J = 15.82, 7.44 Hz, 1 H) 3.22 (dd, J = 15.82, 8.94 Hz, 1 H) 3.42 (br s, 4 H) 4.87-4.99 (m, 1 H) 6.63 (d, J = 8.38 Hz, 1 H) 6.95-7.02 (m, 2 H) 7.04-7.11 (m, 1 H) 7.47- 7.56 (m, 3 H) 7.77 (NH) |
| 94 | 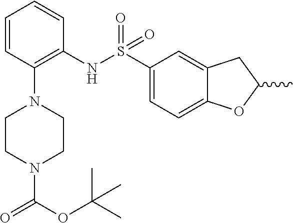 [SMILES: CC1Cc2cc(S(=O)(=O)Nc3ccccc3N3CCN(C(=O)OC(C)(C)C)CC3)ccc2O1] | ¹H NMR (CDCl₃) δ ppm 1.37 (d, J = 6.25 Hz, 3 H) 1.41 (s, 9 H) 2.42 (br s, 4 H) 2.70 (dd, J = 15.82, 7.44 Hz, 1 H) 3.22 (dd, J = 15.82, 8.94 Hz, 1 H) 3.42 (br s, 4 H) 4.87-4.99 (m, 1 H) 6.63 (d, J = 8.38 Hz, 1 H) 6.95-7.02 (m, 2 H) 7.04-7.11 (m, 1 H) 7.47- 7.56 (m, 3 H) 7.77 (NH) |
| 95 | 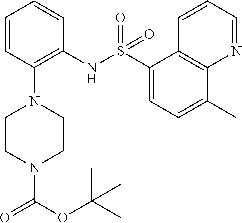 [SMILES: Cc1ccc(S(=O)(=O)Nc2ccccc2N2CCN(C(=O)OC(C)(C)C)CC2)c2cccnc12] | ¹H NMR (CDCl₃) δ ppm 1.51 (s, 9 H) 2.48 (br s, 4 H) 2.84 (s, 3 H) 3.48 (br s, 4 H) 6.95-7.03 (m, 2 H) 7.08 (td, J = 7.54, 1.94 Hz, 1 H) 7.49 (dd, J = 8.19, 1.06 Hz, 1 H) 7.57 (dd, J = 8.69, 4.19 Hz, 1 H) 7.62 (d, J = 7.50 Hz, 1 H) 8.24-8.29 (m, 1 H) 8.27- 8.29 (m, 1 H) 9.01 (dd, J = 4.13, 1.63 Hz, 1 H) 9.08 (dd, J = 8.63, 1.63 Hz, 1 H) |
| 96 | 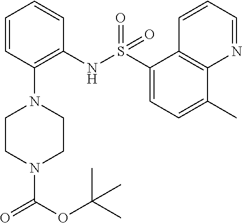 [SMILES: Cc1ccc(S(=O)(=O)Nc2ccccc2N2CCN(C(=O)OC(C)(C)C)CC2)c2cccnc12] | ¹H NMR (CDCl₃) δ ppm 1.08 (br d, J = 7.00 Hz, 6 H) 1.51 (s, 9 H) 2.10 (br s, 2 H) 2.75 (br s, 2 H) 2.89 (s, 3 H) 3.26-3.52 (m, 4 H) 4.75 (br s, 1 H) 6.71 (br s, 1 H) 7.00-7.17 (m, 3 H) 7.44-7.66 (m, 4 H) |
| 97 | 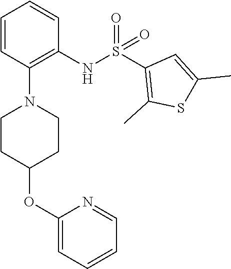 [SMILES: Cc1cc(S(=O)(=O)Nc2ccccc2N2CCC(Oc3ccccn3)CC2)c(C)s1] | ¹H NMR (CDCl₃) δ ppm 1.77-1.92 (m, 2 H) 2.00-2.12 (m, 2 H) 2.23 (s, 3 H) 2.38-2.47 (m, 3 H) 2.52-2.67 (m, 2 H) 2.69-2.79 (m, 2 H) 5.15 (tt, J = 7.83, 3.86 Hz, 1 H) 6.68 (d, J = 8.38 Hz, 1 H) 6.76-6.82 (m, 2 H) 6.96- 7.07 (m, 2 H) 7.11 (dd, J = 7.63, 1.63 Hz, 1 H) 7.48-7.55 (m, 2 H) 7.94 (NH) 8.05-8.13 (m, 1 H) |
| 98 | 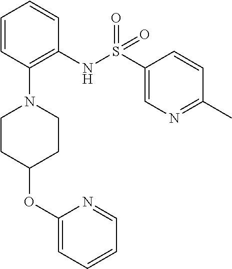 [SMILES: Cc1ccc(S(=O)(=O)Nc2ccccc2N2CCC(Oc3ccccn3)CC2)cn1] | ¹H NMR (CDCl₃) δ ppm 1.87-2.02 (m, 2 H) 2.08-2.18 (m, 2 H) 2.59 (s, 3 H) 2.63 (br d, J = 8.50 Hz, 2 H) 2.74 (br d, J = 4.50 Hz, 2 H) 5.23 (tt, J = 7.72, 3.78 Hz, 1 H) 6.77 (d, J = 8.38 Hz, 1 H) 6.88 (ddd, 5.07, 1.00 Hz, 1 H) 7.05-7.12 (m, 1 H) 7.16 (qd, J = 7.50, 1.50 Hz, 2 H) 7.22 (d, J = 8.13 Hz, 1 H) 7.56-7.66 (m, 2 H) 7.99 (dd, J = 8.25, 2.38 Hz, 1 H) 8.16 (ddd, J = 5.03, 1.97, 0.75 Hz, 2 H) 8.92 (d, J = 2.13 Hz, 1 H) |
| 99 | 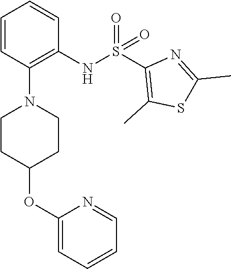 [SMILES: Cc1nc(S(=O)(=O)Nc2ccccc2N2CCC(Oc3ccccn3)CC2)c(C)s1] | ¹H NMR (CDCl₃) δ ppm 1.98-2.10 (m, 2 H) 2.14-2.26 (m, 2 H) 2.55 (s, 3 H) 2.67 (s, 3 H) 2.71-2.81 (m, 2 H) 2.97 (tt, J = 7.41, 3.47 Hz, 2 H) 5.27 (tt, J = 7.66, 3.85 Hz, 1 H) 6.78 (dt, J = 8.38, 0.81 Hz, 1 H) 6.88 (ddd, 5.07, 0.88 Hz, 1 H) 7.03- 7.14 (m, 2 H) 7.22 (dd, J = 7.69, 1.69 Hz, 1 H) 7.57-7.65 (m, 2 H) 8.14- 8.20 (m, 1 H) 8.52 (NH) |
| 100 | 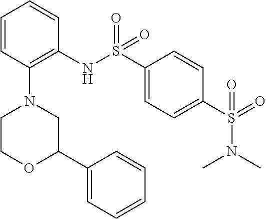 [SMILES: CN(C)S(=O)(=O)c1ccc(S(=O)(=O)Nc2ccccc2N2CCOC(c3ccccc3)C2)cc1] | ¹H NMR (CDCl₃) δ ppm 2.33 (br d, J = 11.63 Hz, 1 H) 2.48-2.53 (m, 1 H) 2.59 (s, 6 H) 2.61-2.70 (m, 1 H) 2.83 (td, J = 11.57, 3.25 Hz, 1 H) 3.83 (td, J = 11.44, 2.38 Hz, IH) 4.01-4.07 (m, 1 H) 4.55 (dd, J = 10.13, 2.38 Hz, 1 H) 6.98-7.13 (m, 3 H) 7.23-7.32 (m, 5 H) 7.52 (dd, J = 7.94, 1.31 Hz, 1 H) 7.71-7.77 (m, 2 H) 7.91-7.97 (m, 2 H) 8.06 (NH) |
| 101 | 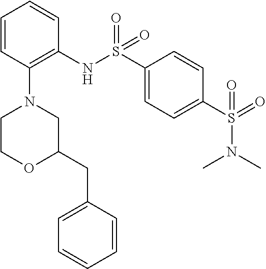 [SMILES: CN(C)S(=O)(=O)c1ccc(S(=O)(=O)Nc2ccccc2N2CCOC(Cc3ccccc3)C2)cc1] | ¹H NMR (CDCl₃) δ ppm 2.30 (br d, J = 11.38 Hz, 1 H) 2.40-2.49 (m, 1 H) 2.57 (dd, J = 11.38, 9.88 Hz, 1 H) 2.68 (dd, J = 13.88, 6.63 Hz, 1 H) 2.73 (s, 6 H) 2.80 (td, J = 11.51, 3.13 Hz, 1 H) 2.95 (dd, J = 13.88, 6.63 Hz, 1 H) 3.74 (td, J = 11.41, 2.44 Hz, 1H) 3.81-3.91 (m, 1 H) 3.94-4.01 (m, 1 H) 7.06- 7.12 (m, 1 H) 7.12-7.18 (m, 2 H) 7.22-7.27 (m, 2 H) 7.29-7.32 (m, 1 H) 7.32-7.38 (m, 2 H) 7.50-7.56 (m, 1 H) 7.75-7.81 (m, 2 H) 7.89-7.94 (m, 2 H) 8.10 (NH) |
| 103 | 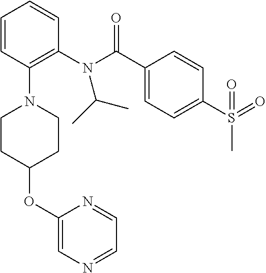 [SMILES: CC(C)N(C(=O)c1ccc(S(C)(=O)=O)cc1)c1ccccc1N1CCC(Oc2cnccn2)CC1] | ¹H NMR (CDCl₃) δ ppm 1.11 (br d, J = 7.00 Hz, 6H) 1.75 (br s, 1 H) 1.85- 2.11 (m, 4 H) 2.20 (br s, 1 H) 2.76 (br s, 1 H) 2.89 (br s, 3 H) 3.06 (m, 1 H) 4.25-4.25 (m, 1 H) 5.05 (br s, 1 H) 6.78 (br s, 1 H) 6.99-7.06 (m, 1 H) 7.10 (br dd, J = 7.63, 1.63 Hz, 1 H) 7.51 (br s, 2 H) 7.60 (br s, 2 H) 7.98 (dd, J = 2.75, 1.38 Hz, 1 H) 8.04 (d, J = 2.75 Hz, 1 H) 8.05-8.08 (m, 1 H) 8.15 (d, J = 1.25 Hz, 1 H) |
| 105 | 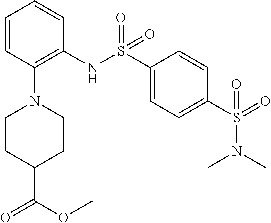 [SMILES: COC(=O)C1CCN(c2ccccc2NS(=O)(=O)c2ccc(S(=O)(=O)N(C)C)cc2)CC1] | ¹H NMR (CDCl₃) δ ppm 1.67-1.84 (m, 2 H) 1.89 (br dd, J = 13.20, 3.19 Hz, 2 H) 2.29-2.51 (m, 5 H) 2.58- 2.70 (m, 6 H) 3.65 (s, 3 H) 6.95- 7.11 (m, 3 H) 7.52 (dd, J = 8.00, 1.00 Hz, 1 H) 7.71-7.81 (m, 2 H) 7.86- 7.93 (m, 2 H) 7.98 (NH) |
| 106 | 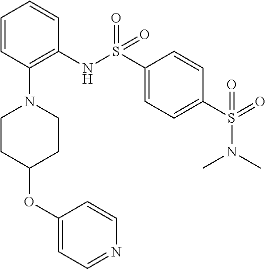 [SMILES: CN(C)S(=O)(=O)c1ccc(S(=O)(=O)Nc2ccccc2N2CCC(Oc3ccncc3)CC2)cc1] | ¹H NMR (CDCl₃) δ ppm 1.80-1.91 (m, 2 H) 1.98-2.06 (m, 2 H) 2.38- 2.55 (m, 2 H) 2.62 (s, 6 H) 2.64- 2.75 (m, 2 H) 4.42-4.55 (m, 1 H) 6.74 (br d, J = 5.50 Hz, 2 H) 6.98- 7.05 (m, 1 H) 7.05-7.14 (m, 2 H) 7.51 (br d, J = 7.88 Hz, 1 H) 7.75 (d, J = 8.38 Hz, 2 H) 7.91 (d, J = 8.38 Hz, 2 H) 8.36 (br d, J = 3.38 Hz, 2 H) |

Pharmacology

Cellular TRPML1 Assay

Buffers and reagents
PBS (D-PBS without calcium and magnesium; EuroClone)
Trypsin (Trypsin 0.05%, EDTA 0.02% in PBS; EuroClone)
DMSO (Sigma)
Ca²⁺ free Tyrode's buffer: in-house solution (130 mM NaCl, 5 mM KCl, 1 mM MgCl2, 5 mM NaHCO₃, 20 mM HEPES in water at pH 7.4; sterile filtered).
Opti-MEM (Gibco)
Agonist: ML-SA1 (Sigma); stock: 60 mM in DMSO, stored at −20° C.
Blocker: ML-SI3 (in house synthesis); stock: 20 mM in DMSO, stored at −20° C.

Cell Line

The final clone for the TRPML1 assay is HEK T-REx/GCaMP6f/TRPML1.

GCaMP6f is a genetically encoded calcium indicator that is stably expressed in this cell line and used as a fluorescent read-out.

Assay Protocol

Experiments are performed in 384 MTP format. Cells are seeded at 15000 cells/well either in 25 μl/well of growth medium or in 20 μl/well of Optimem+0.5% FBS without selection antibiotics. Twenty-four hours later, cells are assayed for the response to various compounds using the Ca²⁺ sensitive GCaMP6f protein stably expressed in the cells as readout.

The experiment is performed in a 384-well format according to the following procedures for either Ca²⁺ free or Optimem conditions:
Ca²⁺ free condition (in absence of extracellular Ca²⁺):
24 h after seeding, pre-incubate the cells at room temperature for about 10′.
Then remove the culture medium and replace it with 20 μL of Ca²⁺ free Tyrode's buffer.
Start the experiment at the FLIPR^{TETRA} by injecting 10 μL/w of 3× concentrated test compounds and controls in Ca²⁺ free Tyrode's buffer. Monitor the kinetic response over a period of 300 seconds.
Final DMSO concentration: 0.5%
Optimem condition (in presence of extracellular Ca²⁺):
24 h after seeding, pre-incubate the cells at room temperature for about 10′.
Start the experiment at the FLIPR^{TETRA} by injecting 10 μL/w of 3× concentrated test compounds and controls in Ca²⁺ free Tyrode's buffer. Monitor the kinetic response over a period of 300 seconds.
Final DMSO concentration: 0.5%

Data from FLIPR^{TETRA} measurements are analyzed with the Genedata Screener® software.

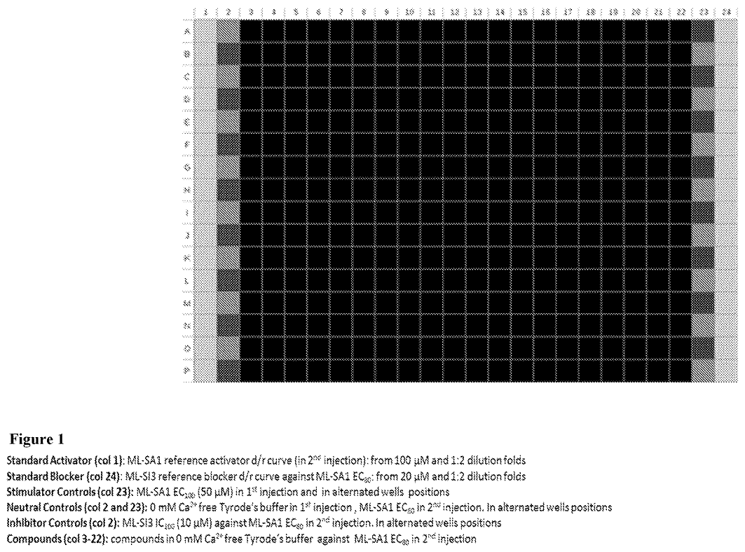

FIG. 1 shows an Example plate layout

Stimulator controls (Col 2): injection of ML-SA1 EC₁₀₀ (50 μM) in alternated wells positions

Neutral controls (Col 2): injection of 0 mM Ca²⁺ free Tyrode's buffer in alternated wells positions.

Test wells (Col 3-22): test compounds dose response curve from either 100 μM with 1:2 dilution steps or from 50 μM with 1:3.16 dilution steps.

Data Analysis

| | |
|---|---|
| Compound | Calculate the Kinetic Response Value (KRV) as: |
| % Activity | MAX_{CA}/Baseline = [max(sec5 . . . 290)/Mean (see 1 . . . sec2)] |
| | Normalize the KRV to the median (< >) of Neutral and Stimulator |
| | control wells: |
| | $Activity [ \% ] \_Ago = 100 * ( \frac{x - \text{<NeutralControls>}}{\text{<Stimulator Controls>} - \text{<NeutralControls>}} )$ |

TABLE 5
Activity of TRPML1 agonists

| Example | Ec₅₀ (μM) |
|---|---|
| 1 | 91.1 |
| 2 | 58.9 |
| 3 | 50.2 |
| 4 | 8.4 |
| 5 | 6.0 |
| 6 | 1.7 |
| 7 | 85.4 |
| 8 | 64.0 |
| 9 | 47.4 |
| 10 | 8.4 |
| 11 | 33.5 |
| 12 | 2.8 |
| 13 | 2.1 |
| 14 | 19.7 |
| 15 | 59.1 |
| 16 | 14.6 |
| 17 | 18.2 |
| 18 | 3.5 |
| 19 | 2.1 |
| 20 | 3.4 |
| 21 | 55.6 |
| 22 | 98.0 |
| 23 | 15.5 |
| 24 | 2.1 |
| 25 | 1.8 |
| 26 | 4.5 |
| 27 | 13.3 |
| 28 | 2.9 |
| 29 | 2.3 |
| 30 | 50.0 |
| 31 | 6.0 |
| 32 | 19.2 |
| 33 | 10.8 |
| 35 | 7.7 |
| 36 | 7.3 |
| 37 | 6.3 |
| 38 | 34.5 |
| 39 | 54.5 |
| 40 | 3.0 |
| 41 | 4.4 |
| 42 | 5.6 |
| 43 | 7.0 |
| 44 | 2.6 |
| 45 | 1.9 |
| 46 | 9.4 |
| 47 | 5.2 |
| 49 | 29.3 |
| 50 | 2.2 |
| 51 | 3.0 |
| 52 | 7.2 |
| 53 | 3.6 |
| 54 | 20.0 |
| 55 | 6.1 |
| 56 | 3.6 |
| 57 | 3.0 |
| 58 | 2.3 |
| 59 | 1.6 |
| 60 | 7.3 |
| 61 | 39.5 |
| 62 | 15.9 |
| 63 | 1.7 |
| 64 | 36.4 |
| 65 | 3.5 |
| 66 | 22.1 |
| 67 | 37.8 |
| 68 | 4.6 |
| 70 | 3.2 |
| 71 | 17.3 |
| 72 | 3.0 |
| 73 | 6.4 |
| 74 | 3.2 |
| 75 | 1.6 |
| 76 | 1.3 |
| 77 | 1.7 |
| 78 | 6.9 |
| 79 | 8.8 |
| 80 | 18.9 |
| 81 | 11.1 |
| 82 | 5.9 |
| 83 | 1.8 |
| 84 | 4.2 |
| 85 | 1.6 |
| 86 | 5.7 |
| 87 | 18.5 |
| 88 | 6.5 |
| 89 | 3.2 |
| 90 | 2.5 |
| 91 | 3.3 |
| 92 | 2.4 |
| 93 | 4.9 |
| 94 | 3.8 |
| 95 | 10.7 |
| 96 | 7.3 |
| 97 | 2.8 |
| 98 | 3.3 |
| 99 | 5.0 |
| 100 | 6.8 |
| 101 | 4.0 |
| 103 | 14.8 |
| 105 | 18.0 |
| 106 | 8.6 |

TRPML1—Automated Patch-Clamp

Cell Culture Conditions

HEK T-REx/GcaMP6f/TRPML1 cells were cultured in DMEM High Glucose (Lonza BioWhittaker cat. BE12-604F/U1; 500 mL) supplemented with Fetal Bovine Serum TET-FREE (Euroclone cat. EC S0182L; 50 mL, Penicillin-Streptomycin (BioWhittaker, cat. DE17-602E; 5 mL of 100× Solution), G418 (Sigma, cat. G8168; 0.25 mg/ml) and Zeocyn (InvivoGen, cat. ant-zn-1; 50 μg/ml).

Experimental Protocol

HEK T-REx/GcaMP6f/TRPML1 cells were seeded 48 or 72 hours before experiment, at a concentration of 8*10⁶/4*10⁶ cells onto a T225 flask. Just before the experiments, cells were washed twice with D-PBS w/o Ca²⁺/Mg²⁺ (Euroclone cat. ECB4004L) and detached from the flask with trypsin-EDTA (Sigma cat. T4174; diluted 1/10). Cells were then re-suspended in the suspension solution: 25 mL EX-CELL ACF CHO medium(Sigma cat. C5467); 0.625 mL HEPES (Sigma cat. H0887); 0.25 mL of 100× Penicillin/Streptomycin (Euroclone cat. ECB3001D), 0.1 mL of Soybean Trypsin Inhibitor 10 mg/mL (Sigma cat. T6522) and placed on the QPatch 16X.

Ligands and Buffer

Compounds:

2 (two) agonists solubilized 100% DMSO (20 mM) were tested in three concentrations (0.5, 5, 50 μM). Dilution from stock were prepared just before the experiments in the extracellular solution (0.25% final DMSO concentration). DMSO solution was obtained from AppliChem (cat. A3672)

Patch Clamp Solutions:
Intracellular solution (mM): 120 CsMeSO3, 4 NaCl, 2 MgCl2, 10 EGTA, 20 HEPES, 2 Mg-ATP (pH 7.2 with CsOH).
Extracellular solution (mM): 140 NaGluconate, 5 KCl, 1 MgCl2, 2 CaCl₂), 10 Glucose, 10 MES (pH 4.6 with HCl).

Patch Clamp Analysis—QPatch 16X

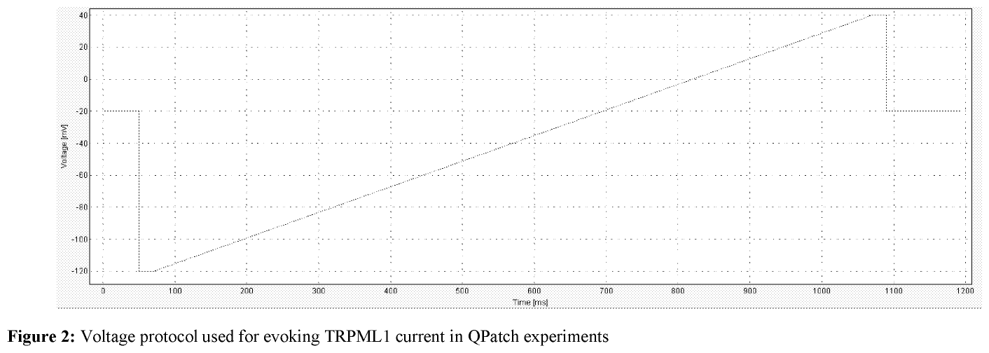

For the voltage clamp experiments on TRPML1, data were sampled at 5 KHz. After establishment of the seal and the passage in the whole cell configuration, the cells were held at −20 mV and the TRPML1 current was evoked using the voltage protocol illustrated in FIG. 2, applied every 5 sec. (50 ms at −20 mV; 20 ms at −120 mV, 1000 ms ramp from −120 to +20 mV; 20 ms at +40 mV, and back to −20 mV); in the absence (vehicle period, i.e. 0.25% DMSO) and in the presence of increasing concentrations (3) of the compound under investigation.

Data Analysis

Agonists: For data collection, the Sophion proprietary software was used and the analysis was performed off-line using Excel and GraphPad Prism (V 8.00). Results are reported in Table 6, expressed as TRPML1 current fold increase evoked by the compound under investigation (i.e. current in the presence of the compound/current in vehicle).

TABLE 6
QPatch activity of TRPML1 agonists

| | Qpatch Assay | |
|---|---|---|
| | TRPML1 agonism | |
| | Current fold increase | |
| Example | Mean ± SEM (n) | |
| 6 | 0.3 μM: | 1.0 ± 0.77 (3) |
| | 3.0 μM: | 32.7 ± 8.9 (3) |
| 13 | 0.3 μM: | 13.5 ± 4.8 (4) |
| | 3.0 μM: | 208 ± 15.7 (4) |
| | 30 μM: | 244 ± 27.6 (4) |
| 24 | 0.3 μM: | 4.6 ± 1.8 (4) |
| | 3 μM: | 168.8 ± 96.1 (4) |
| | 30 μM: | 687.7 (1) |
| 25 | 0.3 μM: | 3.5 ± 1.8 (3) |
| | 3 μM: | 177.1 ± 38.2 (3) |
| | 30 μM: | 1013.8 (1) |
| 26 | 0.3 μM: | 0.46 ± 0.16 (3) |
| | 3.0 μM: ) | 41.4 ± 21.1 (3 |
| | 30 μM: | 390 ± 158 (3) |
| 52 | 0.3 μM: | 1.3 ± 1.0 (3) |
| | 3 μM: | 10.4 ± 3.8 (3) |
| | 30 μM: | 472.5 ± 267.9 (3) |
| 53 | 0.3 μM: | 1.9 ± 1.6 (3) |
| | 3 μM: | 88.3 ± 59.9 (3) |
| | 30 μM: | 257.4 ± 190.7 (3) |
| 57 | 0.3 μM: | 0.39 ± 0.14 (2) |
| | 3.0 μM: | 0.86 ± 0.68 (2) |
| | 30 μM: | 4.3 ± 2.2 (2) |
| 63 | 0.3 μM: | 0.042 ± 0.153 (2) |
| | 3.0 μM: | 0.86 ± 0.60 (2) |
| | 30 μM: | 4.3 ± 0.9 (2) |
| 70 | 0.3 μM: | 0.5 ± 0.3 (3) |
| | 3 μM: | 22.5 ± 10.6 (3) |
| | 30 μM: | 656.7 ± 150.5 (3) |
| 73 | 0.3 μM: | −0.070 |
| | 3.0 μM: | −0.31 |
| | 30 μM: | 11.3 |
| 74 | 0.3 uM: | 4.3 ± 2.0 (6) |
| | 3.0 uM: | 356 ± 69.7 (6) |
| | 30 uM: | 639 + 241 (3) |
| 75 | 0.3 uM: | 310 ± 147 (4) |
| | 3.0 uM: | 435 ± 172 (4) |
| | 30 uM: | 603 ± 155 (3) |
| 76 | 0.3 uM: | 35.6 ± 26.7 (3) |
| | 3.0 uM: | 38.5 ± 24.8 (2) |
| | 30 uM: | 52.7 ± 36.3 (2) |
| 77 | 0.3 uM: | 74.4 ± 13.8 (3) |
| | 3.0 uM: | 167 ± 10.7 (3) |
| | 30 uM: | 168 ± 9.8 (3) |
| 78 | 0.3 uM: | 0.68 ± 0.34 (3) |
| | 3.0 uM: | 16.5 ± 9.1 (3) |
| | 30 uM: | 166 ± 75.7 (3) |
| 82 | 0.3 uM: | 2.5 ± 1.3 (3) |
| | 3.0 uM: | 12.5 ± 8.6 (3) |
| | 30 uM: | 138 ± 55.7 (3) |
| 83 | 0.3 uM: | 70.2 ± 13.8 (4) |
| | 3 uM: | 125.0 ± 31.0 (4) |
| | 30 uM: | 140.3 ± 36.8 (4) |
| 84 | 0.3 uM: | 8.6 ± 1.3 (2) |
| | 3.0 uM: | 32.9 ± 3.5 (2) |
| | 30 uM: | 42.7 ± 1.4 (2) |
| 85 | 0.3 uM: | 17.7 ± 5.5 (4) |
| | 3.0 uM: | 206 ± 167 (2) |
| | 30 uM: | 260 ± 202 (2) |
| 88 | 0.3 uM: | −0.066 ± 0.080 (3) |
| | 3.0 uM: | 3.9 ± 2.5 (3) |
| | 30 uM: | 125 ± 75.3 (3) |
| 90 | 0.3 uM: | 140 ± 44.1 (4) |
| | 3.0 uM: | 83.1 ± 34.0 (2) |
| | 30 uM: | 112 ± 38.1 (2) |
| 91 | 0.3 uM: | 3.1 ± 1.2 (3) |
| | 3.0 uM: | 21.7 ± 7.9 (3) |
| | 30 uM: | 58.8 ± 16.6 (3) |

TRPML1 —Manual Patch Clamp

Endolysosomal electrophysiology was performed in isolated enlarged endolysosomes using a modified patch-clamp method (Dong et al., 2010). HEK T-REx/GcaMP6f/TRPML1 cells were treated with 0.8 mM PIKfive inhibitor overnight. Whole-endolysosome recordings were performed on manually isolated enlarged endolysosomes (Dong et al., 2010). In brief, a patch pipette was pressed against a cell and quickly pulled away to slice the cell membrane. Enlarged endolysosomes were released into a dish and a gigaseal between the patch pipette and the enlarged endolysosome was obtained. Negative pressure or voltage steps of several hundred millivolts with millisecond duration were then applied to break into the vacuolar membrane.

Bath (internal/cytoplasmic) solution contained 140 mM K-gluconate, 4 mM NaCl, 1 mM EGTA, 2 mM MgCl2, 0.39 mM CaCl₂), 20 mM HEPES (pH was adjusted with KOH to 7.2). The pipette (luminal) solution was standard extracellular solution (modified Tyrode's: 145 mM NaCl, 5 mM KCl, 2 mM CaCl₂), 1 mM MgCl2, 10 mM HEPES, 10 mM MES, 10 mM glucose; the pH was adjusted with NaOH to pH 4.6).

Agonists solubilized 100% DMSO (20 mM) were tested in single concentration (2 μM). Dilution from stock were prepared just before the experiments in the extracellular solution (0.1% final DMSO concentration). DMSO solution was obtained from AppliChem (cat. A3672).

All bath solutions were applied via a perfusion system that allowed us to achieve complete solution exchange within a few seconds.

Data were collected using an HEKA EPC 10 patch-clamp amplifier, and PatchMaster v2x73.5 software (HEKA Elektronik). Whole-endolysosome currents were digitized at 20 kHz. All experiments were conducted at room temperature, and all recordings were analyzed with pClamp 10.7 (Molecular Devices) and GraphPad Prism (V8.00).

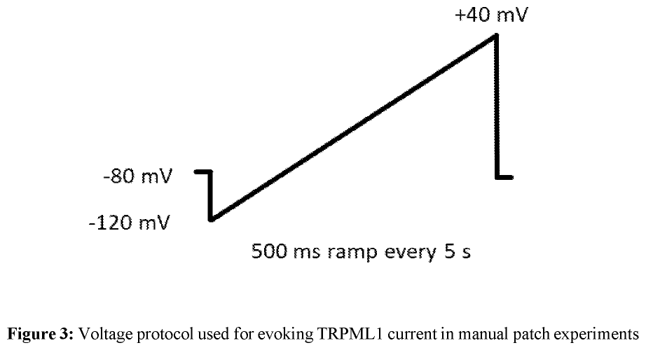

TRPML1 current was evoked using the voltage protocol illustrated in FIG. 3, applied every 5 sec. (10 ms at −80 mV; 500 ms ramp from −120 to +40 mV; 10 ms at −80 mV); in the absence (vehicle period, i.e. 0.1% DMSO) and in the presence of the compound (agonist) under investigation.

TABLE 7
Secondary Assays Agonism-Manual Patch

| | | Lysosomal Patch- |
|---|---|---|
| | | Clamp Current |
| | Concentration | fold increase |
| Example | (uM) | Mean ± SEM (n) |
| 6 | 2.0 | 3.3 ± 0.67 (3) |
| 13 | 30.0 | 27.5 ± 10.8 (2) |
| 57 | 2.0 | 18.0 ± 8.5 (3) |

Autophagy Assay

Buffers and Reagents
Tyrode's buffer with 2 mM Ca²⁺ in-house solution (130 mM NaCl, 5 mM KCl, 2 mM CaCl2), 1 mM MgCl2, 5 mM NaHCO₃, 20 mM HEPES in water at pH 7.4; sterile filtered)
DMSO (Sigma)
Bis-benzimide H 33342 trihydrochloride (Hoechst 33342; Sigma-Aldrich)
Reference molecule (agonist): Torin-1, (#4247, Tocris) stock 1.2 mM in DMSO, stored at −20° C.

Reporter of Autophagy and Cell Line

Reporter Rosella: a fluorescent-based chimera constitutes by two fluorescent proteins, Green and Red variants, acting as a bimodal indicator of pH. The green fluorescent variant of the constructs is pH sensitive (pKa=6.9) while the red variant is not pH sensitive and it is used as reference. The reporter is fused to sequence for autophagosome localization (LC3 tag).

The reporter Rosella have been stably expressed in HeK293 cell line and used as read-out.

Assay Protocol

Experiments have been performed in 384 MTP poly-lysine coated well format. Cells have been seeded in 384-w at a density of 6000 cells/well in 25 μl/well complete growth medium without antibiotics. Twenty-four hours later, cells have been treated with compounds and incubated for further 18 hours. Then the cells have been imaged and assayed for the response to various compounds using the reporter for autophagy expressed in the cells as readout.

The experiments were performed in a 384-well format according to the following procedure:
24 h after seeding, cells were incubated with compounds at the desired concentration, and with the reference molecule (agonist) Torin-1 at a top concentration of 1 μM (max signal), for 18 hours at 37° C. and 5% CO2. The final percentage of DMSO was 0.3% in all the conditions.
Then the culture medium was carefully removed to avoid cells detachment and to discard red phenol into the medium and that can interfere with the measurements.
Staining of the nuclei was obtained by incubating the cells with 8 μM/well of Hoechst 3342 in standard Tyrode's buffer for 20 min at RT
Then the cells were carefully washed two times with standard Tyrode's buffer
Finally, the samples were acquired by recording three fluorescence emission channels (green, red and blue) at 20× magnification and with at least 3-4 fields of view per well in an Operetta CLS microscope (PerkinElmer).
Image analysis was performed by using Harmony software (PerkinElmer). The image analysis involved the following steps: flat-field illumination correction, nuclei segmentation, cell segmentations and identification of vesicles/granules within the cytosol compartments and representing the autophagy vesicles. Measurements of signal intensity ratio (Green to Red) and number of autophagy vesicles per cells were used to obtain information on the effects of compounds on the autophagy flux.
Data from image analysis measurements were finally loaded and analyzed for normalization and fitting procedures in Genedata Screener© software.

TABLE 8
Autophagy assay results
for TRPML1 agonists

| | Secondary |
|---|---|
| | Assays |
| | Autophagy |
| Example | qAC50 − (μM) |
| 6 | 4.4 |
| 13 | 0.52 |
| 19 | 0.60 |
| 20 | 0.46 |
| 33 | 1.3 |
| 40 | 1.3 |
| 46 | 1.3 |
| 57 | 1.8 |
| 58 | 22.4 |
| 65 | 31.4 |
| 68 | 15.3 |
| 75 | 0.17 |
| 76 | 0.059 |
| 77 | 0.25 |
| 96 | 11.2 |
| 99 | 6.0 |

1. A compound of formula (I) 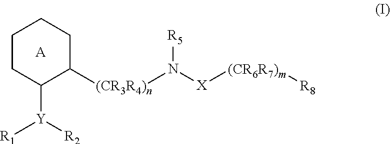
or a stereoisomer thereof, or a salt of any of the foregoing, wherein:
A is a six membered aromatic, heteroaromatic or aliphatic ring, optionally substituted by one or more substituents selected from the group consisting of C₁-C₄ alkyl, C₁-C₄ alkyloxy, C₃-C₆ cycloalkyl, halogen, and cyano.
Y is chosen from CH, or N;
each of R₁ and R₂ independently is a C₁-C₄ alkyl group or R₁ and R₂ taken together with Y form a cyclic compound being a four, five, six or seven membered heteroaliphatic compound, containing at least one nitrogen atom, optionally fused with a six membered aromatic ring, optionally substituted by one or more substituents selected from the group consisting of:
halogen;
C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;
phenyl, benzyl, phenoxy, benzyloxy, pyrazin-2-yl-oxy and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;
a C₁-C₂ alkylene bridge;
R₁₁R₁₂N—CO— wherein each of R₁₁ and R₁₂ is C₁-C₄ alkyl;
X is —CO— or —SO₂—;
each of R₃, R₄, R₆ and R₇ independently is hydrogen or C₁-C₄ alkyl;
R₅ is independently hydrogen or C₁-C₄ alkyl;
each of m and n independently is 0 or 1;
R₈ is a five or six membered monocyclic or a nine or ten membered bicyclic aromatic compound, optionally substituted by one or more substituents selected from the group consisting of halogen, bromo, or pentafluorosulfanyl (SF₅), or trifluoromethylthio (SCF₃), C₁-C₄ alkyl or C₁-C₆cycloalkyl optionally substituted by a cyano group, —NO₂, C₁-C₄ alkyloxy, C₁-C₄ alkylthio, —SO₂NR₉R₁₀ or —NR₉R₁₀, wherein each of R₉ and R₁₀ independently is hydrogen or C₁-C₄ alkyl or R₉ and R₁₀ are taken together to form a five or six membered heteroaliphatic ring, optionally containing one or two oxygen atoms;
provided that:
when X is —CO— then R₅ is C1-C4 alkyl
when X is —SO₂— then R₅ is hydrogen.
when A is phenyl, X is —SO₂, m and n are both 0 and R₁ and R₂ taken together form a piperazine substituted by phenyl optionally substituted as defined above, then R₈ is not phenyl optionally substituted as defined above;
when A is phenyl, X is —SO₂, m and n are both 0 and R₁ and R₂ taken together form an unsubstituted piperidine, pyrrolidine, or azepane, then R₈ is not thiophene optionally substituted as defined above;
when A is cyclohexyl, X is —SO₂, m and n are both 0 and R₁ and R₂ taken together form an unsubstituted morpholine, then R₈ is not phenyl optionally substituted as defined above.

2. A compound of formula (I) according to claim 1 wherein both m and n are 0.

3. A compound of formula (I) or a stereoisomer or a salt thereof according to claim 1 wherein:
A is phenyl or 1,2 disubstituted cyclohexyl;
Y is N and R₁ and R₂ taken together form a cyclic compound selected from piperidine, pyrrolidine, azete, azepane, 2-azabicyclo[2.2.1]heptane, 4-phenylpiperidine, tert-butyl piperazine-1-carboxylate, 4-phenoxypiperidine, 4-(benzyloxy)piperidine, 4-methoxypiperidine, morpholine, dimethylamine, pyrrolidine, 4,4-difluoropiperidine, 3-phenylpiperidine, 3-methoxypiperidine, 1-(4-chloro-2-fluorophenyl)piperazine, 3-phenylpyrrolidine, 2,3-dihydro-1H-indole, 1,2,3,4-tetrahydroquinoline, methyl piperazine-1-carboxylate, 2,2,2-trifluoroethyl piperazine-1-carboxylate, ethyl piperazine-1-carboxylate, 1-(piperazin-1-yl)propan-1-one, 3-methoxy-1-(piperazin-1-yl)propan-1-one, 1-benzoylpiperazine, 2-(piperidin-4-yloxy)pyridine, 2-(piperidin-4-yloxy)pyrazine, tert-butyl piperidine-4-carboxylate, tert-butyl piperidine-1-carboxylate, 4-(pyridin-2-yl)piperidine, 4-(pyrazin-2-yl)piperidine, 4-(thiophene-2-carbonyl)piperazine, 4-(4-methyl-1,3-thiazole-5-carbonyl)piperazine, 4-(piperazin-1-yl)-2-(thiophen-2-yl)ethan-1-one, N-methyl-N-propylpiperazine-1-carboxamide, 4-(thiophene-2-carbonyl)piperazine, 4-(4-methyl-1,3-thiazole-5-carbonyl)piperazine, 4-(piperazin-1-yl)-2-(thiophen-2-yl)ethan-1-one, N-methyl-N-propylpiperazine-1-carboxamide, 3-(piperidin-4-yloxy)pyridine, azepan-1-yl, 4-methoxyazepan-4-yl, 1,4-oxazepan-4-yl, N-propylpiperidine-4-carboxamide, N-(2-methylpropyl)piperidine-4-carboxamide, 2-(piperidin-4-yloxy)pyrimidine, piperidin-4-yl 2,2-dimethylpropanoate.
both m and n are 0;
R₃, R₄, R₆ and R₇ are hydrogen;
X is —CO— or —SO₂—;
R₅ is hydrogen or methyl;
R₈ is selected from phenyl, thiophene or benzothiophene, optionally substituted by dimethylsulfamoyl, methyl, ethyl, propyl, tert-butyl, 1,3-dioxolan-2-yl, cyanomethyl, monoalkylamino, dialkylamino, piperidine-1-sulfonyl, pyrrolidine-1-sulfonyl, morpholine-4-sulfonyl, fluoro, chloro, bromo, SF₅, SCF₃, nitro, and C₁-C₄ alkoxy. provided that:
provided that:
when X is —CO— then R₅ is C1-C4 alkyl
when X is —SO₂— then R₅ is hydrogen.
when A is phenyl, X is —SO₂, m and n are both 0 and R₁ and R₂ taken together form a piperazine substituted by phenyl optionally substituted as defined above, then R₈ is not phenyl optionally substituted as defined above;
when A is phenyl, X is —SO₂, m and n are both 0 and R₁ and R₂ taken together form an unsubstituted piperidine, pyrrolidine, or azepane, then R₈ is not thiophene optionally substituted as defined above;
when A is cyclohexyl, X is —SO₂, m and n are both 0 and R₁ and R₂ taken together form an unsubstituted morpholine, then R₈ is not phenyl optionally substituted as defined above.

4. A compound of formula (I) or a stereoisomer or a salt thereof according to claim 1 wherein:
A is phenyl;
Y is N;
R₁ and R₂ taken together with Y form a cyclic compound selected from piperidine, homopiperidine, pyrrolidine, morpholine, homomorpholine, 2,3-dihydro-1H-indole, 1,2,3,4-tetrahydroisoquinoline optionally substituted by one or more substituents selected from the group consisting of:
halogen;
C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;
phenyl, benzyl, phenoxy, benzyloxy, pyrazin-2-yl-oxy and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;
C₁-C₂ alkylene bridge;
R₁₁R₁₂N—CO— wherein each of R₁₁ and R₁₂ is C₁-C₄ alkyl; or
R₁ and R₂ taken together with Y form a piperazine optionally substituted with —R₁₁R₁₂N—CO— wherein each of R₁₁ and R₁₂ is C₁-C₄ alkyl;
n and m are both 0;
R₃, R₄, R₆ and R₇ are hydrogen;
X is —SO₂— and R₅ is hydrogen; or
X is —CO— and R₅ is methyl;
R₈ is selected from phenyl, thiophene or benzothiophene, thiazole, furan, pyridine, isoxazole, indoline, dihydrobenzofuran, quinolone, optionally substituted by one or more groups selected from dimethylsulfamoyl, methyl, ethyl, propyl, tert-butyl, 1,3-dioxolan-2-yl, mono(C₁-C₆)alkylamino, di (C₁-C₆)alkylamino, methylsulfonyl, and C₁-C₄ alkoxy;
provided that:
when X is —SO₂, and R₁ and R₂ taken together form an unsubstituted piperidine, pyrrolidine, or azepane, then R₈ is not thiophene optionally substituted as defined above.

5. A compound of formula (I) or a stereoisomer or a salt thereof according to claim 1 wherein:
A is cyclohexyl;
Y is N;
R₁ and R₂ taken together with Y form a cyclic compound selected from piperidine, homopiperidine, piperazine, pyrrolidine, morpholine, homomorpholine, 2,3-dihydro-1H-indole, 1,2,3,4-tetrahydroisoquinoline optionally substituted by one or more substituents selected from the group consisting of:
halogen;
C₁-C₄ alkyl, C₁-C₄ alkyloxy, C₁-C₄ alkylcarbonyl, C₁-C₄ alkoxycarbonyl, wherein the C₁-C₄ alkyl group is optionally substituted by one or more halogen or C₁-C₄-alkyloxy;
phenyl, benzyl, phenoxy, benzyloxy, pyrazin-2-yl-oxy and pyridin-2-yl-oxy, thiophene-2-carbonyl, 1,3 thiazole-5-carbonyl, thiophene-2-yl-acetyl, wherein the aromatic ring is optionally substituted by one or more halogen, C₁-C₄ alkyl, or C₁-C₄-alkyloxy;
C₁-C₂ alkylene bridge;
R₁₁R₁₂N—CO— wherein each of R₁₁ and R₁₂ is C₁-C₄ alkyl; or
n and m and n are both 0;
R₃, R₄, R₆ and R₇ are hydrogen;
X is —SO₂— and R₅ is hydrogen; or
X is —CO— and R₅ is methyl;
R₈ is selected from phenyl, thiophene or benzothiophene, thiazole, furan, pyridine, isoxazole, indoline, dihydrobenzofuran, quinolone, substituted by one or more groups selected from dimethylsulfamoyl, methyl, ethyl, propyl, tert-butyl, 1,3-dioxolan-2-yl, mono(C1-C6)alkylamino, di (C1-C6)alkylamino, methylsulfonyl, and C₁-C₄ alkoxy;
when X is —SO₂, and R₁ and R₂ taken together form an unsubstituted morpholine, then
R₈ is not phenyl optionally substituted as defined above.

6. A compound of formula (I) according to claim 1 or a stereoisomer thereof, or a salt of any of the foregoing, selected from the group consisting of:
N-[2-(piperidin-1-yl)phenyl]-1-benzothiophene-2-sulfonamide
4-(dimethylamino)-N-[2-(piperidin-1-yl)phenyl] benzene-1-sulfonamide
N4-[2-(dimethylamino)phenyl]-N1,N1-dimethylbenzene-1,4-disulfonamide
N4-(2-{2-azabicyclo[2.2.1]heptan-2-yl}phenyl)-N1,N1-dimethylbenzene-1,4-disulfonamide
tert-butyl 4-(2-(4-(N,N-dimethylsulfamoyl)-N-methylbenzamido)phenyl)piperazine-1-carboxylate
N1,N1-dimethyl-N4-[2-(piperidin-1-yl)phenyl]benzene-1,4-disulfonamide
2-[4-(dimethylsulfamoyl)phenyl]-N-[2-(pyrrolidin-1-yl)phenyl] acetamide
N4-[2-(2,3-dihydro-1 H-indol-1-yl)phenyl]-N1,N1-dimethylbenzene-1,4-disulfonamide
N1,N1-dimethyl-N4-[2-( 1,2,3,4-tetrahydroisoquinolin-2-yl)phenyl]benzene-1,4-disulfonamide
4-(N,N-dimethylsulfamoyl)-N-methyl-N-(2-(4-(pyridin-2-yloxy)piperidin-1-yl)phenyl)benzamide
3-(l,3-dioxolan-2-yl)-N-[2-(piperidin-1-yl)phenyl]thiophene-2-sulfonamide
N1,N1-dimethyl-N4-[2-(4-phenylpiperidin-1-yl)phenyl] benzene-1,4-disulfonamide
tert-butyl 4-{2-[4-(dimethylsulfamoyl)benzenesulfonamido]phenyl}piperazine-1-carboxylate
1-(2-((4-(N,N-dimethylsulfamoyl)phenyl)sulfonamido)phenyl)-N-methyl-N-propylpiperidine-4-carboxamide
4-(dimethylsulfamoyl)-N-methyl-N-[2-(piperidin-1-yl)phenyl]benzamide
N-(2-{2-azabicyclo[2.2.1]heptan-2-yl}phenyl)-5-tert-butylthiophene-2-sulfonamide
N-(2-{2-azabicyclo[2.2.1]heptan-2-yl}phenyl)-5-ethylthiophene-2-sulfonamide
tert-butyl 4-(2-((4-ethylphenyl)sulfonamido)phenyl)piperazine-1-carboxylate
tert-butyl 1-(2-((4-ethylphenyl)sulfonamido)phenyl)piperidine-4-carboxylate
tert-butyl 4-(2-(4-(N,N-dimethylsulfamoyl)-N-ethylbenzamido)phenyl)piperazine-1-carboxylate
N-(2-{2-azabicyclo[2.2.1]heptan-2-yl}phenyl)-5-ethyl-N-methylthiophene-2-sulfonamide
N1,N1-dimethyl-N4-[2-(piperidin-1-yl)cyclohexyl]benzene-1,4-disulfonamide
3-methyl-N-[2-(piperidin-1-yl)phenyl]benzene-1-sulfonamide
N1,N1-dimethyl-N4-[2-(4-phenoxypiperidin-1-yl)phenyl] benzene-1,4-disulfonamide
N4-{2-[4-(benzyloxy)piperidin-1-yl]phenyl}-N1,N1-dimethylbenzene-1,4-disulfonamide
N4-[2-(4-methoxypiperidin-1-yl)phenyl]-N1,N1-dimethylbenzene-1,4-disulfonamide
N1,N1-dimethyl-N4-[2-(morpholin-4-yl)phenyl]benzene-1,4-disulfonamide
tert-butyl 4-(2-((4-isopropylphenyl)sulfonamido)phenyl)piperazine-1-carboxylate
tert-butyl 1-(2-((4-isopropylphenyl)sulfonamido)phenyl)piperidine-4-carboxylate
N1,N1-dimethyl-N4-(2-(piperidin-1-ylmethyl)phenyl)benzene-1,4-disulfonamide
tert-butyl 1-(2-((4-isopropoxyphenyl)sulfonamido)phenyl)piperidine-4-carboxylate
N1,N1-dimethyl-N4-[2-(pyrrolidin-1-yl)phenyl]benzene-1,4-disulfonamide
N4-[2-(4,4-difluoropiperidin-1-yl)phenyl]-N1,N1-dimethylbenzene-1,4-disulfonamide
N1,N1-dimethyl-N4-[2-(3-phenylpiperidin-1-yl)phenyl]benzene-1,4-disulfonamide
N4-[2-(3-methoxypiperidin-1-yl)phenyl]-N1,N1-dimethylbenzene-1,4-disulfonamide
N1,N1-dimethyl-N4-[2-(3-phenylpyrrolidin-1-yl)phenyl]benzene-1,4-disulfonamide
N1-(2-(4-hydroxypiperidin-1-yl)phenyl)-N4,N4-dimethylbenzene-1,4-disulfonamide
4-isopropoxy-N-(2-(piperidin-1-yl)phenyl)benzenesulfonamide
N1-(2-(4-methoxyazepan-1-yl)phenyl)-N4,N4-dimethylbenzene-1,4-disulfonamide
N1-(2-(4-methoxyazepan-1-yl)phenyl)-N4,N4-dimethylbenzene-1,4-disulfonamide
N1-(2-(4-methoxyazepan-1-yl)phenyl)-N4,N4-dimethylbenzene-1,4-disulfonamide
N1-(2-(l,4-oxazepan-4-yl)phenyl)-N4,N4-dimethylbenzene-1,4-disulfonamide
N1-(2-(4-fluoropiperidin-1-yl)phenyl)-N4,N4-dimethylbenzene-1,4-disulfonamide
N1,N1-dimethyl-N4-(2-(4-propoxypiperidin-1-yl)phenyl)benzene-1,4-disulfonamide
tert-butyl 4-(2-(4-(N,N-dimethylsulfamoyl)-N-isopropylbenzamido)phenyl)piperazine-1-carboxylate
tert-butyl 4-(2-((2-methylphenyl)sulfonamido)phenyl)piperazine-1-carboxylate
N-(2-{2-azabicyclo[2.2.1]heptan-2-yl}phenyl)-4-(dimethylsulfamoyl)-N-methylbenzamide
tert-butyl 4-(2-((2,4-dimethylphenyl)sulfonamido)phenyl)piperazine-1-carboxylate
tert-butyl 4-(2-((3,4-dimethylphenyl)sulfonamido)phenyl)piperazine-1-carboxylate
methyl4-{2-[4-(dimethylsulfamoyl)benzenesulfonamido]phenyl}piperazine-1-carboxylate
2,2,2-trifluoroethyl 4-{2-[4-(dimethylsulfamoyl)benzenesulfonamido]phenyl}piperazine-1-carboxylate
tert-butyl 4-(2-((3,5-dimethylphenyl)sulfonamido)phenyl)piperazine-1-carboxylate
N1,N1-dimethyl-N4-[2-(4-phenylpiperidin-1-yl)cyclohexyl]benzene-1,4-disulfonamide
tert-butyl 4-(2-((2,4-dimethylthiazole)-5-sulfonamido)phenyl)piperazine-1-carboxylate
N1,N1-dimethyl-N4-[2-(4-phenoxypiperidin-1-yl)cyclohexyl]benzene-1,4-disulfonamide
tert-butyl 4-(2-((2,5-dimethylfuran)-3-sulfonamido)phenyl)piperazine-1-carboxylate
tert-butyl4-(2-((2,5-dimethylthiophene)-3-sulfonamido)phenyl)piperazine-1-carboxylate
tert-butyl 4-(2-((5-methylpyridine)-3-sulfonamido)phenyl)piperazine-1-carboxylate
N1,N1-dimethyl-N4-[2-(morpholin-4-yl)cyclohexyl]benzene-1,4-disulfonamide
N1,N1-dimethyl-N4-[2-(4-phenoxypiperidin-1-yl)cyclohexyl]benzene-1,4-disulfonamide
N1,N1-dimethyl-N4-[2-(4-phenoxypiperidin-1-yl)cyclohexyl]benzene-1,4-disulfonamide
tert-butyl 4-(2-((3,5-dimethylisoxazole)-4-sulfonamido)phenyl)piperazine-1-carboxylate
2,4-dimethyl-N-(2-(4-(pyridin-2-yloxy)piperidin-1-yl)phenyl)benzenesulfonamide
2,4-dimethyl-N-(2-(4-propoxypiperidin-1-yl)phenyl)benzenesulfonamide
2,5-dimethyl-N-(2-(4-propoxypiperidin-1-yl)phenyl)furan-3-sulfonamide
2,5-dimethyl-N-(2-(4-propoxypiperidin-1-yl)phenyl)thiophene-3-sulfonamide
ethyl 4-{2-[4-(dimethylsulfamoyl)benzenesulfonamido]phenyl}piperazine-1-carboxylate
N1,N1-dimethyl-N4-[2-(4-propanoylpiperazin-1-yl)phenyl]benzene-1,4-disulfonamide
tert-butyl 4-(2-((l-methylindoline)-5-sulfonamido)phenyl)piperazine-1-carboxylate
N4-[2-(4-benzoylpiperazin-1-yl)phenyl]-N1,N1-dimethylbenzene-1,4-disulfonamide
N1,N1-dimethyl-N4-{[2-(piperidin-1-yl)phenyl]methyl}benzene-1,4-disulfonamide
N1,N1-dimethyl-N4-{2-[4-(pyridin-2-yloxy)piperidin-1-yl]phenyl}benzene-1,4-disulfonamide
t-butyl 1-{2-[4-(dimethylsulfamoyl)benzenesulfonamido]phenyl}piperidine-4-carboxylate
N1,N1-dimethyl-N4-{2-[4-(pyrazin-2-yloxy)piperidin-1-yl]phenyl}benzene-1,4-disulfonamide
t-butyl 4-{2-[4-(dimethylsulfamoyl)benzenesulfonamido]phenyl}piperidine-1-carboxylate
N1,N1-dimethyl-N4-{2-[4-(thiophene-2-carbonyl)piperazin-1-yl] phenyl}benzene-1,4-disulfonamide
N1,N1-dimethyl-N4-{2-[4-(4-methyl-l,3-thiazole-5-carbonyl)piperazin-1-yl]phenyl}benzene-1,4-disulfonamide
N1,N1-dimethyl-N4-(2-{4-[2-(thiophen-2-yl)acetyl]piperazin-1-yl}phenyl)benzene-1,4-disulfonamide
4-{2-[4-(dimethylsulfamoyl)benzenesulfonamido]phenyl}-N-methyl-N-propylpiperazine-1-carboxamide
t-butyl 4-[2-(4-methylbenzenesulfonamido)phenyl]piperazine-1-carboxylate
t-butyl 4-[2-(4-methoxybenzenesulfonamido)phenyl]piperazine-1-carboxylate
N4-[2-(azepan-1-yl)phenyl]-N1,N1-dimethylbenzene-1,4-disulfonamide
N1,N1-dimethyl-N4-{2-[4-(pyridin-3-yloxy)piperidin-1-yl]phenyl}benzene-1,4-disulfonamide
1-{2-[4-(dimethylsulfamoyl)benzenesulfonamido]phenyl}-N-propylpiperidine-4-carboxamide
1-{2-[4-(dimethylsulfamoyl)benzenesulfonamido]phenyl}-N-(2-methylpropyl)piperidine-4-carboxamide
N1,N1-dimethyl-N4-{2-[4-(pyrimidin-2-yloxy)piperidin-1-yl]phenyl}benzene-1,4-disulfonamide
1-[2-(4-methylbenzenesulfonamido)phenyl]piperidin-4-yl 2,2-dimethylpropanoate
1-[2-(4-methoxybenzenesulfonamido)phenyl]piperidin-4-yl 2,2-dimethylpropanoate
tert-butyl 4-(2-((2-methyl-2,3-dihydrobenzofuran)-5-sulfonamido)phenyl)piperazine-1-carboxylate
tert-butyl 4-(2-((2-methyl-2,3-dihydrobenzofuran)-5-sulfonamido)phenyl)piperazine-1-carboxylate
tert-butyl 4-(2-((2-methyl-2,3-dihydrobenzofuran)-5-sulfonamido)phenyl)piperazine-1-carboxylate
tert-butyl 4-(2-((8-methylquinoline)-5-sulfonamido)phenyl)piperazine-1-carboxylate
tert-butyl 4-(2-(N-isopropyl-4-(methylsulfonyl)benzamido)phenyl)piperazine-1-carboxylate
2,5-dimethyl-N-(2-(4-(pyridin-2-yloxy)piperidin-1-yl)phenyl)thiophene-3-sulfonamide
6-methyl-N-(2-(4-(pyridin-2-yloxy)piperidin-1-yl)phenyl)pyridine-3-sulfonamide
2,5-dimethyl-N-(2-(4-(pyridin-2-yloxy)piperidin-1-yl)phenyl)thiazole-4-sulfonamide
N1,N1-dimethyl-N4-(2-(2-phenylmorpholino)phenyl)benzene-1,4-disulfonamide
N1-(2-(2-benzylmorpholino)phenyl)-N4,N4-dimethylbenzene-1,4-disulfonamide
N-isopropyl-4-(methylsulfonyl)-N-(2-(4-(pyrazin-2-yloxy)piperidin-1-yl)phenyl)benzamide
methyl 1-(2-((4-(N,N-dimethylsulfamoyl)phenyl)sulfonamido)phenyl)piperidine-4-carboxylate
N1,N1-dimethyl-N4-(2-(4-(pyridin-4-yloxy)piperidin-1-yl)phenyl)benzene-1,4-disulfonamide

7. A process for preparing a compounds of formula (I) according to claim 1 or a stereoisomer thereof, or a salt of any of the foregoing comprising the step of reacting a compound of formula (II) or formula (III): 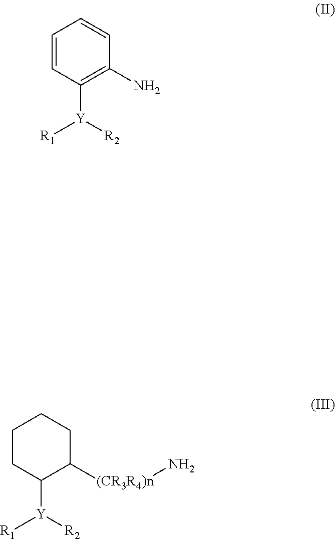
wherein Y, R₁, R₂, R₃, R₄ and n are as defined in claim 1, with a compound of formula (IV) or formula (V) 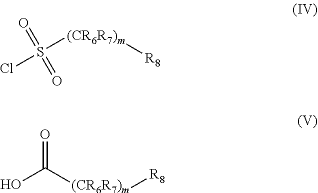
wherein R₆, R₇, m and R₈ are as defined in claim 1.

8. A process for preparing a compounds of formula (I) according to claim 1 or a stereoisomer thereof, or a salt of any of the foregoing comprising the step of reacting a compound of formula (VI): 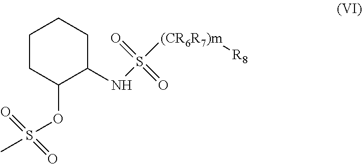
wherein R₆, R₇, m and R₈ are as defined above; with a suitably substituted amine.

9. A process for preparing a compounds of formula (I) according to claim 1 or a stereoisomer thereof, or a salt of any of the foregoing comprising the step of reacting a compound of formula (VII) or compound of formula (VIII) with CH₃I or CH₃CH₂I or (CH₃)₂CHI 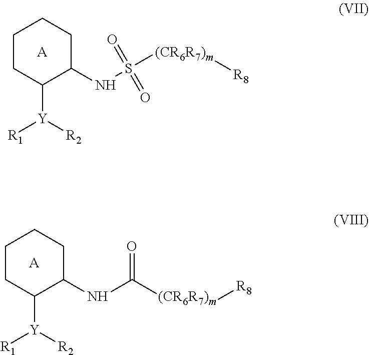
wherein A, Y, R₁, R₂, R₆, R₇, m and R₈ are as defined in claim 1.

10. A process for preparing a compounds of formula (I) according to claim 1 or a stereoisomer thereof, or a salt of any of the foregoing comprising the step of reacting a compound of formula (IX) 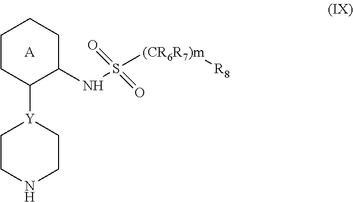
wherein A, Y, R₆, R₇, m and R₈ are a fined above, with a compound of formula (X) 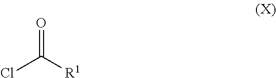
wherein R₁₁ and R₁₂ is as defined above.

11. A pharmaceutical composition comprising a compound of formula (I) according to claim 1, or a stereoisomer thereof, or a salt of any of the foregoing and a pharmaceutically acceptable carrier.

12. A medicament comprising the compound of formula (I) according to claim 1, or a stereoisomer thereof, or a salt of any of the foregoing.

13. A method for treatment of a TRPML1-mediated disorder or disease, comprising administering an effective amount of the compound of formula (I) according to claim 1, or a stereoisomer thereof, or a salt of any of the foregoing, to a patient in need thereof.

14. The method of claim 13, wherein the TRPML1-mediated disorder or disease is selected from diseases of aging, bone diseases, cardiovascular diseases, congenital developmental disorders, eye diseases, hematological and solid malignancies, infectious diseases, inflammatory diseases, liver diseases, metabolic diseases, neurological or neurodegenerative diseases, pancreatitis, renal diseases, skeletal muscle disorders, obesity, lysosomal storage diseases and pulmonary Diseases

15. The method of claim 13, wherein the TRPML1-mediated disorder or disease is selected from Aicardi-Goutiéres syndrome, Alzheimer's Disease, amyotrophic lateral sclerosis, ataxia-telangiectasia, autism spectrum disorders, Batten Disease, bipolar disorder, cerebral ataxia, Charcot-Marie-Tooth variant diseases, Chronic Wasting Disease, corticobasal degeneration, corticobasal syndrome, bovine spongiform encephalopathy, Creutzfeldt-Jacob Disease, Danon Disease, Duchenne Muscular Dystrophy, Exotic ungulate encephalopathy, Fabre Disease, Fatal Famial insomnia, Fridreich Ataxia, Feline spongiform Encephalopathy, Fragile X, Frontal temporal dementia, Gaucher Disease, Gerstmann-Straussler-Scheinker Disease, Giant axonal neuropathy, GM1 and GM2 gangliosidosis, Huntington's Disease, Infantile Refsum Disease, JUNQ and IPOD, Kuru, Leukoencephalopathy, Lewy Body Dementia, locomotor ataxia, Lyme disease, Machado Joseph Disease, major depressive disorder, MPS-III, Mucolipidosis, multiple sulfatase deficiency, multiple systems atrophy, myofibrillar myopathies, myotonic dystrophy, Niemann-Pick Disease, Parkinson's Disease, Parkinsonism, Pick's disease, polyglutamine diseases, Pompe Disease, pontocerebellar hypoplasia, prion diseases, progressive nuclear palsy, pyruvate dehydrogenase deficiency, Sandhoff Disease, Schizophrenia, Scrapie, Shy-Drager syndrome, spinal muscular atrophy, spinocerebellar ataxias, sporadic familial insomnia, subacute degeneration of the spinal cord, subacute sclerosing panencephalitis, Tay-Sachs disease, transneuronal degeneration, and vascular dementia.
